(19) United States
(12) Patent Application Publication
(10) Pub. No.: US 2021/0332143 A1
(43) Pub. Date: Oct. 28, 2021

(54) COMBINATION THERAPY FOR CANCER COMPRISING PD-1 AXIS BINDING ANTAGONIST AND IL6 ANTAGONIST

(71) Applicant: Genentech, Inc., South San Francisco, CA (US)

(72) Inventors: Mahrukh Huseni, South San Francisco, CA (US); Joanna E. Klementowicz, South San Francisco, CA (US); Yijin Li, South San Francisco, CA (US); Li-Fen Liu, South San Francisco, CA (US); Sanjeev Mariathasan, South San Francisco, CA (US); Mark Merchant, South San Francisco, CA (US); Luciana Molinero, South San Francisco, CA (US); Lifen Wang, South San Francisco, CA (US); Nathaniel West, Pacifica, CA (US); Patrick Williams, San Francisco, CA (US); Chi Yung Yuen, South San Francisco, CA (US); Edward Namserk Cha, South San Francisco, CA (US); Yulei Wang, South San Francisco, CA (US)

(73) Assignee: Genentech, Inc., South San Francisco, CA (US)

(21) Appl. No.: 17/249,530

(22) Filed: Mar. 4, 2021

Related U.S. Application Data
(60) Provisional application No. 62/986,050, filed on Mar. 6, 2020.
(60) Provisional application No. 63/059,054, filed on Jul. 30, 2020.
(60) Provisional application No. 63/081,583, filed on Sep. 22, 2020.

(51) Int. Cl. C07K 16/28; A61P 35/00; A61K 31/337; A61K 47/64

(52) U.S. Cl. CPC C07K 16/2866; C07K 16/2827; A61K 2039/575; A61K 31/337; A61K 47/643; A61P 35/00

(57) ABSTRACT

This application discloses methods and compositions for use in treating cancer, including breast cancer (such as metastatic triple negative breast cancer, mTNBC), urothelial carcinoma, renal cell carcinoma, and liver cancer (hepatocellular carcinoma, HCC) with the combination of a PD-1 axis binding antagonist (e.g., a PD-L1 binding antibody such as atezolizumab) and an IL6 antagonist (e.g. an anti-IL6 receptor antibody such as tocilizumab), optionally further comprising a VEGF antagonist (e.g. an anti-VEGF antibody such as bevacizumab). Optionally, the patient has C-reactive protein (CRP) and/or IL-6 level(s) above the upper limit of normal. Optionally, the cancer is PD-L1 positive.

### CROSS REFERENCE TO RELATED APPLICATIONS

This application claims the benefit U.S. Provisional Application No. 62/986,050, filed Mar. 6, 2020; U.S. Provisional Application No. 63/059,054, filed Jul. 30, 2020; and U.S. Provisional Application No. 63/081,583 filed on Sep. 20, 2020, which are incorporated by reference in entirety.

### SEQUENCE LISTING

The instant application contains a sequence listing submitted via efs-web and is hereby incorporated by reference in its entirety. Said ASCII copy, created Feb. 25, 2021, is named P35974US3SEQLIST.txt, and is 31,818 bytes in size.

### FIELD OF THE INVENTION

This invention relates to methods of treating cancer including breast cancer (such as metastatic triple negative breast cancer, mTNBC), urothelial carcinoma (UC), renal cell carcinoma (RCC), and liver cancer (e.g. hepatocellular carcinoma, HCC). The invention also relates to combination therapy for cancer comprising a PD-1 axis binding antagonist (e.g., atezolizumab) and an IL6 antagonist (e.g. tocilizumab), optionally with further chemotherapy or with vascular endothelial growth factor (VEGF) antagonist (e.g. an anti-VEGF antibody, such as bevacizumab). The invention further relates to treating certain cancer patient subpopulations with the combination, including patients with high CRP and/or IL-6 level(s), optionally also having a PD-L1 positive tumor. The invention also relates to methods of reducing or preventing therapeutic resistance to a PD-1 axis binding antagonist (e.g. an anti-PD-L1 antibody such as atezolizumab) in a cancer patient comprising administering the PD-1 axis binding antagonist in combination with an IL-6 antagonist (such as an anti-IL6 receptor antibody like tocilizumab), optionally where the patient has abnormal CRP and/or IL-6 level(s). Optionally, the patient's cancer is PD-L1 positive.

### BACKGROUND OF THE INVENTION

Cancer remains one of the deadliest threats to human health. Cancers, or malignant tumors, metastasize and grow rapidly in an uncontrolled manner, making timely detection and treatment extremely difficult.

Worldwide, urothelial carcinoma (UC) is the most common cancer of the urinary system. The majority of urothelial tumors arise in the bladder with the remainder originating in the renal pelvis, urethra, or ureter. Transitional cell carcinoma (TCC) is the most common histologic subtype associated with bladder cancer and accounts for >90% of all UC cases in the industrialized world. Globally, there were an estimated 429,793 new cases of bladder cancer and 165,084 deaths in 2012. In 2017, it was estimated that there would be 79,030 new cases and 16,870 deaths from bladder cancer in the United States. Poor prognostic factors for survival in patients with metastatic urothelial carcinoma (mUC) include advanced stage of disease at the time of initial diagnosis, Karnofsky Performance Status (KPS) <80%, and visceral metastasis (i.e., lung, liver, or bone). The presence of these unfavorable features was associated with a median survival of 4 months compared with 18 months in patients without these features. The overall 5-year survival rate for mUC is approximately 5.2%. Historically, platinum-based chemotherapy has been the standard of care for patients with previously untreated mUC. In an effort to develop a less toxic regimen, the combination of gemcitabine and cisplatin (GC) was subsequently developed. Despite the observed efficacy of cisplatin-based combination chemotherapy, up to 50% of patients are ineligible to receive cisplatin because of baseline comorbidities and impaired functional status.

Breast cancer is the most frequent cancer diagnosed in women. Breast cancer accounts for approximately 15% (approximately 626,700 cases) of all cancer deaths and is the most common cause of cancer-related mortality in women, with a 5-year survival rate of approximately 15% following metastatic diagnosis. The treatment algorithm for patients with metastatic breast cancer is based on several factors that include clinical, pathologic, and histologic characteristics such as the presence or absence of HER2 amplification, hormone receptor status, PD-L1 status, prior response to and/or failure of hormonal agents, number and specific sites of metastatic disease, and treatment history in both the metastatic and adjuvant settings. Several cytotoxic chemotherapy agents have shown activity in metastatic breast cancer, including anthracyclines, taxanes, carboplatin, gemcitabine, capecitabine, vinorelbine, eribulin, and ixabepilone. The response rates and progression-free intervals observed with these agents vary depending on the extent and type of prior therapy and the extent of metastatic disease, as well as the biology of the disease. In general, anthracycline-based combination therapy and taxanes, such as paclitaxel and docetaxel, are believed to show the greatest activity. Given the use of regimens containing anthracyclines in the adjuvant setting and the risk of cardiotoxicity associated with repeated courses, taxanes are now the most commonly used chemotherapy agent for patients with locally advanced or metastatic disease, particularly in the front-line setting. Triple-negative breast cancer (TNBC) may be simply defined by the absence of immunostaining for estrogen receptor (ER), progesterone receptor (PR), and HER2. Overall, approximately 15%-20% of breast cancers are classified as TNBC. Large-scale comprehensive genomic analyses have characterized the heterogeneous nature of TNBCs and their diverse gene-expression patterns and underlying genomic changes, but these insights have not yet provided clear guidance for the identification of clinically effective targeted therapies. Unfortunately, TNBCs are more likely to have aggressive features, such as a high proliferative rate, and exhibit an invasive phenotype.

Programmed Death-Ligand 1 (PD-L1) The PD-L1 pathway serves as an immune checkpoint to temporarily dampen immune responses in states of chronic antigen stimulation, such as chronic infection or cancer. PD-L1 is an extracellular protein that downregulates immune responses through binding to its two receptors, PD-1 and B7-1. PD-1 is an inhibitory receptor expressed on T cells following T-cell activation, and expression is sustained in states of chronic stimulation (Blank et al. Interaction of PD-L1 on tumor cells with PD-1 on tumor-specific T cells as a mechanism of immune evasion: implications for tumor immunotherapy. Cancer Immunol Immunother 54:307-14 (2005); Keir et al. PD-1 and its ligands in tolerance and immunity. Annu Rev Immunol 2008; 26:677-704 (2008). B7-1 is a molecule expressed on antigen-presenting cells and activated T cells. Binding of PD-L1 to PD-1 and B7-1 inhibits T-cell proliferation and activation, cytokine production, and cytolytic activity, leading to the functional inactivation or exhaustion of T cells (Butte et al. Programmed death-1 ligand 1 interacts specifically with the B7-1 costimulatory molecule to inhibit T cell responses. Immunity 27:111-22 (2007); Yang et al. The novel costimulatory programmed death ligand 1/B7.1 pathway is functional in inhibiting alloimmune responses in vivo. J Immunol 187:1113-9 (2011)).

Overexpression of PD-L1 on tumor cells has been reported to impede anti-tumor immunity, resulting in immune evasion (Blank and Mackensen. Contribution of the PD-L1/PD-1 pathway to T cell exhaustion: an update on implications for chronic infections and tumor evasion. Cancer Immunol Immunother 56:739-45 (2007)).

Therefore, interruption of the PD-L1 pathway represents an attractive strategy for restoring tumor-specific T-cell immunity.

Atezolizumab is programmed death-ligand 1 (PD-L1) blocking antibody. More specifically, it is an Fc-engineered, humanized, non-glycosylated IgG1 kappa immunoglobulin that has a calculated molecular mass of 145 kDa. Atezolizumab is approved for the following uses in the United States:
1. Urothelial Carcinoma: for the treatment of adult patients with locally advanced or metastatic urothelial carcinoma who:
   a. are not eligible for cisplatin-containing chemotherapy and whose tumors express PD-L1 (PD-L1 stained tumor-infiltrating immune cells [IC] covering 5% of the tumor area), as determined by an FDA-approved test, or
   b. are not eligible for any platinum-containing chemotherapy regardless of PD-L1 status, or o have disease progression during or following any platinum-containing chemotherapy, or within 12 months of neoadjuvant or adjuvant chemotherapy.
2. Non-Small Cell Lung Cancer (NSCLC):
   a. In combination with bevacizumab, paclitaxel, and carboplatin, for the firstline treatment of adult patients with metastatic non-squamous NSCLC with no EGFR or ALK genomic tumor aberrations.
   b. In combination with paclitaxel protein-bound and carboplatin for the firstline treatment of adult patients with metastatic non-squamous NSCLC with no EGFR or ALK genomic tumor aberrations
   c. For the treatment of adult patients with metastatic NSCLC who have disease progression during or following platinum-containing chemotherapy. Patients with EGFR or ALK genomic tumor aberrations should have disease progression on FDA-approved therapy for NSCLC harboring these aberrations prior to receiving atezolizumab.
3. Triple-Negative Breast Cancer (TNBC): in combination with paclitaxel protein-bound for the treatment of adult patients with unresectable locally advanced or metastatic TNBC whose tumors express PD-L1 (PD-L1 stained tumor-infiltrating immune cells [IC] of any intensity covering 1% of the tumor area), as determined by an FDA approved test.
4. Small Cell Lung Cancer (SCLC): in combination with carboplatin and etoposide, for the first-line treatment of adult patients with extensive-stage small cell lung cancer (ES-SCLC).

Atezolizumab can be administered by various dosing schedules including: 840 mg every 2 weeks, 1200 mg every 3 weeks, and 1680 mg every 4 weeks, as a single agent or with chemotherapy and/or bevacizumab.

The following clinical trials involve Atezolizumab:

| Study | Clintrial.gov | Cancer type |
|---|---|---|
| MORPHEUS NSCLC | NCT03337698 | Metastatic NSLCL |
| MORPHEUS mUC | NCT03869190 | locally advanced or metastatic urothelial bladder cancer |
| | | (mUC), after failure with platinum-containing |
| | | chemotherapy |
| PCD4989g | NCT01375842 | locally advanced or metastatic solid malignancies, |
| | | including triple negative breast cancer (mTNBC) |
| IMvigor210 | NCT02108652 | mUC |
| IMvigor211 | NCT02302807 | mUC, with prior platinum-based chemotherapy |
| IMmotion150 | NCT01984242 | locally advanced or metastatic renal cell |
| | | carcinoma (mRCC), with or without bevacizumb |
| IMbrave150 | NCT03434379 | Untreated locally advanced metastatic hepatocellular |
| | | carcinoma (HOC), with bevacizumab |
| MORPHEUS Liver | NCT04524871 | Advanced liver cancers |
| MORPHEUS PDAC | NCT03193190 | Metastatic pancreatic ductal adenocarcinoma (PDAC) |

### Interleukin 6 (IL-6) Receptor

Interleukin-6 (IL-6) is a proinflammatory, multifunctional cytokine produced by a variety of cell types. IL-6 is involved in such diverse processes as T-cell activation, B-cell differentiation, induction of acute phase proteins, stimulation of hematopoietic precursor cell growth and differentiation, promotion of osteoclast differentiation from precursor cells, proliferation of hepatic, dermal and neural cells, bone metabolism, and lipid metabolism (Hirano T. Chem Immunol. 51:153-180 (1992); Keller et al. Frontiers Biosci. 1: 340-357 (1996); Metzger et al. Am J Physiol Endocrinol Metab. 281: E597-E965 (2001); Tamura et al. Proc Natl Acad Sci USA. 90:11924-11928 (1993); Taub R. J Clin Invest 112: 978-980 (2003)). IL-6 has been implicated in the pathogenesis of a variety of diseases including autoimmune diseases, osteoporosis, neoplasia, and aging (Hirano, T. (1992), supra; and Keller et al., supra). IL-6 exerts its effects through a ligand-specific receptor (IL-6R) present both in soluble and membrane-expressed forms.

Elevated IL-6 levels have been reported in the serum and synovial fluid of RA patients, indicative of production of IL-6 by the synovium (Irano et al. Eur J Immunol. 18:1797-1801 (1988); and Houssiau et al. Arthritis Rheum. 1988; 31:784-788 (1988)). IL-6 levels correlate with disease activity in RA (Hirano et al. (1988), supra), and clinical efficacy is accompanied by a reduction in serum IL-6 levels (Madhok et al. Arthritis Rheum. 33:S154. Abstract (1990)).

Tocilizumab (TCZ) is a recombinant humanized monoclonal antibody of the immunoglobulin IgG1 subclass which binds to human IL-6R. Tocilizumab is approved for:
1. Rheumatoid Arthritis (RA): Adult patients with moderately to severely active rheumatoid arthritis who have had an inadequate response to one or more Disease-Modifying Anti-Rheumatic Drugs (DMARDs).
2. Giant Cell Arteritis (GCA): Adult patients with giant cell arteritis.
3. Polyarticular Juvenile Idiopathic Arthritis (pJIA): Patients 2 years of age and older with active polyarticular juvenile idiopathic arthritis.
4. Systemic Juvenile Idiopathic Arthritis (sJIA): Patients 2 years of age and older with active systemic juvenile idiopathic arthritis.
5. Cytokine Release Syndrome (CRS): Adults and pediatric patients 2 years of age and older with chimeric antigen receptor (CAR) T cell-induced severe or life-threatening cytokine release syndrome.

Tocilizumab was combined with carboplatin and doxorubicin and interferon-α2b in patients with recurrent epithelial ovarian cancer (Dijkgraaf et al. A phase 1 trial combining carboplatin/doxorubicin with tocilizumab, an anti-IL-6R monoclonal antibody, and interferon-α2β in patients with recurrent epithelial ovarian cancer. Ann Oncol 26:2141-9 (2015)). In this dose-escalation study, patients received carboplatin and doxorubicin for 6 cycles. For the first 3 cycles, patients received 1, 2, 4, or 8 mg/kg of IV tocilizumab every 4 weeks. At the 8-mg/kg dose of tocilizumab, interferon-α2b was added after 3 patients had received and tolerated the 8-mg/kg dose without interferon-α2b. Twenty-one patients were assessed for response and of these patients, 3 patients had a complete response, 8 had a partial response, 6 had stable disease, and 3 had progressive disease.

### Vascular Endothelial Growth Factor (VEGF)

VEGF is an angiogenic factor. Bevacizumab is an anti-vascular endothelial growth factor (VEGF) antibody indicated for the treatment of:
Metastatic colorectal cancer: in combination with intravenous fluorouracil based chemotherapy for first- or second-line treatment.
Metastatic colorectal cancer: in combination with fluoropyrimidineirinotecan- or fluoropyrimidine-oxaliplatin-based chemotherapy for second-line treatment in patients who have progressed on a first-line bevacizumab-containing regimen.
Unresectable, locally advanced, recurrent or metastatic non-squamous non-small cell lung cancer: in combination with carboplatin and paclitaxel for first-line treatment.
Recurrent glioblastoma: in adults.
Metastatic renal cell carcinoma: in combination with interferon alfa.
Persistent, recurrent, or metastatic cervical cancer: in combination with paclitaxel and cisplatin, or paclitaxel and topotecan.
Epithelial ovarian, fallopian tube, or primary peritoneal cancer: in combination with carboplatin and paclitaxel, followed by bevacizumab as a single agent, for stage III or IV disease following initial surgical resection or in combination with paclitaxel, pegylated liposomal doxorubicin, or topotecan for platinum-resistant recurrent disease who received no more than 2 prior chemotherapy regimens or in combination with carboplatin and paclitaxel or carboplatin and gemcitabine, followed by bevacizumab as a single agent, for platinumsensitive recurrent disease.
Hepatocellular Carcinoma (HCC): in combination with atezolizumab for the treatment of patients with unresectable or metastatic HCC who have not received prior systemic therapy.

Approved dosing regimens for bevacizumab include 5 mg/kg every 2 weeks, 7.5 mg/kg every 2 weeks, 10 mg/kg every 2 weeks, and 15 mg/kg every 3 weeks.

### OTHER PUBLICATIONS

Elevated plasma IL-6 correlated with reduced sensitivity to PD-1 blockade in small cohorts of melanoma patients treated with the anti-PD-1 antibody nivolumab (Tsukamoto et al. Cancer Res 78: 5011-5022 (2018); and Weber et al. Journal of Clinical Oncology 37: 100-100 (2019)). However, the clinical relevance of IL-6 to anti-PD-1 treatment was not evaluated in other indications (such as breast cancer, urothelial carcinoma, or renal cell carcinoma) or in large randomized trials, the relevance to an anti-PD-L1 antibody such as atezolizumab was not investigated, nor was the effect of an anti-IL6 receptor antibody (such as tocilizumab) versus an anti-IL6 antibody (MP5-20F3) evaluated.

Anti-IL-6 and anti-PD-L1 was evaluated in murine models of pancreatic cancer (Mace et al. IL-6 and PD-L1 antibody blockade combination therapy reduces tumor progression in murine model of pancreatic cancer. Gut 2016; epub ahead of print: doi: 10.1136/gutjnl-2016-311585).

### SUMMARY OF THE INVENTION

In a first aspect, the invention concerns a method of treating a cancer patient comprising administering to the patient a combination of an IL-6 antagonist and a PD-1 axis binding antagonist in an amount effective to treat the cancer.

Examples of cancers to be treated with the combination include, without limitation, breast cancer, such as triple negative breast cancer (TNBC), bladder cancer, urothelial carcinoma, kidney cancer, renal cell carcinoma, and hepatocellular carcinoma.

Other examples of cancer include: a liver cancer, a lung cancer, a colorectal cancer, an ovarian cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In one embodiment, the cancer is not melanoma or not pancreatic cancer.

In one embodiment, the patient has C-reactive protein (CRP) level above the upper limit of normal. For example, the patient may have 3 mg/L CRP, e.g. mg/L CRP. Various assays for measuring CRP are available. In one embodiment, the CRP is measured by enzyme-linked immunosorbent assay (ELISA) assay and the sample is a blood sample from the patient.

In one embodiment, the patient has IL-6 level above the upper limit of normal. For example, the patient may have 0 pg/mL IL-6, e.g. pg/mL IL-6. Various assays are available for measuring IL-6. In one embodiment, IL-6 is measured by enzyme-linked immunosorbent assay (ELISA) assay and the sample is a blood sample from the patient.

In one embodiment, the patient expresses PD-L1. For example, the patient may have PD-L1 stained tumor cells (TC) and/or tumor-infiltrating immune cells (IC), e.g. where the PD-L1 stained IC cover 1% of the tumor area, e.g. 5% of the tumor area.

In one embodiment, the patient has CRP and/or IL-6 above the upper limit of normal and expresses PD-L1.

In one embodiment, the IL-6 antagonist is an anti-IL6 receptor antibody, e.g. tocilizumab, satralizumab, sarilumab, NI-1201, or vobarilizumab, preferably tocilizumab.

In one embodiment, the PD-L1 axis binding antagonist is a PD-L1 binding antagonist, e.g. which inhibits the binding of PD-L1 to both PD-1 and B7-1 and/or is an antibody. Examples of PD-L1 binding antibodies contemplated herein include atezolizumab, MDX-1105, MEDI4736 (durvalumab), or MSB0010718C (avelumab), atezolizumab being preferred.

In one embodiment, the treatment results in an increased abundance of CD8⁺ T cells in the patient relative to that of a subject who has not been administered the IL-6 antagonist.

In one embodiment, the treatment reduces or prevents therapeutic resistance to the PD-1 axis binding antagonist.

In another embodiment, the invention concerns a method of treating a cancer patient comprising administering to the patient a combination of an anti-IL6 receptor antibody and an anti-PD-L1 antibody in an amount effective to treat the cancer. For example, the cancer can be breast cancer, urothelial carcinoma, or renal cell carcinoma.

In yet another embodiment, the invention provides a method of treating a cancer patient with C-reactive protein (CRP) level above the upper limit of normal comprising administering to the patient a combination of an anti-IL6 receptor antibody and an anti-PD-L1 antibody in an amount effective to treat the cancer.

In another embodiment, the invention concerns a method of treating advanced urothelial carcinoma in a cancer patient comprising administering to the patient a combination of tocilizumab and atezolizumab in an amount effective to treat the cancer.

In another embodiment, the invention concerns a method of treating triple negative breast cancer (TNBC) in a cancer patient comprising administering to the patient a combination of tocilizumab, atezolizumab, and chemotherapy (e.g. a taxane such as nanoparticle albumin-bound paclitaxel (nab paclitaxel)) in an amount effective to treat the cancer.

In yet another embodiment, the invention provides a method of reducing or preventing therapeutic resistance to a PD-1 axis binding antagonist (e.g. an anti-PD-L1 antibody, e.g. atezolizumab) in a cancer patient (e.g. a breast cancer patient, urothelial carcinoma patient, or renal cell carcinoma patient) comprising administering the PD-1 axis binding antagonist to the patient in combination with an IL-6 antagonist (e.g. an anti-IL6 receptor antibody, e.g. tocilizumab) in an amount effective to treat the cancer. The treatment optionally inhibits CD8⁺ T cell function. The cancer patient optionally has abnormal CRP and/or IL-6 level(s). The IL-6 antagonist is optionally administered prior to the PD-1 axis binding antagonist.

In another embodiment, the invention concerns a method of treating cancer (e.g. liver cancer, such as hepatocellular carcinoma, HCC) in a cancer patient comprising administering to the patient a combination of atezolizumab, bevacizumab, and tocilizumab in an amount effective to treat the cancer.

### BRIEF DESCRIPTION OF THE DRAWINGS

FIGS. 1a-i. depict plasma IL-6 and clinical outcomes in metastatic triple negative breast cancer (mTNBC), metastatic renal cell carcinoma (mRCC), and metastatic urothelial bladder carcinoma (mUC). (FIG. 1a) Plasma IL-6 (plasma IL-6) concentrations in healthy individuals and patients with mTNBC (P=1.87×10⁻⁶), mRCC (P=4.93×10⁻⁷), or mUC (P=1.35×10⁻⁷), compared using two-sided Mann-Whitney U-tests with Benjamini-Hochberg correction. (FIG. 1b) Pearson Correlation of plasma CRP with plasma IL-6 in patients with mTNBC, mRCC, or mUC. (FIG. 1c) Plasma IL-6 concentration in mTNBC patients who experienced complete response (CR), partial response (PR), stable disease (SD), or progressive disease (PD) following treatment with atezolizumab (compared using Kruskal-Wallis test). (FIGS. 1d-f) Association of high baseline plasma IL-6 with poor OS in mTNBC patients from PCD4989g (FIG. 1d), mRCC patients from IMmotion150 (FIG. 1e), and mUC patients from IMvigor210 (FIG. 1f). (FIG. 1g) Association of high baseline plasma IL-6 with poor OS in mUC patients treated with atezolizumab or with chemotherapy (IMvigor211). (FIG. 1h) Association of OS with on-treatment increase in plasma IL-6 (ratio between week 6 concentration and pre-treatment concentration (cutoff 1.05)) in IMvigor211. In panels FIGS. 1d-h, all HR values (with 95% CI in parentheses) are corrected in multivariate analysis as follows: ECOG (Eastern Cooperative Oncology Group) performance status, liver metastasis, and line of therapy in mTNBC; ECOG performance status and presence of liver metastasis in mUC; and MSKCC (Memorial Sloan Kettering Cancer Centre) prognostic risk score, previous nephrectomy, and liver metastasis in mRCC. (FIG. 1i) Single-cell RNA sequencing analysis of pre-treatment PBMCs from mUC patients (IMvigor210) with low (n=10) or high (n=10) plasma IL-6. Differential gene expression between CD8⁺ T cells from each patient group is shown.

FIGS. 2a-d depict tumor IL-6 gene expression and clinical outcomes in metastatic renal cell carcinoma (mRCC). (FIG. 2a) In situ hybridization (ISH) staining of IL6 mRNA in representative histological sections of mRCC tumors. An example of epithelial-restricted expression is shown in the left panel, and mixed epithelial and stromal staining in the right panel. Black arrows indicate representative epithelial cell expression and arrowheads indicate representative stromal cell expression. (FIG. 2b) Proportion of tumors with low/negative IL6 expression (staining in <1% of cells) or positive expression in epithelial cells only (yellow), stromal cells only (blue), or both epithelial and stromal cells (red). (FIG. 2c) Association of high IL6 expression and overall survival (OS) in the atezolizumab (left panel), atezolizumab+bevacizumab (middle panel), and sunitinib (right panel) treatment arms from IMmotion150. (FIG. 2d) Association of high IL6 expression and OS in patients with high tumor T cell signature expression from IMmotion150. In FIG. 2c and FIG. 2d, HR values (with 95% CI in parentheses) are adjusted after multivariate analysis including MSKCC (Memorial Sloan Kettering Cancer Centre) prognostic risk score, previous nephrectomy, and liver metastasis in mRCC.

FIGS. 3a-j depict suppression of CD8⁺ T cell effector function by IL-6. (FIGS. 3a-b) Splenocytes from VVT or Il6r^{-/-} mice were cultured with anti-CD3/CD28 antibodies and CD8⁺ T cells were analyzed by flow cytometry. Data represent one of 3 independent experiments (n=4 replicates per group). (FIG. 3a) Boolean analysis of IFN-y, TNF, and GzmB co-expression, with specific frequencies (+/−s.e.m) indicated separately for IFN-γ⁺ TNF⁺ GzmB⁺ cells. (FIG. 3b) Frequency (mean+/−s.e.m.) of IFN-γ⁺ cells, compared using one-way ANOVA with Tukey's multiple comparisons test (df=30). (FIG. 3c) CD8⁺ T cells isolated from C57BL/6J splenocytes, activated with anti-CD3/CD28 antibodies (+/−IL-6), and analyzed by flow cytometry. Data represent >4 independent experiments with n=4 replicates per group, compared using Student's t-tests (df=6; t=18.15 (% IFN-γ⁺); t=7.97 (% TNF⁺); t=8.62 (% IFN-γ⁺ TNF⁺)). (FIG. 3d) CD8⁺ T cells from Cd4.Cre×Stat3wmAit or Cd4.Cre×Stat3^{loxp/loxp} mice activated with anti-CD3/CD28 antibodies and analyzed by flow cytometry on day 3. Data represent one of 3 independent experiments (n=4 replicates per group), with pre-specified pairs compared using one-way ANOVA with Sidak's multiple comparisons test (df=18). (FIGS. 3e-j) OT-I splenocytes were activated as shown (FIG. 3e) and analyzed on day 7 by flow cytometry, cytotoxicity assay, or RNA-sequencing. (FIG. 3e) Mean (+/−s.e.m) frequency of IFN-γ, TNF, and GzmB co-expression in CD8⁺ T cells from one of 3 independent experiments (n=4 replicates per condition). Groups compared using one-way ANOVA with Tukey's multiple comparisons test (df=9). (FIG. 3f) Killing of SIINFEKL-pulsed MC38-GFP cells (“SIINFEKL” disclosed as SEQ ID NO: 34) by OT-I CD8⁺ T cells (5:1 T cell to target ratio). Data represent mean+/−s.e.m. from one of 3 independent experiments, with n=4 replicates per condition, compared using two-way ANOVA with Tukey's multiple comparisons test (df=9). (FIGS. 3g-h) OT-I cells were activated with or without IL-6, hyper-IL-6, isotype control antibody, or anti-IL6R antibody. CD8⁺ T cells were then FACS-sorted and analyzed by RNA-sequencing. (FIG. 3g) Principal components analysis. (FIG. 3h) Selected differentially expressed genes (FDR<0.05) associated with CD8⁺ T cell effector differentiation. (FIGS. 3i-j) Analysis of Eomes, Tbet, TCF1, and CD62L expression by OT-I cells activated with or without IL-6 by flow cytometry. (FIG. 3i) Boolean co-expression analysis. (FIG. 3j) Mean (+/−s.e.m.) frequencies of Eomes+Tbet+CD62L⁻ cells (effector-like), Eomes⁻ Tbet⁻ CD62L+ cells (naïve-like), and cells expressing TCF1 (a marker of stem-cell memory potential). Data are representative of 3 independent experiments with n=4 replicates per group, compared using Student's t-tests (df=6; from left to right, t=7.71, 5.78, and 1.15).

FIGS. 4a-k depict combination blockade of PD-L1 and IL6R in vivo. (FIGS. 4a-c) C57BL/6J mice were immunized as shown (panel a). After 7 days, splenocytes were stimulated with PMA (phorbol myristate acetate)/ionomycin and cytokine expression by CD8⁺ OT-I T cells was assessed by flow cytometry. (FIG. 4b) Detection of CD90.1⁺ OT-I cells among total CD8⁺ T cells (upper row), and expression of IFN-γ and GzmB by OT-I cells after restimulation (bottom row). (FIG. 4c) Total polyfunctional (GzmB⁺ IFN-γ⁺ TNF⁺) OT-I cells. Data are from n=10 mice per group (displayed as ratios relative to non-immunized/naive controls) and represent 3 independent experiments. Groups were compared using one-way ANOVA with Dunnett's multiple comparisons test (df=28). (FIG. 4d) Experimental design for IL6R and PD-L1 blockade in the orthotopic EMT6 breast cancer model. For immune pharmacodynamic (PD) studies (FIG. 4e-i), mice were sacrificed after 11 days; for therapeutic efficacy experiments (FIG. 4j-k), treatment was stopped at day 21 and mice were followed to day 50. (FIG. 4e) Plasma IL-6 concentration in healthy or tumor-bearing mice. Data indicate mean+/−s.e.m., pooled from 3 independent experiments, compared using Student's t-test (df=28; t=3.35). (FIG. 4f) Composition of CD45⁺ TIL (representative of 3 independent experiments). (FIG. 4g) Frequencies of CD8⁺ T cells, conventional Foxp3⁻ CD4⁺ T cells (Tconv), and regulatory Foxp3+ CD4⁺ T cells (Treg) among total CD45⁺ TIL. Data are pooled from 3 independent experiments, compared using one-way ANOVA with Dunnett's multiple comparisons test (df=63). (FIG. 4h) IFN-γ, TNF, and GzmB expression in tumor-infiltrating CD8⁺ T cells. (FIG. 4i) Frequency of polyfunctionality among tumor-infiltrating CD8⁺ T cells (left), their relative abundance in tumor tissue (normalized to tumor weight and scaled to the isotype control group; middle), and their abundance relative to Treg (right). Data are pooled from 3 independent experiments and compared using one-way ANOVA with Dunnett's multiple comparisons test (df=61). (FIG. 4j) Volumes of individual tumors from one of three independent experiments. Day 0 indicates start of treatment. CR, complete response; PR, partial response; PD, progressive disease. (FIG. 4k) Progression-free survival (PFS), defined as a 5× increase in tumor volume following treatment initiation. Data are pooled from 3 independent studies (n=10 mice per group). HR and P-values calculated using log-rank tests.

FIG. 5 depicts study profile of PCD4989g (mTNBC cohort), IMvigor210, IMvigor211, and IMmotion150 clinical trials. Flowchart showing number of intent-to-treat (ITT) patients in PCD4989g (mTNBC cohort), IMvigor210, IMvigor211 and IMmotion150, as well as the numbers of patients whose plasma or tumor RNAseq samples were included for analysis.

FIGS. 6a-b depict determination of a cut-off value to define high plasma IL-6. (FIG. 6a) Distribution of plasma IL-6 in healthy adult and mTNBC patients in PCD4989g, mUBC patients in IMvigor 210 and IMvigor 211, and mRCC patients in IMmotion150. (FIG. 6b) Plasma IL-6 values were transformed into normality using Box-Cox transformation, and pIL-6 values were derived at the stated standard deviations and confidence intervals from the pIL-6 distribution of healthy adults. Based on this analysis, a concentration of 0 pg/ml was chosen for downstream analyses as the definition of high pIL-6 status.

FIG. 7 depicts association of pIL-6 with objective response in mUBC. Rates of CR (complete response), PR (partial response), SD (stable disease), and PD (progressive disease) in patients with low or high pIL-6 in the IMvigor210 trial.

FIGS. 8a-c depict correlation of plasma CRP with OS. Association of high baseline plasma CRP (>3 mg/L) with poor OS in atezolizumab-treated mTNBC patients from PCD4989g (FIG. 8a), mUC patients from IMvigor210 (FIG. 8b), and mRCC patients from IMmotion150 (FIG. 8c).

FIGS. 9a-h depict single-cell RNA-sequencing analysis of PBMCs from mUC patients with high or low plasma IL-6. (FIG. 9a) UMAP clustering of cells pooled from plasma IL-6-low (n=10) and plasma IL-6-high (n=10) patients to identify cell populations. (FIG. 9b) Expression of diagnostic lineage genes in the UMAP-organized cell clusters. Co-expression of CD3D and CD8A identify clusters 4 and 7 as CD8⁺ T cells. (FIG. 9c) Number of cells in each cluster. (FIG. 9d) Number of transcripts per cell identified in each cluster. (FIG. 9e) Distribution of cells originating from plasma IL-6-low patients (yellow) and plasma IL-6-high patients (blue) across clusters. (FIG. 9f) Fraction of each cluster that is comprised of cells from plasma IL-6-low patients (yellow) or plasma IL-6-high patients (blue). (FIG. 9g) Distribution of cells from individual patients across cell clusters. (FIG. 9h) Cellular contribution from individual donors to each UMAP cluster.

FIGS. 10a-d depict effects of IL-6 on CD8⁺ T cell activation. (FIGS. 10 a-b) CD8⁺ T cell proliferation (day 3) in response to anti-CD3/CD28 stimulation with or without recombinant IL-6 or hyper-IL-6. Cell-tracer dilution plots are shown in FIG. 10a, and associated proliferation index in FIG. 10b. Data are representative of 3 independent experiments with n=4 replicates per group. (FIG. 10c) IFN-γ and TNF expression by mouse CD8+ T cells isolated from splenocytes and activated with anti-CD3/CD28 antibodies for 3 days with or without recombinant IL-6, IL-2, or IL-15/IL-15RA complex. (FIG. 10d) IFN-γ expression (left) and viability relative to cytokine-free controls (right) from cells cultured as described for FIG. 10c. Data are mean+/−s.e.m. of 4 technical replicates and are representative of 4 independent experiments (assessing the effect of IL-2) and one of two independent experiments (assessing the effect of IL-15). Pre-specified pairs (−IL-6 vs+IL-6) were compared using one-way ANOVA with Sidak's multiple comparisons test (df=24). **P=0.0039, ***P<0.0001.

FIGS. 11a-b depict effect of IL-6 on naïve and memory CD8⁺ T cell activation. (FIG. 11a) Naïve and memory CD8⁺ T cells were FACS-purified from splenocytes of wild type C57BL/6J mice, activated with anti-CD3/CD28 antibodies in the presence or absence of IL-6 or hyper-IL-6, and assessed for effector function by flow cytometry on day 3. (FIG. 11b) Frequency of CD8⁺ T cells co-expressing IFN-γ, TNF, and GzmB. Data indicate mean+/−s.e.m. from n=4 replicates, and are representative of two independent experiments. Groups are compared using one-way ANOVA with Tukey's multiple comparisons tests (df=9).

FIGS. 12a-b depict effect of IL-6 on CD8⁺ T cell cytotoxicity. OT-I splenocytes were incubated for 2 days with SIINFEKL peptide (SEQ ID NO:34) in the presence or absence of recombinant IL-6 or hyper-IL-6. Cells were then maintained with IL-2 alone for 3 days before co-culture with MC38-GFP cells that express ovalbumin (FIG. 12a) or were pulsed with SIINFEKL peptide (SEQ ID NO:34) (FIG. 12b). In FIG. 12a, Cells were cultured at a 20:1 T cell to target ratio; target cell lysis was detected by loss of GFP+ nuclei over time, and normalized at each timepoint to MC38 cells cultured without T cells. Comparisons with the control group were made using 2-way ANOVA with Tukey's multiple comparisons test (df=9). In FIG. 12b, SIINFEKL-pulsed MC38-GFP cells (SEQ ID NO:34) were incubated with increasing T cell ratios as indicated for 12 hours. Data points represent mean+/−s.e.m. from n=4 replicates. Data are representative of 3 independent experiments.

FIGS. 13a-c depict transcriptomic effects of IL-6 signaling in CD8⁺ T cells. OT-I splenocytes were incubated for 2 days with SIINFEKL peptide (SEQ ID NO:34) in the presence or absence of recombinant IL-6, hyper-IL-6, isotype control IgG, or anti-IL6R antibody. Cells were then maintained with IL-2 alone for a further 3 days before re-stimulation with anti-CD3 and anti-CD28 antibodies. Live CD8⁺ T cells were purified by FACS on day 7 and analyzed by RNA-sequencing. (FIG. 13a) Volcano plots of differential gene expression in all possible pairwise comparisons. Numbers of differentially expressed genes refer to those with an absolute fold-change >2 (log 2 fold change >1) and FDR (false discovery rate) <0.05. (cytokines/receptors; cytoxicity; chemokines/homing) (FIG. 13b-1); transcripton factors; stimulation/co-inhibition (FIG. 13b-2) Heat maps of genes that were significantly differentially expressed (FDR<0.05) between cells treated with anti-IL6R versus IL-6 or hyper-IL-6. Selected genes are organized into separate heat maps according to function. (c) Gene ontology analysis of genes differentially expressed between cells treated with anti-IL6R versus IL-6 or hyper-IL-6. Genes with FDR<0.05 and log 2 fold change ≤−1 or ≥1 were selected for analysis. Significantly enriched GO terms (FDR<0.05) are plotted by fold enrichment on the x-axis, and FDR on the y-axis.

FIGS. 14a-c depict impact of IL-6R and PD-L1 blockade on CD8⁺ T cell activation in vivo. (FIG. 14a) Bulk splenocytes from wild type C57BL/6J mice were stimulated with anti-CD3 and anti-CD28 antibodies for 3 days with or without the indicated blocking antibodies. Cytokine expression (gated on CD8⁺ T cells) was assessed by flow cytometry. Data are mean+/−s.e.m. from n=4 replicate samples, compared by one-way ANOVA with Dunnett's multiple comparisons tests with control samples as the reference group (df=12). (FIGS. 14b-c) 0.5×106 naïve CD8⁺ OT-I T cells (Thy1.1+) were adoptively transferred into wild type C57BL/6J mice (Thy1.2+). Mice were then treated with isotype control, anti-IL6R, or anti-PD-L1 antibodies and immunized intravenously with DEC-OVA (50 μg/kg) and agonistic anti-CD40 antibody (2.5 mg/kg). Splenocytes were isolated after 7 days, restimulated with PMA/ionomycin, and evaluated for effector function by flow cytometry. Data shown are gated on Thy1.1+CD8⁺ T cells (OT-I cells). (FIG. 14b) Total viable OT-I cells prior to restimulation. (FIG. 14c) Frequency of OT-I cells co-expressing IFN-γ, TNF, and GzmB after restimulation. Data in FIGS. 14b and 14c are mean+/−s.e.m. from n=10 mice per group from one of four independent experiments. Groups were compared using one-way ANOVA with Dunnett's multiple comparisons tests (df=38), using combination-treated mice as the reference group.

FIGS. 15a-b depict effect of IL-6 on EMT6 cell growth in vitro. (FIG. 15a) Activation of STAT3 (assessed by detection of p-STAT3 Y705; MSD assay) after 15 minutes of treatment with IL-6 or hyper-IL-6. Values are normalized to untreated cells. (FIG. 15b) Longitudinal measurement of EMT6 cell confluence (Incucyte live-cell analysis). Data points are mean+/−s.e.m. from 3 experimental replicates.

FIGS. 16a-b depict immunological features of EMT6 tumor-bearing mice during anti-IL6R and/or anti-PD-L1 therapy. (FIG. 16a) Absolute abundance (normalized to tumor weight and scaled to the isotype control group) of tumor-infiltrating CD8⁺ T cells, Foxp3-conventional CD4⁺ T cells, and Foxp3+ regulatory T cells. Groups compared using one-way ANOVA with Dunnett's multiple comparisons test (df=63). (FIG. 16b) Frequencies of leukocyte populations among total tumor-infiltrating CD45+ cells (top row) and their absolute abundance normalized to tumor weight and scaled to the isotype control group (lower row). Data are pooled from 3 independent experiments, with n=16-17 mice per group, compared using one-way ANOVA with Dunnett's multiple comparisons test (df=63).

FIGS. 17a-c depict peripheral assessment of immune activation in EMT6 tumor-bearing mice treated with anti-IL6R and anti-PD-L1. (FIG. 17a) Plasma cytokine concentrations after 11 days of treatment, measured via Luminex profiling (Millipore). Bars indicate mean+/−s.e.m. from n=9-10 mice per group. Groups compared using one-way ANOVA with Dunnett's multiple comparisons test (df=34). (FIG. 17b-c) CD8⁺ T cells were harvested from tumor-draining lymph nodes and re-stimulated ex vivo with PMA/ionomycin before analysis by flow cytometry. (FIG. 17b) Representative plots depicting expression of GzmB and IFN-γ in CD8⁺ T cells. (FIG. 17c) Frequency of GzmB and IFN-γ co-expression. Bars indicate mean+/−s.e.m. from n=5-6 mice per group, representative of 3 independent experiments. Groups compared using one-way ANOVA with Dunnett's multiple comparisons test (df=18).

FIGS. 18a-e depict immunostimulatory activity of anti-IL6R/anti-PD-L1 combination therapy in subcutaneous CT26 tumors. BALB/c mice with established (130-250 mm3) CT26 tumors were treated with antibodies against IL6R, PD-L1, a combination of the two, or isotype control antibodies for 11-12 days before sacrifice. (FIG. 18a) Representative flow cytometry plots depicting GzmB and IFN-γ expression by re-stimulated tumor-infiltrating CD8⁺ T cells. (FIG. 18b) Frequency of polyfunctionality (co-expression of IFN-γ, TNF, and GzmB) in CD8⁺ tumor-infiltrating T cells following ex vivo stimulation with PMA and ionomycin, pooled from two independent experiments. Anti-PD-L1 and anti-PD-L1/IL6R groups compared using t-test (t=1.78; df=23). (FIG. 18c) Ratio of polyfunctional CD8⁺ T cells to Foxp3+ CD4+ regulatory T cells, pooled from two independent experiments. Anti-PD-L1 and anti-PD-L1/IL6R groups compared using t-test (t=2.06; df=23). (FIG. 18d) Kaplan-Meier progression-free survival analysis (defined as a 5× increase in tumor volume) of the all experimental groups (left), anti-PD-L1 vs isotype control (middle), and anti-IL6R/PD-L1 vs anti-PD-L1 (right), pooled from two independent experiments. HR and P-values calculated using the log-rank test. (FIG. 18e) Resected tumor weights at the time of sacrifice, pooled from two independent experiments. Anti-PD-L1 and anti-PD-L1/IL6R groups compared using t-test (t=2.01; df=23).

FIG. 19 depicts working mechanistic model for IL6R and PD-L1 blockade synergy. The PD-1/PD-L1 axis is a major inhibitor of CD8⁺ T cell activation via repression of TCR (“signal 1”) and CD28 (“signal 2”) signaling. Under in vitro experimental conditions, PD-1/PD-L1 signaling is limited, T cell activation is efficient, and the ability of IL-6 signaling to inhibit effector function (a form of “signal 3”) is readily apparent. However, PD-1/PD-L1 signaling is a dominant checkpoint on T cell activation in vivo; in this context, IL-6 has only a modest influence on T cell effector function due to PD-1/PD-L1-mediated blockage of TCR and CD28. When PD-1/PD-L1 signaling is neutralized, T cell activation is enhanced but acquisition of effector function remains limited due to IL-6. Combined blockade of PD-1/PD-L1 and IL-6 signaling allows both efficient TCR/CD28 signaling and development of cytotoxic effector function, leading to potent effector T cells with enhanced anti-tumor activity. The molecular mechanism by which IL-6 signaling impairs effector function is strictly dependent on STAT3, but remains to be fully elucidated.

FIG. 20 depicts demographic characteristics of patients in the IMvigor210 and IMvigor211 studies.

FIG. 21 depicts demographic characteristics of the patients in the IMmotion150 study.

FIG. 22 depicts demographic characteristics of the TNBC patients in the PCD4989g study.

FIG. 23 depicts schematically how the PD-L1 pathway downregulates the anticancer immune response during two steps within the cancer-immunity cycle.

FIG. 24 depicts clinical activity associated with atezolizumab monotherapy in patients with PD-L1-positive mTNBC.

FIG. 25 depicts biomarkers of systemic myeloid inflammation are associated to poor prognostic baseline characteristics in mTNBC.

FIG. 26 depicts correlation between plasma inflammatory biomarkers.

FIG. 27 depicts plasma inflammatory biomarkers are associated with increased neutrophils (FIG. 27a) and monocytes in peripheral blood (FIG. 27b).

FIG. 28 depicts how atezolizumab monotherapy responders have lower baseline levels of systemic biomarkers of inflammation.

FIG. 29 depicts improved PFS and OS with atezolizumab monotherapy in patients with reduced inflammatory systemic biomarkers.

FIG. 30 depicts multivariate analysis: baseline circulating IL-6/CRP axis, but not IL8, is associated with atezolizumab monotherapy OS in TNBC.

FIG. 31 depicts Increase of IL-6/CRP in mTNBC patients experiencing disease progression.

FIG. 32 depicts a possible mechanism of action: Systemic inflammation (IL-6/CRP) might reduce atezolizumab-induced T cell proliferation.

FIG. 33 depicts poor prognosis associated with elevated baseline IL-6/CRP axis regardless of treatment.

FIG. 34 depicts how dual PD-L1/IL6R blockade controls tumor growth in syngeneic EMT6 TNBC mouse model.

FIGS. 35a-c depict effect of IL-6 conditioning on CD8⁺ T cell effector function.

### DETAILED DESCRIPTION OF EMBODIMENTS OF THE INVENTION

### I. Introduction

The present invention provides therapeutic methods and compositions for cancer, including bladder cancer, urothelial carcinoma, kidney cancer, renal cell carcinoma, and breast cancer (e.g. triple-negative breast cancer) with a combination of a PD-1 axis binding antagonist (e.g. an anti-PD-L1 antibody such as atezolizumab) and an IL6 antagonist (e.g. an anti-IL6 receptor monoclonal antibody such as tocilizumab). In one embodiment, the cancer patient has CRP and/or IL-6 above the upper limit of normal and, optionally, also expresses PD-L1.

### II. Definitions

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist.

The terms “programmed death ligand 1” and “PD-L1” refer herein to a native sequence PD-L1 polypeptide, polypeptide variants, and fragments of a native sequence polypeptide and polypeptide variants (which are further defined herein). The PD-L1 polypeptide described herein may be that which is isolated from a variety of sources, such as from human tissue types or from another source, or prepared by recombinant or synthetic methods.

A “native sequence PD-L1 polypeptide” comprises a polypeptide having the same amino acid sequence as the corresponding PD-L1 polypeptide derived from nature.

A “PD-L1 polypeptide variant,” or variations thereof, means a PD-L1 polypeptide, generally an active PD-L1 polypeptide, as defined herein having at least about 80% amino acid sequence identity with any of the native sequence PD-L1 polypeptide sequences as disclosed herein. Such PD-L1 polypeptide variants include, for instance, PD-L1 polypeptides wherein one or more amino acid residues are added, or deleted, at the N- or C-terminus of a native amino acid sequence. Ordinarily, a PD-L1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence PD-L1 polypeptide sequence as disclosed herein. Ordinarily, PD-L1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 281, 282, 283, 284, 285, 286, 287, 288, or 289 amino acids in length, or more. Optionally, PD-L1 variant polypeptides will have no more than one conservative amino acid substitution as compared to a native PD-L1 polypeptide sequence, alternatively no more than 2, 3, 4, 5, 6, 7, 8, 9, or 10 conservative amino acid substitutions as compared to a native PD-L1 polypeptide sequence.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is atezolizumab, marketed as TECENTRIQ® with a WHO Drug Information (International Nonproprietary Names for Pharmaceutical Substances), Proposed INN: List 112, Vol. 28, No. 4, published Jan. 16, 2015 (see page 485) described herein. In another specific aspect, an anti-PD-L1 antibody is MDX-1105 described herein. In still another specific aspect, an anti-PD-L1 antibody is YW243.55.570 described herein. In still another specific aspect, an anti-PD-L1 antibody is MEDI4736 (durvalumab) described herein. In still another specific aspect, an anti-PD-L1 antibody is MSB0010718C (avelumab) described herein.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (pembrolizumab) described herein. In another specific aspect, a PD-1 binding antagonist is MEDI-0680 (AMP-514) described herein. In another specific aspect, a PD-1 binding antagonist is PDR001 described herein. In another specific aspect, a PD-1 binding antagonist is REGN2810 described herein. In another specific aspect, a PD-1 binding antagonist is BGB-108 described herein.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen-binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

Herein “human interleukin 6” (abbreviated as “IL-6”) is a cytokine also known as B cell-stimulating factor 2 (BSF-2), or interferon beta-2 (IFNB2), hybridoma growth factor, and CTL differentiation factor. IL-6 was discovered as a differentiation factor contributing to activation of B cells (Hirano et al., Nature 324: 73-76 (1986)), and was later found to be a multifunction cytokine which influences the functioning of a variety of different cell types (Akira et al., Adv. in Immunology 54: 1-78 (1993)). Naturally occurring human IL-6 variants are known and included in this definition. Human IL-6 amino acid sequence information has been disclosed, see for example, www.uniprot.org/uniprot/P05231.

For the purposes herein “human interleukin 6 receptor” (abbreviated as “IL-6R”) refers to the receptor which binds IL-6, including both membrane-bound IL-6R (mIL-6R) and soluble IL-6R (sIL-6R). IL-6R can combine with interleukin 6 signal transducer glycoprotein 130 to form an active receptor complex. Alternatively spliced transcript variants encoding distinct isoforms of IL-6 have been reported and are included in this definition. The amino acid sequence structure of human IL-6R and its extracellular domain have been described; see, for example, Yamasaki et al., Science, 241: 825 (1988).

A “neutralizing” anti-IL-6R antibody herein is one which binds to IL-6R and is able to inhibit, to a measurable extent, the ability of IL-6 to bind to and/or active IL-6R. Toclizumab is an example of a neutralizing anti-IL-6R antibody.

“Tocilizumab” or “TCZ” is a recombinant humanized monoclonal antibody that binds to human interleukin-6 receptor (IL-6R). It is an IgG1 K (gamma 1, kappa) antibody with a two heavy chains and two light chains forming two antigen-binding sites. In a preferred embodiment, the light chain and heavy chain amino acid sequences of Tocilizumab comprise SEQ ID NOs. 32 and 33.

The term “biomarker” as used herein refers to an indicator, e.g., predictive, diagnostic, and/or prognostic, which can be detected in a sample, for example, PD-L1, IL-6, and/or CRP biomarker(s). The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

A “level above the upper limit of normal” refers to an amount of a biomarker that is abnormal or atypical in a subject (including a healthy subject) or patient (including a cancer patient, e.g. with breast cancer, urothelial carcinoma, or renal cell carcinoma). Assays for measuring such abnormal amounts of CRP and/or IL-6 are disclosed herein, along with exemplary “cut-offs” or “comparator” amounts of CRP and/or IL-6 for identifying patients eligible for therapy.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing ora degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In one embodiment, the sample is a blood specimen from the patient (e.g. for a CRP and/or IL-6 bioassay).

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a liver tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8⁺ T lymphocytes and/or CD4⁺ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

In one embodiment, the sample comprises “tumor cells and/or tumor-infiltrating immune cells” from the patient (e.g. for a PD-L1 bioassay).

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

As used herein, “objective response rate” or “objective response rate” (ORR) refers to the sum of complete response (CR) rate and partial response (PR) rate. For example, in some embodiments, ORR refers to the proportion of patients with a confirmed objective response, either CR or PR, observed on two assessments greater than or equal to 28 days apart per RECIST v1.1, based on investigator assessment.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease. In some embodiments, PFS may be defined as the time from randomization or the beginning of treatment to the first documented disease progression as assessed by RECIST v1.1, or death from any cause, whichever occurs first. PFS of a combination of PD-1 axis binding antagonist and IL6 antagonist can be compared to the PFS without the IL6 antagonist (e.g. compared with PD-1 axis binding antagonist alone).

As used herein, “overall survival” (OS) refers to the percentage of individuals in a group who are likely to be alive after a particular duration of time. OS of a combination of PD-1 axis binding antagonist and IL6 antagonist can be compared to the OS without the IL6 antagonist (e.g. compared with PD-1 axis binding antagonist alone).

As used herein, the term “duration of response” (DOR) refers to a length of time from documentation of a tumor response until disease progression or death from any cause, whichever occurs first.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer (e.g., breast cancer, urothelial carcinoma, or renal cell carcinoma) are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

An “effective amount” or “therapeutically effective amount,” as used interchangeably herein, is at least the minimum amount required to effect a measurable improvement or prevention of a particular disorder. An effective amount herein may vary according to factors such as the disease state, age, sex, and weight of the patient, and the ability of the agent to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, and enhancing effect of another medication such as via targeting, delaying the progression of the disease, and/or prolonging survival. In the case of a cancer or a tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

For a combination, “an amount effective to treat the cancer” comprises amounts of each of the components of the combination that treat the cancer patient. Such amounts may comprise standard dosages of each component (or may be lowered in the combination therapy regimen). In one embodiment, the “amount effective” of the combination achieves a clinical response greater than treatment with either agent alone, greater than PD-1 axis binding antagonist (e.g. anti-PD-L1 antibody such as atezolizumab) alone, greater than treatment without IL6 antagonist (e.g. without anti-IL6 receptor antibody or without tocilizumab); or greater than treatment with PD-1 axis binding antagonist (e.g. anti-PD-L1 antibody such as atezolizumab) and chemotherapy (without the IL6 antagonist). The amount effective of the combination may inhibit CD8⁺ T cell function and/or reduce or prevent therapeutic resistance to the PD-1 axis binding antagonist.

The terms “cancer” and “cancerous” refer to or describe the physiological condition in mammals that is typically characterized by unregulated cell growth. The term “bladder cancer” includes, but is not limited to, UC, and which may be, for example, locally advanced or metastatic. The methods described herein are suitable for treatment of various stages of cancer, including cancers that are locally advanced and/or metastatic. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV).

The term “upper tract UC” refers to UC of the renal pelvis or ureter. The upper tract UC may be upper tract metastatic UC. A minority of cases (e.g., about 5-10%) of UC are upper tract UC.

The term “lower tract UC” refers to UC of the bladder or urethra. The lower tract UC may be lower tract metastatic UC. The majority of cases (e.g., about 90-95%) of UC are lower tract UC.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one embodiment, the cytotoxic agent is a platinum-based chemotherapeutic agent. In one embodiment, the cytotoxic agent is an antagonist of EGFR. In one embodiment the cytotoxic agent is N-(3-ethynylphenyl)-6,7-bis(2-methoxyethoxy)quinazolin-4-amine (e.g., erlotinib, TARCEVA®). In one embodiment the cytotoxic agent is a RAF inhibitor. In one embodiment, the RAF inhibitor is a BRAF and/or CRAF inhibitor. In one embodiment the RAF inhibitor is vemurafenib. In one embodiment, the cytotoxic agent is a PI3K inhibitor.

As used herein, the term “chemotherapeutic agent” includes compounds useful in the treatment of cancer, such as bladder cancer (e.g., UC, including locally advanced or metastatic UC). Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, rapamycin (Sirolimus, RAPAMUNE®, Wyeth), lapatinib (TYKERB®, GSK572016, Glaxo Smith Kline), lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxanes; chloranmbucil; GEMZAR® (gemcitabine); 6-thioguanine; mercaptopurine; methotrexate; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, and derivatives of any of the above.

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin.

A “platinum-based chemotherapy,” as used herein, refers to a chemotherapy regimen that includes a platinum-based chemotherapeutic agent. For example, an IL6 antagonist may include a platinum-based chemotherapeutic agent (e.g., cisplatin or carboplatin) in combination with one or more additional chemotherapeutic agents, e.g., a nucleoside analog (e.g., gemcitabine).

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids, and derivatives of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant exclusively human-sequence, full-length IgG₁ λ antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533) and variants thereof, such as chimerized 225 (C225 or Cetuximab; ERBUTIX®) and reshaped human 225 (H225) (see, e.g., WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al., Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[(4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER-targeted tyrosine kinase inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (imatinib mesylate). Anti-angiogenesis agents also include native angiogenesis inhibitors, e.g., angiostatin, endostatin, etc. See, for example, Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and, Sato Int. J. Clin. Oncol., 8:200-206 (2003).

The terms a “subject” or “patient” refer to a human subject or human patient.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in ImmunoL 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or nonhuman primate having the desired specificity, affinity, and/or capacity. In some embodiments, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The term “package insert” is used to refer to instructions customarily included in commercial packages of therapeutic products, that contain information about the indications, usage, dosage, administration, combination therapy, contraindications and/or warnings concerning the use of such therapeutic products.

The terms “pharmaceutical formulation” and “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered. Such formulations are sterile.

In a preferred embodiment, the pharmaceutical composition or pharmaceutical formulation is administered to a human subject.

A “sterile” pharmaceutical formulation is aseptic or free or essentially free from all living microorganisms and their spores.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “in combination with” or “in conjunction with” refers to administration of one treatment modality in addition to another treatment modality, for example, a treatment regimen that includes administration of a PD-1 axis binding antagonist (e.g., an anti-PD-L1 antibody such as atezolizumab) and a IL6 antagonist (e.g., an anti-IL6 receptor antibody such as tocilizumab). As such, “in conjunction with” refers to administration of one treatment modality before, during, or after administration of the other treatment modality to the individual.

As used herein, the term “bevacizumab” refers to an anti-vascular endothelial growth factor (VEGF) antagonist antibody comprising the heavy and light chain sequences disclosed, inter alia, in CAS Registry Number 216974-75-3.

### III. Therapeutic Methods and Compositions

Provided herein are methods for treating cancer, including, bladder cancer, urothelial carcinoma (including locally advanced or metastatic UC), breast cancer (including triple negative breast cancer), and kidney cancer (including renal cell carcinoma) in a patient comprising administering to the patient a treatment regimen comprising an effective amount of combination of a PD-1 axis binding antagonist (e.g., an anti-PD-L1 antibody, such as atezolizumab) and an IL6 antagonist (e.g. an anti-IL6 receptor antibody, such as tocilizumab).

In a first aspect, the invention concerns a method of treating a cancer patient comprising administering to the patient a combination of an IL-6 antagonist and a PD-1 axis binding antagonist in an amount effective to treat the cancer.

In one embodiment, the cancer is a breast cancer, a bladder cancer, a kidney cancer, a liver cancer, a lung cancer, a colorectal cancer, an ovarian cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In one embodiment, the cancer is not melanoma or pancreatic cancer.

In one embodiment, the cancer is breast cancer, such as triple negative breast cancer (TNBC).

In one embodiment, the cancer is bladder cancer.

In one embodiment, the cancer is urothelial carcinoma.

In one embodiment, the cancer is kidney cancer.

In one embodiment, the cancer is renal cell carcinoma.

In one embodiment, the cancer is hepatocellular carcinoma.

The patient to be treated herein may have been subjected to one or more assays, more detail about such assays being provided in Section VII. below.

In one embodiment, the patient has C-reactive protein (CRP) level above the upper limit of normal. For example, the patient may have 3 mg/L CRP, e.g. mg/L CRP. Various assays for measuring CRP are available. In one embodiment, the CRP is measured by enzyme-linked immunosorbent assay (ELISA) assay and the sample is a blood sample from the patient.

In one embodiment, the patient has IL-6 level above the upper limit of normal. For example, the patient may have ≥10 pg/mL IL-6, e.g. pg/mL IL-6. Various assays are available for measuring IL-6. In one embodiment, IL-6 is measured by enzyme-linked immunosorbent assay (ELISA) assay and the sample is a blood sample from the patient.

In one embodiment, the patient expresses PD-L1. For example, the patient may have PD-L1 stained tumor cells (TC) and/or tumor-infiltrating immune cells (IC), e.g. where the PD-L1 stained TC and/or IC cover ≥1% of the tumor area, e.g. ≥5% of the tumor area.

In one embodiment, the patient has CRP above the upper limit of normal and the patient's tumor expresses PD-L1.

In one embodiment, the patient has IL-6 above the upper limit of normal and the patient's tumor expresses PD-L1.

In on embodiment, the patient has both CRP and IL-6 above the upper limit of normal.

In one embodiment, the patient has both CRP and IL-6 above the upper limit of normal and the patient's tumor expresses PD-L1.

In one embodiment, the patient's tumor expresses PD-L1 (e.g. mTNBC).

In one embodiment, CRP and/or IL-6 is evaluated prior to treatment and the patient has elevated CRP and/or IL-6 prior to treatment.

In one embodiment, CRP and/or IL-6 is evaluated during treatment or following treatment and the patient has elevated CRP and/or IL-6.

In one embodiment, the patient fails to respond or has unacceptable toxicity to a prior therapy e.g. where the prior therapy is therapy with a PD-L axis binding antagonist (e.g. an anti-PD-L1 antibody such as atezolizumab) without the IL-6 antagonist (e.g. an anti-IL6 receptor antibody such as tocilizumab), and CRP and/or IL-6 is measured in the patient to evaluate whether combination therapy as disclosed herein should be used.

In one embodiment, the IL-6 antagonist is administered to the patient prior to the administration of the PD-1 axis binding antagonist.

In one embodiment, the patient does not have cytokine release syndrome (CRS).

In one embodiment, the IL-6 antagonist is an anti-IL6 receptor antibody, e.g. tocilizumab, satralizumab, sarilumab, N1-1201, or vobarilizumab, preferably tocilizumab.

In another embodiment, the IL-6 antagonist binds IL-6, e.g. it is selected from: siltuximab, sirukumab, olokizumab, clazakizumab, EBI-031, and olamkicept.

More detail about IL-6 antagonists is provided in Section V. below.

In one embodiment, the PD-L1 axis binding antagonist is a PD-L1 binding antagonist, a PD-1 binding antagonist, or a PD-L2 binding antagonist.

In one embodiment, the PD-L1 axis binding antagonist is a PD-L1 binding antagonist, e.g. which inhibits the binding of PD-L1 to both PD-1 and B7-1 and/or is an antibody.

Examples of PD-L1 binding antibodies contemplated herein include atezolizumab, MDX-1105, MEDI4736 (durvalumab), or MSB0010718C (avelumab), atezolizumab being preferred.

In another embodiment, the PD-L1 axis binding antagonist is a PD-1 binding antagonist, examples of which include: MDX-1106 (nivolumab), MK-3475 (pembrolizumab), MEDI-0680 (AMP-514), PDR001, REGN2810, BGB-108, and AMP-224.

More detail about PD-1 axis binding antagonists is provided in Section IV. below.

In one embodiment, the IL-6 antagonist is an IL-6 receptor binding antibody (e.g. tocilizumab) and the PD-1 axis binding inhibitor is a PD-L1 binding antibody (e.g. atezolizumab).

In one embodiment, tocilizumab is administered by intravenous (iv) infusion at a dose of 8 mg/kg every 4 weeks (Q4w) on Day 1 of each 28-day cycle, e.g. administered until disease progression or unacceptable toxicity.

In one embodiment, atezolizumab is administered intravenously (iv) at a fixed dose of 840 mg every 2 weeks (Q2W) on Days 1 and 15 of each 28-day cycle, e.g. until disease progression or unacceptable toxicity.

In one embodiment, the tocilizumab is administered first and atezolizumab is administered after the tocilizumab administration. For example, the atezolizumab may be administered about two hours after the conclusion of the tocilizumab administration.

In one embodiment, treatment achieves an objective response rate (ORR), including a complete response (CR) and/or a partial response (PR).

In one embodiment, the treatment extends progression free survival (PFS) and/or overall survival (OS), e.g. to a greater extent than treatment without the IL-6 antagonist.

In one embodiment, the treatment results in an increased abundance of CD8⁺ T cells in the patient relative to that of a subject who has not been administered the IL-6 antagonist.

In one embodiment, the treatment reduces or prevents therapeutic resistance to the PD-1 axis binding antagonist.

In another embodiment, the invention concerns a method of treating a cancer patient comprising administering to the patient a combination of an anti-IL6 receptor antibody and an anti-PD-L1 antibody in an amount effective to treat the cancer. For example, the cancer can be breast cancer, bladder cancer, or renal cell carcinoma.

In yet another embodiment, the invention provides a method of treating a cancer patient with high C-reactive protein (CRP) level comprising administering to the patient a combination of an anti-IL6 receptor antibody and an anti-PD-L1 antibody in an amount effective to treat the cancer.

In another embodiment, the invention concerns a method of treating advanced urothelial carcinoma in a cancer patient comprising administering to the patient a combination of tocilizumab and atezolizumab in an amount effective to treat the cancer.

In another embodiment, the invention concerns a method of treating triple negative breast cancer (TNBC) in a cancer patient comprising administering to the patient a combination of tocilizumab, atezolizumab, and chemotherapy (e.g. a taxane such as Nab paclitaxel) in an amount effective to treat the cancer.

Exemplary dosages for tocilizumab include 8 mg/kg or 4 mg/kg (by intravenous infusion) and, alternatively, 162 mg administered by subcutaneous administration.

In any of the preceding examples, each dosing cycle may have any suitable length, e.g., about 7 days, about 14 days, about 21 days, about 28 days, or longer. In one embodiment, each dosing cycle is about 28 days.

Any suitable number of dosing cycles may be used, e.g., 1, 2, 3, 4, 5, 6, 7, 8, 9, 10, 11, 12, 13, 14, 15, 20, 25, 30, 35, 40, 45, 50, or more dosing cycles. In some embodiments, 10 or fewer dosing cycles may be used. In some embodiments, 20 or fewer dosing cycles are used. In some embodiments, 25 or fewer dosing cycles are used. In one embodiment, the combination is administered until disease progression or unacceptable toxicity.

In some embodiments, the PD-L1 binding antagonist is an anti-PD-L1 antibody. Any suitable anti-PD-L1 antibody described herein or known in the art may be used. In some embodiments, the anti-PD-L1 antibody is selected from the group consisting of atezolizumab (TECENTRIQ®), MDX-1105, MEDI4736 (durvalumab), and MSB0010718C (avelumab).

Atezolizumab may be administered to the subject at any suitable dosage. In some embodiments, atezolizumab is administered to the subject intravenously at a dose of about 840 mg every 2 weeks, about 1200 mg every 3 weeks, or about 1680 mg of every 4 weeks. In some embodiments, atezolizumab is administered to the subject intravenously at a dose of about 840 mg every 2 weeks. In some embodiments, atezolizumab is administered to the subject in a 28-day dosing cycle.

In other embodiments, the PD-1 axis binding antagonist is a PD-1 binding antagonist. In some embodiments, the PD-1 binding antagonist inhibits the binding of PD-1 to one or more of its ligand binding partners. In some embodiments, the PD-1 binding antagonist inhibits the binding of PD-1 to PD-L1, PD-L2, or both PD-L1 and PD-L2. In some embodiments, the PD-1 binding antagonist is an anti-PD-1 antibody. In some embodiments, the anti-PD-1 antibody is selected from the group consisting of: MDX-1106 (nivolumab), MK-3475 (pembrolizumab), MEDI-0680 (AMP-514), PDR001, REGN2810, and BGB-108. In some embodiments, the PD-1 binding antagonist is an Fc fusion protein. In some embodiments, the Fc fusion protein is AMP-224.

As a general proposition, the therapeutically effective amount of a PD-1 axis binding antagonist (e.g., an anti-PD-L1 antibody (e.g., atezolizumab) or an anti-PD-1 antibody) administered to a human will be in the range of about 0.01 to about 50 mg/kg of patient body weight, whether by one or more administrations. In some embodiments, for example, the antagonist (e.g., a PD-1 axis binding antagonist (e.g., an anti-PD-L1 antibody (e.g., atezolizumab) or an anti-PD-1 antibody)) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In some embodiments, the antagonist (e.g., a PD-1 axis binding antagonist (e.g., an anti-PD-L1 antibody (e.g., atezolizumab) or an anti-PD-1 antibody) and/or a VEGF antagonist (e.g., an anti-VEGF antibody (e.g., bevacizumab)) is administered at 15 mg/kg (e.g. every 3 weeks). However, other dosage regimens may be useful. In one embodiment, a PD-1 axis binding antagonist (e.g., an anti-PD-L1 antibody (e.g., atezolizumab) or an anti-PD-1 antibody) is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some embodiments, the antagonist (e.g., an anti-PD-L1 antibody (e.g., atezolizumab) or an anti-PD-1 antibody) may be administered at a dose of about 1000 mg to about 1400 mg every three weeks (e.g., about 1100 mg to about 1300 mg every three weeks, e.g., about 1150 mg to about 1250 mg every three weeks). In some embodiments, the antagonist (e.g., an anti-PD-L1 antibody (e.g., atezolizumab) or an anti-PD-1 antibody) is administered to the subject intravenously at a dose of about 840 mg every 2 weeks, about 1200 mg every 3 weeks, or about 1680 mg of every 4 weeks. In some embodiments, the antagonist (e.g., an anti-PD-L1 antibody (e.g., atezolizumab) or an anti-PD-1 antibody) is administered at a dose of about 1200 mg of atezolizumab every three weeks. The dose may be administered as a single dose or as multiple doses (e.g., 2 or 3 doses), such as infusions. The dose of the antibody administered in a combination treatment may be reduced as compared to a single treatment. In some embodiments, the treatment regimen comprises administering intravenously to the subject about 1200 mg of atezolizumab every three weeks.

In some embodiments, the PD-1 axis binding antagonist (e.g., an anti-PD-L1 antibody (e.g., atezolizumab) or an anti-PD-1 antibody) and the IL6 antagonist (e.g., anti-IL6 receptor antibody, e.g. tocilizumab) are administered in a single dosing regimen. The administration of these agents may be concurrent or separate within the context of the dosing regimen.

The PD-1 axis binding antagonist (e.g., an anti-PD-L1 antibody (e.g., atezolizumab) or an anti-PD-1 antibody) and/or the IL6 antagonist (e.g., anti-IL6 receptor antibody, e.g. tocilizumab) may be administered in any suitable manner known in the art. For example, the PD-1 axis binding antagonist (e.g., an anti-PD-L1 antibody (e.g., atezolizumab) or an anti-PD-1 antibody) and the IL6 antagonist (e.g., anti-IL6 receptor antibody, e.g. tocilizumab) may be administered sequentially (at different times) or concurrently (at the same time). In some embodiments, the PD-1 axis binding antagonist is administered prior to the IL6 antagonist (e.g., anti-IL6 receptor antibody, e.g. tocilizumab). In other embodiments, the PD-1 axis binding antagonist is administered after the IL6 antagonist (e.g., anti-IL6 receptor antibody, e.g. tocilizumab). In yet other embodiments, the PD-1 axis binding antagonist is administered concurrently with the IL6 antagonist (e.g., anti-IL6 receptor antibody, e.g. tocilizumab). In some embodiments, the PD-1 axis binding antagonist (e.g., an anti-PD-L1 antibody (e.g., atezolizumab) or an anti-PD-1 antibody) is in a separate composition as the IL6 antagonist (e.g., anti-IL6 receptor antibody, e.g. tocilizumab). In some embodiments, the PD-1 axis binding antagonist (e.g., an anti-PD-L1 antibody (e.g., atezolizumab) or an anti-PD-1 antibody) is in the same composition as the IL6 antagonist (e.g., anti-IL6 receptor antibody, e.g. tocilizumab).

The PD-1 axis binding antagonist (e.g., an anti-PD-L1 antibody (e.g., atezolizumab) or an anti-PD-1 antibody) and the IL6 antagonist (e.g., anti-IL6 receptor antibody, e.g. tocilizumab) may be administered by the same route of administration or by different routes of administration. In some embodiments, the PD-1 axis binding antagonist (e.g., an anti-PD-L1 antibody (e.g., atezolizumab) or an anti-PD-1 antibody) and IL6 antagonist (e.g., anti-IL6 receptor antibody, e.g. tocilizumab) are administered intravenously, intramuscularly, subcutaneously, topically, orally, transdermally, intraperitoneally, intraorbitally, by implantation, by inhalation, intrathecally, intraventricularly, or intranasally. An effective amount of the PD-1 axis binding antagonist (e.g., an anti-PD-L1 antibody (e.g., atezolizumab) or an anti-PD-1 antibody) and IL6 antagonist (e.g., anti-IL6 receptor antibody, e.g. tocilizumab) may be administered for prevention or treatment of disease. The appropriate dosage of the PD-1 axis binding antagonist (e.g., an anti-PD-L1 antibody (e.g., atezolizumab) or an anti-PD-1 antibody) and/or the IL6 antagonist (e.g., anti-IL6 receptor antibody, e.g. tocilizumab) may be determined based on the type of disease to be treated, the type of the PD-1 axis binding antagonist and the IL6 antagonist (e.g., anti-IL6 receptor antibody, e.g. tocilizumab), the severity and course of the disease, the clinical condition of the individual, the individual's clinical history and response to the treatment, and the discretion of the attending physician. In some embodiments, the PD-1 axis binding antagonist (e.g., an anti-PD-L1 antibody (e.g., atezolizumab) or an anti-PD-1 antibody) and/or the IL6 antagonist (e.g., anti-IL6 receptor antibody, e.g. tocilizumab) is/are administered intravenously by infusion.

In some embodiments, the treatment may further comprise an additional therapy, such as chemotherapy and/or anti-angiogenic therapy (e.g. bevacizumab) discussed in more detail in Section VI. below.

Any suitable additional therapy known in the art or described herein may also be used. The additional therapy may be radiation therapy, surgery (e.g., transurethral bladder tumor resection (TURBT) or cystectomy (including a partial or radical cystectomy)), chemotherapy, gene therapy, DNA therapy, viral therapy, RNA therapy, immunotherapy, bone marrow transplantation, nanotherapy, monoclonal antibody therapy, ora combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, and the like). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PI3K/AKT/mTOR pathway, HSP90 inhibitor, tubulin inhibitor, apoptosis inhibitor, and/or chemopreventative agent. The additional therapy may be one or more of the chemotherapeutic agents described herein.

### IV. PD-1 Axis Binding Antagonists

In one embodiment, the methods herein involve treating cancer with a PD-1 axis binding antagonist.

For example, a PD-1 axis binding antagonist includes a PD-L1 binding antagonist, a PD-1 binding antagonist, and a PD-L2 binding antagonist. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No. Q9NZQ7.1. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some embodiments, PD-L1, PD-1, and PD-L2 are human PD-L1, PD-1, and PD-L2.

In some embodiments, the PD-1 axis binding antagonist is an anti-PD-L1 antibody. In some embodiments, the anti-PD-L1 antibody is atezolizumab, YW243.55.S70, MDX-1105, MEDI4736 (durvalumab), or MSB0010718C (avelumab). Antibody YW243.55.S70 is an anti-PD-L1 antibody described in WO 2010/077634. MDX-1105, also known as BMS-936559, is an anti-PD-L1 antibody described in WO2007/005874. MEDI4736 is an anti-PD-L1 monoclonal antibody described in WO2011/066389 and US2013/034559. In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab)₂ fragments. In some embodiments, the anti-PD-L1 antibody is a humanized antibody. In some embodiments, the anti-PD-L1 antibody is a human antibody.

Examples of anti-PD-L1 antibodies useful for the methods of this invention, and methods for making thereof are described in PCT Patent Application Nos. WO 2010/077634, WO 2007/005874, WO 2011/066389, and in US 2013/034559, which are incorporated herein by reference. The anti-PD-L1 antibodies useful in this invention, including compositions containing such antibodies, may be used as a monotherapy or in combination with one or more additional therapeutic agents, e.g., an IL6 antagonist.

In some embodiments, the PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen-binding fragment thereof, an immunoadhesin, a fusion protein, or oligopeptide.

Any suitable anti-PD-L1 antibody may be used in the methods and compositions provided herein. Anti-PD-L1 antibodies described in WO 2010/077634 A1 and U.S. Pat. No. 8,217,149 may be used in the methods and compositions provided herein. In some instances, the anti-PD-L1 antibody comprises a heavy chain variable region sequence of SEQ ID NO: 3 and/or a light chain variable region sequence of SEQ ID NO: 4. In a still further instance, provided is an isolated anti-PD-L1 antibody comprising a heavy chain variable region and/or a light chain variable region sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: EVQLVESGGGLVQPGGSLRLSCAASGFTFSDSWIHWVRQAPGKGLEWVAWISPYGGSTYYADSVKGRFTIS ADTSKNTAYLQMNSLRAEDTAVYYCARRHWPGGFDYWGQGTLVTVSS (SEQ ID NO: 3), and

(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In one instance, the anti-PD-L1 antibody comprises a heavy chain variable region comprising an HVR-H1, HVR-H2 and HVR-H3 sequence, wherein:

(SEQ ID NO: 5)
(a) the HVR-H1 sequence is GFTFSX₁SWIH;
(SEQ ID NO: 6)
(b) the HVR-H2 sequence is AWIX₂PYGGSX₃YYADSVKG;
(SEQ ID NO: 7)
(c) the HVR-H3 sequence is RHWPGGFDY;

further wherein: X₁ is D or G; X₂ is S or L; X₃ is T or S. In one specific aspect, X₁ is D; X₂ is S and X₃ is T. In another aspect, the polypeptide further comprises variable region heavy chain framework sequences juxtaposed between the HVRs according to the formula: (FR-H1)-(HVR-H1)-(FR-H2)-(HVR-H2)-(FR-H3)-(HVR-H3)-(FR-H4). In yet another aspect, the framework sequences are derived from human consensus framework sequences. In a further aspect, the framework sequences are VH subgroup III consensus framework. In a still further aspect, at least one of the framework sequences is the following:

(SEQ ID NO: 8)
FR-H1 is EVQLVESGGGLVQPGGSLRLSCAAS
(SEQ ID NO: 9)
FR-H2 is WVRQAPGKGLEWV
(SEQ ID NO: 10)
FR-H3 is RFTISADTSKNTAYLQMNSLRAEDTAVYYCAR
(SEQ ID NO: 11)
FR-H4 is WGQGTLVTVSS.

In a still further aspect, the heavy chain polypeptide is further combined with a variable region light chain comprising an HVR-L1, HVR-L2 and HVR-L3, wherein:

(SEQ ID NO: 12)
(a) the HVR-L1 sequence is RASQX₄X₅X₆TX₇X₈A;
(SEQ ID NO: 13)
(b) the HVR-L2 sequence is SASX₉LX₁₀S,;
(SEQ ID NO: 14)
(c) the HVR-L3 sequence is QQX₁₁X₁₂X₁₃X₁₄PX₁₅T;

wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, For W; X₁₃ is Y, N, A, T, G, F or I; X₁₄ is H, V, P, Tor I; Xis is A, W, R, P or T. Ina still further aspect, X₄ is D; X₅ is V; X₅ is S; X₇ is A; X₈ is V; X₉ is F; X₁₀ is Y; X₁₁ is Y; X₁₂ is L; X₁₃ is Y; X₁₄ is H; Xis is A.

In a still further aspect, the light chain further comprises variable region light chain framework sequences juxtaposed between the HVRs according to the formula: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the framework sequences are VL kappa I consensus framework. In a still further aspect, at least one of the framework sequence is the following:

(SEQ ID NO: 15)
FR-L1 is DIQMTQSPSSLSASVGDRVTITC
(SEQ ID NO: 16)
FR-L2 is WYQQKPGKAPKLLIY
(SEQ ID NO: 17)
FR-L3 is GVPSRFSGSGSGTDFTLTISSLQPEDFATYYC
(SEQ ID NO: 18)
FR-L4 is FGQGTKVEIKR.

In another instance, provided is an isolated anti-PD-L1 antibody or antigen binding fragment comprising a heavy chain and a light chain variable region sequence, wherein:

(a) the heavy chain comprises an HVR-H1, HVR-H2 and HVR-H3, wherein further:

(SEQ ID NO: 5)
(i) the HVR-H1 sequence is GFTFSX₁SWIH;
(SEQ ID NO: 6)
(ii) the HVR-H2 sequence is AWIX₂PYGGSX₃YYADSVKG
(SEQ ID NO: 7)
(iii) the HVR-H3 sequence is RHWPGGFDY_{,}
and

(b) the light chain comprises an HVR-L1, HVR-L2 and HVR-L3, wherein further:

(SEQ ID NO: 12)
(i) the HVR-L1 sequence is RASQX₄X₅X₆TX₇X₈A
(SEQ ID NO: 13)
(ii) the HVR-L2 sequence is SASX₉LX₁₀S;
and
(SEQ ID NO: 14)
(iii) the HVR-L3 sequence is QQX₁₁X₁₂X₁₃X₁₄PX₁₅T;

wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs:8, 9, 10, and 11. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, or
(b) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and 11. In a still further aspect, the light chain framework sequences are derived from a Kabat kappa I, II, II, or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

FR-H1
(SEQ ID NO: 27)
EVQLVESGGGLVQPGGSLRLSCAASGFTFS
FR-H2
(SEQ ID NO: 28)
WVRQAPGKGLEWVA
FR-H3
(SEQ ID NO: 10)
RFTISADTSKNTAYLQMNSLRAEDTAVYYCAR
FR-H4
(SEQ ID NO: 11)
WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

FR-L1
(SEQ ID NO: 15)
DIQMTQSPSSLSASVGDRVTITC
FR-L2
(SEQ ID NO: 16)
WYQQKPGKAPKLLIY
FR-L3
(SEQ ID NO: 17)
GVPSRFSGSGSGTDFTLTISSLQPEDFATYYC
FR-L4
(SEQ ID NO: 26)
FGQGTKVEIK.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(c) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, and/or
(d) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.
   In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and WGQGTLVTVSSASTK (SEQ ID NO: 29).

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18. In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In a still further instance, provided is an isolated anti-PD-L1 antibody comprising a heavy chain and a light chain variable region sequence, wherein:

(a) the heavy chain sequence has at least 85% sequence identity to the heavy chain sequence: EVQLVESGGGLVQPGGSLRLSCAASGFTFSDSWIHWVRQAPGKGLEWVAWISPYGGSTYYADSVKGRFTIS ADTSKNTAYLQMNSLRAEDTAVYYCARRHWPGGFDYWGQGTLVTVSSASTK (SEQ ID NO: 25), or

(b) the light chain sequences has at least 85% sequence identity to the light chain sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4. In some instances, provided is an isolated anti-PD-L1 antibody comprising a heavy chain and a light chain variable region sequence, wherein the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 25. In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4 and the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 25. In some instances, one, two, three, four, or five amino acid residues at the N-terminal of the heavy and/or light chain may be deleted, substituted or modified.

In a still further instance, provided is an isolated anti-PD-L1 antibody comprising a heavy chain and a light chain sequence, wherein:

(a) the heavy chain sequence has at least 85% sequence identity to the heavy chain sequence: EVQLVESGGGLVQPGGSLRLSCAASGFTFSDSWIHWVRQAPGKGLEWVAWISPYGGSTYYADSVKGRFTIS ADTSKNTAYLQMNSLRAEDTAVYYCARRHWPGGFDYWGQGTLVTVSSASTKGPSVFPLAPSSKSTSGGTA ALGCLVKDYFPEPVTVSWNSGALTSGVHTFPAVLQSSGLYSLSSVVTVPSSSLGTQTYICNVNHKPSNTKVD KKVEPKSCDKTHTCPPCPAPELLGGPSVFLFPPKPKDTLMISRTPEVTCWVDVSHEDPEVKFNVVYVDGVEV HNAKTKPREEQYASTYRVVSVLTVLHQDWLNGKEYKCKVSNKALPAPI EKTISKAKGQPREPQVYTLPPSRE EMTKNQVSLTCLVKGFYPSDIAVEWESNGQPENNYKTTPPVLDSDGSFFLYSKLTVDKSRWQQGNVFSCSV MHEALHNHYTQKSLSLSPG (SEQ ID NO: 30), and/or

(b) the light chain sequences has at least 85% sequence identity to the light chain sequence:

(SEQ ID NO: 31)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31. In some instances, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30. In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30. In some instances, provided is an isolated anti-PD-L1 antibody comprising a heavy chain comprising the amino acid sequence of SEQ ID NO:30 and a light chain sequence comprising the amino acid sequence of SEQ ID NO:31.

In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the instances herein, the isolated anti-PD-L1 antibody can bind to a human PD-L1, for example a human PD-L1 as shown in UniProtKB/Swiss-Prot Accession No. Q9NZQ7.1, or a variant thereof.

In a still further instance, provided is an isolated nucleic acid encoding any of the antibodies described herein. In some instances, the nucleic acid further comprises a vector suitable for expression of the nucleic acid encoding any of the previously described anti-PD-L1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen binding fragment thereof, may be made using methods known in the art, for example, by a process comprising culturing a host cell containing nucleic acid encoding any of the previously described anti-PD-L1 antibodies or antigen-binding fragments in a form suitable for expression, under conditions suitable to produce such antibody or fragment, and recovering the antibody or fragment.

In some embodiments, the PD-1 axis binding antagonist is a PD-1 binding antagonist. In some embodiments, the PD-1 binding antagonist is an anti-PD-1 antibody (e.g., a human antibody, a humanized antibody, or a chimeric antibody). Any suitable anti-PD-1 antibody may be used in the context of the invention. In some embodiments, the anti-PD-1 antibody is selected from the group consisting of MDX-1106 (nivolumab), MK-3475 (pembrolizumab), MEDI-0680 (AMP-514), PDR001, REGN2810, and BGB-108. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. In some embodiments, the PD-L1 binding antagonist is anti-PD-L1 antibody. MDX-1106, also known as MDX-1106-04, ONO-4538, BMS-936558, or nivolumab, is an anti-PD-1 antibody described in WO2006/121168. MK-3475, also known as lambrolizumab, is an anti-PD-1 antibody described in WO2009/114335. AMP-224, also known as B7-DCIg, is a PD-L2-Fc fusion soluble receptor described in WO2010/027827 and WO2011/066342. In some instances, the anti-PD-1 antibody is MDX-1106. Alternative names for “MDX-1106” include MDX-1106-04, ONO-4538, BMS-936558, and nivolumab. In some instances, the anti-PD-1 antibody is nivolumab (CAS Registry Number: 946414-94-4). In a still further instance, provided is an isolated anti-PD-1 antibody comprising a heavy chain variable region comprising the heavy chain variable region amino acid sequence from SEQ ID NO: 1 and/or a light chain variable region comprising the light chain variable region amino acid sequence from SEQ ID NO: 2.

In a still further instance, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: QVQLVESGGGVVQPGRSLRLDCKASGITFSNSGMHWVRQAPGKGLEWVAVIWYDGSKRYYADSVKGRFTI SRDNSKNTLFLQMNSLRAEDTAVYYCATNDDYWGQGTLVTVSSASTKGPSVFPLAPCSRSTSESTAALGCLV KDYFPEPVTVSWNSGALTSGVHTFPAVLQSSGLYSLSSWTVPSSSLGTKTYTCNVDHKPSNTKVDKRVESK YGPPCPPCPAPEFLGGPSVFLFPPKPKDTLMISRTPEVTCWVDVSQEDPEVQFNWYVDGVEVHNAKTKPR EEQFNSTYRVVSVLTVLHQDWLNGKEYKCKVSNKGLPSSIEKTISKAKGQPREPQVYTLPPSQEEMTKNQVS LTCLVKGFYPSDIAVEWESNGQPENNYKTTPPVLDSDGSFFLYSRLTVDKSRWQEGNVFSCSVMHEALHNH YTQKSLSLSLGK (SEQ ID NO: 1), and

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In a still further embodiment, provided is an isolated nucleic acid encoding any of the antibodies described herein. In some embodiments, the nucleic acid further comprises a vector suitable for expression of the nucleic acid encoding any of the previously described anti-PD-1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen-binding fragment thereof, may be made using methods known in the art, for example, by a process comprising culturing a host cell containing nucleic acid encoding any of the previously described anti-PD-1 antibodies in a form suitable for expression, under conditions suitable to produce such antibody or fragment, and recovering the antibody or fragment, or according to any method described below.

### V. IL6 Antagonists

IL6 antagonists contemplated herein include antagonists that bind to IL6 or IL6 receptor.

In one embodiment, the IL6 antagonist is an antibody.

In one embodiment, the IL6 antagonist is an antibody that binds IL6 receptor. Antibodies that bind IL-6R include tocilizumab (including intravenous, iv, and subcutaneous sc formulations thereof) (Chugai, Roche, Genentech), satralizumab (Chugai, Roche, Genentech), sarilumab (Sanofi, Regeneron), NI-1201 (Novimmune and Tiziana), and vobarilizumab (Ablynx).

In one embodiment, the IL6 antagonist is tocilizumab.

Tocilizumab, also named Myeloma Receptor Antibody (MRA), is a recombinant humanized monoclonal antibody that selectively binds to human interleukin-6 receptor (IL-6R). It is an IgG1 K (gamma 1, kappa) antibody with a typical H2L2 structure. The tocilizumab molecule is composed of two heterodimers. Each of the heterodimers is composed of a heavy (H) and a light (L) polypeptide chain. The four polypeptide chains are linked intra- and inter-molecularly by disulfide linkages. The molecular formula and theoretical molecular weight of the tocilizumab antibody are as follows:

Molecular formula: C₆₄₂₈H₉₉₇₆N₁₇₂₀O₂₀₁₈S₄₂ (polypeptide moiety only)

Molecular weight: 144,985 Da (polypeptide moiety only).

The amino acid sequence of the light chain deduced from complimentary deoxyribonucleic acid (cDNA) sequences and confirmed by liquid chromatography mass-spectrometry (LC-MS) peptide mapping is in SEQ ID Nos. 32 and 33. The five light chain cysteine residues of each heterodimer are involved in two intrachain disulfide linkages and one interchain disulfide linkage:

Intrachain linkages: Cys_{L23}-Cys_{L88} and Cys_{L134}-Cys_{L194}

Linkage between heavy and light chain: Cys_{L214} and Cys_{H222}

Assignments of the disulfide linkages are based on sequence homology to other IgG1 antibodies and were confirmed by liquid chromatography mass-spectrometry (LC-MS) peptide mapping performed using material from the fourth generation (G4) process. CysL, and CysH,denote cysteine residues at position x of the light and heavy chains, respectively.

| SEQ ID NO. 32 | | |
|---|---|---|
| Amino Acid Sequence of the L Chain of the Tocilizumab Molecule | | |
| 1 | DIQMTQSPSS LSASVGDRVT ITCRASQDIS SYLNWYQQKP GKAPKLLIYY | 50 |
| 51 | TSRLHSGVPS RFSGSGSGTD FTFTISSLQP EDIATYYCQQ GNTLPYTFGQ | 100 |
| 101 | GTKVEIKRTV AAPSVFIFPP SDEQLKSGTA SVVCLLNNFY PREAKVQWKV | 150 |
| 151 | DNALQSGNSQ ESVTEQDSKD STYSLSSTLT LSKADYEKHK VYACEVTHQG | 200 |
| 201 | LSSPVTKSFN RGEC | 214 |

Note:
The entire sequence has been determined by LC-MS peptide mapping.

The eleven heavy chain cysteine residues of each heterodimer are involved in four intrachain disulfide linkages, two interchain disulfide linkages between the two heavy chains and the third interchain disulfide linkage between the heavy chain and the light chain of each of the heterodimers:
Intrachain linkages: Cys_{H22}-Cys_{H96}, Cys_{H146}-Cys_{H202}, Cys_{H263}-Cys_{H323} and Cys_{H369}-Cys_{H427}
Linkages between the two heavy chains: Cys_{H228}-Cys_{H228} and Cys_{H231}-Cys_{H231}
Linkage between heavy and light chain: Cys_{L214}-Cys_{H222}

Assignments of the disulfide linkages are based on sequence homology to other IgG1 antibodies and were confirmed by LC-MS peptide mapping performed using material from the G4 process.

### SEQ ID NO. 33 Amino Acid Sequence of the H Chain of the Tocilizumab Molecule

| | | |
|---|---|---|
| 1 | pEVQLQESGPG LVRPSQTLSL TCTVSGYSIT SDRAWSWVRQ PPGRGLEWIG | 50 |
| 51 | YISYSGITTY NPSLKSRVTM LRDTSKNQFS LRLSSVTAAD TAVYYCARSL | 100 |
| 101 | ARTTAMDYWG QGSLVTVSSA STKGPSVFPL APSSKSTSGG TAALGCLVKD | 150 |
| 151 | YFPEPVTVSW NSGALTSGVH TFPAVLQSSG LYSLSSVVTV PSSSLGTQTY | 200 |
| 201 | ICNVNHKPSN TKVDKKVEPK SCDKTHTCPP CPAPELLGGP SVFLEPPKPK | 250 |
| 251 | DTLMISRTPE VTCVVVDVSH EDPEVKFNWY VDGVEVHNAK TKPREEQYNS | 300 |
| 301 | TYRVVSVLTV LHQDWLNGKE YKCKVSNKAL PAPIEKTISK AKGQPREPQV | 350 |
| 351 | YTLPPSRDEL TKNQVSLTCL VKGPYPSDIA VEWESNGQPE NNYKTTPPVL | 400 |
| 401 | DSDGSFFLYS KLTVDKSRWQ QGNVPSCSVM HEALENHYTQ KSLSLSPG | 448 |

Note:
The entire sequence has been determined by LC-MS peptide mapping. The N-terminus of the heavy chain has been determined to be predominantly a pyroglutamic acid residue (pE).

In one embodiment, the IL6 antagonist is satralizumab. Satralizumab (also called SA237) is a humanized monoclonal antibody that binds IL6 receptor. See U.S. Pat. No. 8,562,991.

In one embodiment, the IL6 antagonist is a monoclonal antibody that binds IL6.

Antibodies that bind IL-6 include sirukumab (Centecor, Janssen), olokizumab (UCB), clazakizumab (BMS and Alder), siltuximab (Janssen), EBI-031 (Eleven Biotherapeutics and Roche).

In one embodiment, the IL6 antagonist is olamkicept. Olamkicept is a recombinant protein that fuses the extracellular domain of the signal transducing subunit of the IL-6 receptor, IL-6Rβ (glycoprotein 130, gp130), to a human IgG Fc fragment. The full construct is a dimer of covalently linked identical peptide chains. Mechanistically olamkicept acts as an inhibitor of the IL-6 signalling pathway. Olamkicept inhibits trans signalling by the soluble IL-6 receptor (sIL-6R).

### VI. Further Combination Therapies

Besides the PD-1 axis binding antagonist and IL6 antagonist, the present invention contemplates additional drugs to be combined therewith. For example chemotherapeutic agent(s) and/or anti-antiogenic agents.

For example, for therapy of breast cancer, such as TNBC, chemotherapeutic agents that can be further combined include taxoids (such as paclitaxel and docetaxel and modified forms thereof such as nanoparticle albumin-bound paclitaxel (“Nab-paclitaxel”). Other chemotherapies for breast cancer include: anthracyclines, carboplatin, gemcitabine, capecitabine, vinorelbine, eribulin, and ixabepilone For urothelial or bladder cancer, chemotherapeutic agents that can be further combined include platinum-containing chemotherapy, e.g. cisplatin, and the combination of gemcitabine and cisplatin (GC).

For NSCLC, further drugs to combine with the combination include: bevacizumab, paclitaxel, and/or carboplatin; paclitaxel (e.g. Nab-paclitaxel) and/or carboplatin.

For Small Cell Lung Cancer (SCLC), further chemotherapeutic agents include carboplatin and/or etoposide.

For liver cancer, including hepatocellular carcinoma (HCC), another drug to combine with the PD-1 axis binding antagonist (e.g. atezolizumab) and IL6 antagonist (e.g. tocilizumab) comprises a VEGF antagonist (e.g. an anti-VEGF antibody, such as bevacizumab). Exemplary dosages for bevacizumab include 5, 7.5, 10, or 15 mg/kg, e.g. administered every 2 weeks or every 3 weeks. In one embodiment, bevacizumab is administered at a dose of 15 mg/kg very 3 weeks.

Also provided herein are methods for treating cancer in a subject comprising administering to the subject a treatment regimen comprising an effective amount of a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) in conjunction with another anti-cancer agent or cancer therapy. In some embodiments, the methods comprise administering to the individual a PD-1 axis binding antagonist (e.g., an anti-PD-L1 antibody (e.g., atezolizumab) or an anti-PD-1 antibody), an IL6 antagonist (e.g. an anti-IL6 receptor antibody (e.g. tocilizumab)), and an additional therapeutic agent.

In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an additional chemotherapy or chemotherapeutic agent. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with a radiation therapy or radiotherapeutic agent. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with a targeted therapy or targeted therapeutic agent. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an immunotherapy or immunotherapeutic agent, for example, a monoclonal antibody.

Without wishing to be bound to theory, it is thought that enhancing T cell stimulation, by promoting an activating co-stimulatory molecule or by inhibiting a negative co-stimulatory molecule, may promote tumor cell death, thereby treating or delaying progression of cancer. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an agonist directed against an activating co-stimulatory molecule. In some embodiments, an activating co-stimulatory molecule may include CD40, CD226, CD28, OX40, GITR, CD137, CD27, HVEM, or CD127. In some embodiments, the agonist directed against an activating co-stimulatory molecule is an agonist antibody that binds to CD40, CD226, CD28, OX40, GITR, CD137, CD27, HVEM, or CD127. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an antagonist directed against an inhibitory co-stimulatory molecule. In some embodiments, an inhibitory co-stimulatory molecule may include CTLA-4 (also known as CD152), PD-1, TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, PD-1, TIM-3, BTLA, VISTA, LAG-3, B7-H3, B7-H4, IDO, TIGIT, MICA/B, or arginase.

In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an antagonist directed against CTLA-4 (also known as CD152), for example, a blocking antibody. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with ipilimumab (also known as MDX-010, MDX-101, or YERVOY®). In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with tremelimumab (also known as ticilimumab or CP-675,206). In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an antagonist directed against B7-H3 (also known as CD276), for example, a blocking antibody. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with MGA271. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with an antagonist directed against a TGF beta, for example, metelimumab (also known as CAT-192), fresolimumab (also known as GC1008), or LY2157299.

In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with a treatment comprising adoptive transfer of a T cell (e.g., a cytotoxic T cell or CTL) expressing a chimeric antigen receptor (CAR). In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with a treatment comprising adoptive transfer of a T cell comprising a dominant-negative TGF beta receptor, e.g., a dominant-negative TGF beta type II receptor. In some embodiments, a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) may be administered in conjunction with a treatment comprising a HERCREEM protocol (see, e.g., ClinicalTrials.gov Identifier NCT00889954).

In some embodiments, a PD-1 axis binding antagonist and/or an IL6 antagonist may be administered in conjunction with an agonist directed against CD137 (also known as TNFRSF9, 4-1BB, or ILA), for example, an activating antibody. In some embodiments, a PD-1 axis binding antagonist and/or an IL6 antagonist may be administered in conjunction with urelumab (also known as BMS-663513). In some embodiments, a PD-1 axis binding antagonist and/or an IL6 antagonist may be administered in conjunction with an agonist directed against CD40, for example, an activating antibody. In some embodiments, a PD-1 axis binding antagonist and/or an IL6 antagonist may be administered in conjunction with CP-870893. In some embodiments, a PD-1 axis binding antagonist and/or an IL6 antagonist may be administered in conjunction with an agonist directed against OX40 (also known as CD134), for example, an activating antibody. In some embodiments, a PD-1 axis binding antagonist and/or an IL6 antagonist may be administered in conjunction with an anti-OX40 antibody (e.g., AgonOX). In some embodiments, a PD-1 axis binding antagonist and/or an IL6 antagonist may be administered in conjunction with an agonist directed against CD27, for example, an activating antibody. In some embodiments, a PD-1 axis binding antagonist and/or an IL6 antagonist may be administered in conjunction with CDX-1127. In some embodiments, a PD-1 axis binding antagonist and/or an IL6 antagonist may be administered in conjunction with an antagonist directed against indoleamine-2,3-dioxygenase (IDO). In some embodiments, the IDO antagonist is 1-methyl-D-tryptophan (also known as 1-D-MT).

In some embodiments, a PD-1 axis binding antagonist and/or an IL6 antagonist may be administered in conjunction with an antibody-drug conjugate. In some embodiments, the antibody-drug conjugate comprises mertansine or monomethyl auristatin E (MMAE). In some embodiments, a PD-1 axis binding antagonist and/or an IL6 antagonist may be administered in conjunction with and anti-NaPi2b antibody-MMAE conjugate (also known as DNIB0600A or RG7599). In some embodiments, a PD-1 axis binding antagonist and/or an IL6 antagonist may be administered in conjunction with trastuzumab emtansine (also known as T-DM1, ado-trastuzumab emtansine, or KADCYLA®, Genentech). In some embodiments, a PD-1 axis binding antagonist and/or an IL6 antagonist may be administered in conjunction with DMUC5754A. In some embodiments, a PD-1 axis binding antagonist and/or an IL6 antagonist may be administered in conjunction with an antibody-drug conjugate targeting the endothelin B receptor (EDNBR), for example, an antibody directed against EDNBR conjugated with MMAE.

In some embodiments, a PD-1 axis binding antagonist and/or an IL6 antagonist may be administered in conjunction with an angiogenesis inhibitor. In some embodiments, a PD-1 axis binding antagonist and/or an IL6 antagonist may be administered in conjunction with an antibody directed against angiopoietin 2 (also known as Ang2). In some embodiments, a PD-1 axis binding antagonist and/or an IL6 antagonist may be administered in conjunction with MEDI3617.

In some embodiments, a PD-1 axis binding antagonist and/or an IL6 antagonist may be administered in conjunction with an antineoplastic agent. In some embodiments, a PD-1 axis binding antagonist and/or an IL6 antagonist may be administered in conjunction with an agent targeting CSF-1R (also known as M-CSFR or CD115). In some embodiments, a PD-1 axis binding antagonist and/or an IL6 antagonist may be administered in conjunction with anti-CSF-1R (also known as IMC-CS4). In some embodiments, a PD-1 axis binding antagonist and/or an IL6 antagonist may be administered in conjunction with an interferon, for example interferon alpha or interferon gamma. In some embodiments, a PD-1 axis binding antagonist and/or an IL6 antagonist may be administered in conjunction with Roferon-A (also known as recombinant Interferon alpha-2a). In some embodiments, a PD-1 axis binding antagonist and/or an IL6 antagonist may be administered in conjunction with GM-CSF (also known as recombinant human granulocyte macrophage colony stimulating factor, rhu GM-CSF, sargramostim, or LEUKINE®). In some embodiments, a PD-1 axis binding antagonist and/or an IL6 antagonist may be administered in conjunction with IL-2 (also known as aldesleukin or PROLEUKIN®). In some embodiments, a PD-1 axis binding antagonist and/or an IL6 antagonist may be administered in conjunction with IL-12. In some embodiments, a PD-1 axis binding antagonist and/or an IL6 antagonist may be administered in conjunction with an antibody targeting CD20. In some embodiments, the antibody targeting CD20 is obinutuzumab (also known as GA101 or GAZYVA®) or rituximab. In some embodiments, a PD-1 axis binding antagonist and/or an IL6 antagonist may be administered in conjunction with an antibody targeting GITR. In some embodiments, the antibody targeting GITR is TRX518.

In some embodiments, a PD-1 axis binding antagonist and/or an IL6 antagonist may be administered in conjunction with a cancer vaccine. In some embodiments, the cancer vaccine is a peptide cancer vaccine, which in some embodiments is a personalized peptide vaccine. In some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21 (2013)). In some embodiments, a PD-1 axis binding antagonist and/or an IL6 antagonist may be administered in conjunction with an adjuvant. In some embodiments, a PD-1 axis binding antagonist and/or an IL6 antagonist may be administered in conjunction with a treatment comprising a TLR agonist, for example, Poly-ICLC (also known as HILTONOL®), LPS, MPL, or CpG ODN. In some embodiments, a PD-1 axis binding antagonist and/or an IL6 antagonist may be administered in conjunction with tumor necrosis factor (TNF) alpha. In some embodiments, a PD-1 axis binding antagonist and/or an IL6 antagonist may be administered in conjunction with IL-1. In some embodiments, a PD-1 axis binding antagonist and/or an IL6 antagonist may be administered in conjunction with HMGB1. In some embodiments, a PD-1 axis binding antagonist and/or an IL6 antagonist may be administered in conjunction with an IL-10 antagonist. In some embodiments, a PD-1 axis binding antagonist and/or an IL6 antagonist may be administered in conjunction with an IL-4 antagonist. In some embodiments, a PD-1 axis binding antagonist and/or an IL6 antagonist may be administered in conjunction with an IL-13 antagonist. In some embodiments, a PD-1 axis binding antagonist and/or an IL6 antagonist may be administered in conjunction with an HVEM antagonist. In some embodiments, a PD-1 axis binding antagonist and/or an IL6 antagonist may be administered in conjunction with an ICOS agonist, e.g., by administration of ICOS-L, or an agonistic antibody directed against ICOS. In some embodiments, a PD-1 axis binding antagonist and/or an IL6 antagonist may be administered in conjunction with a treatment targeting CX3CL1. In some embodiments, a PD-1 axis binding antagonist and/or an IL6 antagonist may be administered in conjunction with a treatment targeting CXCL9. In some embodiments, a PD-1 axis binding antagonist and/or an IL6 antagonist may be administered in conjunction with a treatment targeting CXCL10. In some embodiments, a PD-1 axis binding antagonist and/or an IL6 antagonist may be administered in conjunction with a treatment targeting CCL5. In some embodiments, a PD-1 axis binding antagonist and/or an IL6 antagonist may be administered in conjunction with an LFA-1 or ICAM1 agonist. In some embodiments, a PD-1 axis binding antagonist and/or an IL6 antagonist may be administered in conjunction with a Selectin agonist.

In some embodiments, a PD-1 axis binding antagonist and/or an IL6 antagonist may be administered in conjunction with a targeted therapy. In some embodiments, a PD-1 axis binding antagonist and/or an IL6 antagonist may be administered in conjunction with an inhibitor of B-Raf. In some embodiments, a PD-1 axis binding antagonist and/or an IL6 antagonist may be administered in conjunction with vemurafenib (also known as ZELBORAF®). In some embodiments, a PD-1 axis binding antagonist and/or an IL6 antagonist may be administered in conjunction with dabrafenib (also known as TAFINLAR®). In some embodiments, a PD-1 axis binding antagonist and/or an IL6 antagonist may be administered in conjunction with erlotinib (also known as TARCEVA®). In some embodiments, a PD-1 axis binding antagonist and/or an IL6 antagonist may be administered in conjunction with an inhibitor of a MEK, such as MEK1 (also known as MAP2K1) or MEK2 (also known as MAP2K2). In some embodiments, a PD-1 axis binding antagonist and/or an IL6 antagonist may be administered in conjunction with cobimetinib (also known as GDC-0973 or XL-518). In some embodiments, a PD-1 axis binding antagonist and/or an IL6 antagonist may be administered in conjunction with trametinib (also known as MEKINIST®). In some embodiments, a PD-1 axis binding antagonist and/or an IL6 antagonist may be administered in conjunction with an inhibitor of K-Ras. In some embodiments, a PD-1 axis binding antagonist and/or an IL6 antagonist may be administered in conjunction with an inhibitor of c-Met. In some embodiments, a PD-1 axis binding antagonist and/or an IL6 antagonist may be administered in conjunction with onartuzumab (also known as MetMAb). In some embodiments, a PD-1 axis binding antagonist and/or an IL6 antagonist may be administered in conjunction with an inhibitor of Alk. In some embodiments, a PD-1 axis binding antagonist and/or an IL6 antagonist may be administered in conjunction with AF802 (also known as CH5424802 or alectinib). In some embodiments, a PD-1 axis binding antagonist and/or an IL6 antagonist may be administered in conjunction with an inhibitor of a phosphatidylinositol 3-kinase (PI3K). In some embodiments, a PD-1 axis binding antagonist and/or an IL6 antagonist may be administered in conjunction with BKM120. In some embodiments, a PD-1 axis binding antagonist and/or an IL6 antagonist may be administered in conjunction with idelalisib (also known as GS-1101 or CAL-101). In some embodiments, a PD-1 axis binding antagonist and/or an IL6 antagonist may be administered in conjunction with perifosine (also known as KRX-0401). In some embodiments, a PD-1 axis binding antagonist and/or an IL6 antagonist may be administered in conjunction with an inhibitor of an Akt. In some embodiments, a PD-1 axis binding antagonist and/or an IL6 antagonist may be administered in conjunction with MK2206. In some embodiments, a PD-1 axis binding antagonist and/or an IL6 antagonist may be administered in conjunction with GSK690693. In some embodiments, a PD-1 axis binding antagonist and/or an IL6 antagonist may be administered in conjunction with GDC-0941. In some embodiments, a PD-1 axis binding antagonist and/or an IL6 antagonist may be administered in conjunction with an inhibitor of mTOR. In some embodiments, a PD-1 axis binding antagonist and/or an IL6 antagonist may be administered in conjunction with sirolimus (also known as rapamycin). In some embodiments, a PD-1 axis binding antagonist and/or an IL6 antagonist may be administered in conjunction with temsirolimus (also known as CCI-779 or TORISEL®). In some embodiments, a PD-1 axis binding antagonist and/or an IL6 antagonist may be administered in conjunction with everolimus (also known as RAD001). In some embodiments, a PD-1 axis binding antagonist and/or an IL6 antagonist may be administered in conjunction with ridaforolimus (also known as AP-23573, MK-8669, or deforolimus). In some embodiments, a PD-1 axis binding antagonist and/or an IL6 antagonist may be administered in conjunction with OSI-027. In some embodiments, a PD-1 axis binding antagonist and/or an IL6 antagonist may be administered in conjunction with AZD8055. In some embodiments, a PD-1 axis binding antagonist and/or an IL6 antagonist may be administered in conjunction with INK128. In some embodiments, a PD-1 axis binding antagonist and/or an IL6 antagonist may be administered in conjunction with a dual PI3K/mTOR inhibitor. In some embodiments, a PD-1 axis binding antagonist and/or an IL6 antagonist may be administered in conjunction with XL765. In some embodiments, a PD-1 axis binding antagonist and/or an IL6 antagonist may be administered in conjunction with GDC-0980. In some embodiments, a PD-1 axis binding antagonist and/or an IL6 antagonist may be administered in conjunction with BEZ235 (also known as NVP-BEZ235). In some embodiments, a PD-1 axis binding antagonist and/or an IL6 antagonist may be administered in conjunction with BGT226. In some embodiments, a PD-1 axis binding antagonist and/or an IL6 antagonist may be administered in conjunction with GSK2126458. In some embodiments, a PD-1 axis binding antagonist and/or an IL6 antagonist may be administered in conjunction with PF-04691502. In some embodiments, a PD-1 axis binding antagonist and/or an IL6 antagonist may be administered in conjunction with PF-05212384 (also known as PKI-587).

In any of the preceding embodiments, the PD-1 axis binding antagonist may be a human PD-1 axis binding antagonist.

In any of the preceding embodiments, the PD-1 axis binding antagonist is an anti-PD-L1 antibody (e.g., atezolizumab) or an anti-PD-1 antibody.

In any of the preceding embodiments, the platinum-based chemotherapy includes a platinum-based chemotherapeutic agent (e.g., cisplatin or carboplatin). In some embodiments, the platinum-based chemotherapy includes cisplatin. In some embodiments, the platinum-based chemotherapy includes carboplatin. In some embodiments, the platinum-based chemotherapy further includes one or more additional chemotherapeutic agents, e.g., a nucleoside analog. In some embodiments, the nucleoside analog is gemcitabine. In some embodiments, the platinum-based chemotherapy includes cisplatin and gemcitabine.

In other embodiments, the platinum-based chemotherapy includes carboplatin and gemcitabine.

### VII. PD-L1, IL6, and CRP Biomarker Assessment

Optionally, the patient treated herein has been subjected to an assay which has found the patient or his or her tumor to have one or more of the following biomarker measurements:
1. C-reactive protein (CRP) level above the upper limit of normal;
2. ≥3 mg/L CRP;
3. ≥10 mg/L CRP;
4. ≥3 mg/L CRP as measured by enzyme-linked immunosorbent assay (ELISA);
5. ≥10 mg/L CRP as measured by enzyme-linked irnmunosorbent assay (ELISA);
6. IL-6 level above the upper limit of normal;
7. ≥10 pg/mL IL-6;
8. ≥15 pg/mL IL-6;
9. ≥10 pg/mL IL-6 as measured by enzyme-linked immunosorbent assay (ELISA);
10. ≥15 pg/mL IL-6 as measured by enzyme-linked immunosorbent assay (ELISA);
11. PD-L1 expression (“PD-L1 positive”);
12. PD-L1 stained tumor cells (TC) or tumor-infiltrating immune cells (IC);
13. PD-L1 stained tumor cells (TC) or tumor-infiltrating immune cells (IC) covering 1% of the tumor area;
14. PD-L1 stained tumor cells (TC) or tumor-infiltrating immune cells (IC) covering 1% of the tumor area;
15. PD-L1 expression as determined by PD-L1 IHC 22C3 pharmDx (Merck);
16. PD-L1 expression as determined by PD-L1 (SP142) Assay (Ventana); and/or
17. PD-L1 expression as determined by PD-L1 (SP263) Assay (Ventana).

In one embodiment, the patient has C-reactive protein (CRP) level above the upper limit of normal.

In one embodiment, the patient has IL-6 level above the upper limit of normal.

In one embodiment, the patient's cancer expresses PD-L1.

In one embodiment, the patient has C-reactive protein (CRP) level above the upper limit of normal and expresses PD-L1.

In one embodiment, the patient has C-reactive protein (CRP) and IL-6 levels above the upper limit of normal.

In one embodiment, the patient has C-reactive protein (CRP) and IL-6 levels above the upper limit of normal and expresses PD-L1.

In one embodiment, the patient has IL-6 levels above the upper limit of normal and expresses PD-L1.

In one embodiment, the assay (measuring CRP and/or IL-6 and/or PD-L1) is performed on a sample from the patient obtained from the patient prior to administration of an anti-cancer therapy.

In one embodiment, the assay (measuring CRP and/or IL-6 and/or PD-L1) is performed on a sample from the patient obtained from the patient after administration of an anti-cancer therapy, including after administration of the PD-1 axis binding antagonist and IL-6 antagonist.

In one embodiment, the sample is a blood sample from the patient.

In one embodiment, the sample from the patient is a whole blood sample, a plasma sample, a serum sample, or a combination thereof.

In one embodiment, the sample is an archival sample, a fresh sample, or a frozen sample.

In one embodiment, the sample from the patient is a tumor tissue sample, e.g. a formalin-fixed and paraffin-embedded (FFPE) sample, an archival sample, a fresh sample, or a frozen sample.

In one embodiment, the expression level of IL-6 in a sample from the individual has been determined to be above a reference IL-6 expression level, e.g. wherein the reference IL-6 expression level is a pre-assigned IL-6 expression level. For example, the expression level of IL-6 in the sample is an expression level of IL-6 that is at least four standard deviations above the reference IL-6 expression level.

In one embodiment, the expression level of IL-6 in the sample is a protein expression level of IL-6.

In one embodiment, the expression level of IL-6 is an mRNA expression level of IL-6. Assays for measuring mRNA expression level of IL-6 include in situ hybridization (ISH) (e.g. using a probe targeting nucleotides 2-1082 of an IL-6 mRNA), RNA-seq, RT-qPCR, qPCR, multiplex qPCR or RT-qPCR, microarray analysis, SAGE, MassARRAY technique, FISH, or a combination thereof.

In one embodiment, the reference IL-6 expression level is between about 10 pg/mL to about 15 pg/mL.

In one embodiment, the reference IL-6 expression level is 10 pg/mL.

In one embodiment, the reference IL-6 expression level is an expression level of IL-6 in a reference population of healthy individuals.

In one embodiment, the reference IL-6 expression level is an expression level of IL-6 in a reference population of individuals with the tumor type being treated.

CRP tests that measure markedly high levels of the CRP protein are available in the art. Such tests can measure CRP in the range from 10 to 1000 mg/L.

In one embodiment, the CRP assay is a highly sensitive CRP (hsCRP) assay.

In one embodiment, the CRP assay is an ELISA assay.

In one embodiment, the CRP assay is a Luminex CRP assay.

Normal CRP levels are below 3.0 mg/L, Levels of CRP >3.0 mg./L can put a patient at a higher than average risk for heart disease. Levels of CRP >10.0 mg./L signify infection or an inflammatory condition.

In one embodiment, the expression level of CRP in a sample from the patient has been determined to be above a reference CRP expression level, e.g. 3 mg/L or 10 mg/L.

In one embodiment, the reference CRP expression level is a pre-assigned CRP expression level.

In one embodiment, the expression level of CRP in the sample is a protein expression level of CRP or an mRNA expression level of CRP.

In one embodiment, the protein expression level of CRP is measured using nephelometry.

In one embodiment, the reference CRP expression level is an expression level of CRP in a reference population of healthy individuals.

In one embodiment, the reference CRP expression level is an expression level of CRP in a reference population of individuals with the tumor type being treated.

The expression of PD-L1 may be assessed in a subject treated according to any of the methods and compositions for use described herein. In some embodiments, the method includes determining the expression level of PD-L1 in a biological sample (e.g., a tumor sample) obtained from the subject. In other embodiments, the expression level of PD-L1 in a biological sample (e.g., a tumor sample) obtained from the subject has been determined prior to initiation of treatment. In yet other embodiments, the expression level of PD-L1 in a biological sample (e.g., a tumor sample) obtained from the subject may be determined after initiation of treatment.

In some embodiments, a tumor sample obtained from the subject has been determined to have a detectable expression level of PD-L1 in tumor-infiltrating immune cells that comprise about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor sample. For example, in some embodiments, a tumor sample obtained from the subject has been determined to have a detectable expression level of PD-L1 in tumor-infiltrating immune cells that comprise from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor sample.

In some embodiments, a tumor sample obtained from the subject has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor-infiltrating immune cells in the tumor sample. For example, in some embodiments, a tumor sample obtained from the subject has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor-infiltrating immune cells in the tumor sample.

In other embodiments, a tumor sample obtained from the subject has been determined to have a detectable expression level of PD-L1 in tumor-infiltrating immune cells that comprise about 5% or more of the tumor sample. For example, in some embodiments, a tumor sample obtained from the subject has been determined to have a detectable expression level of PD-L1 in tumor-infiltrating immune cells that comprise from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor sample.

In yet other embodiments, a tumor sample obtained from the subject has been determined to have a detectable expression level of PD-L1 in about 5% or more of the tumor-infiltrating immune cells in the tumor sample. For example, in some embodiments, a tumor sample obtained from the subject has been determined to have a detectable expression level of PD-L1 in from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor-infiltrating immune cells in the tumor sample. In still further embodiments, a tumor sample obtained from the subject has been determined to have a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

In still further embodiments, a tumor sample obtained from the subject has been determined to have a detectable expression level of PD-L1 in about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor-infiltrating immune cells in the tumor sample.

In yet other embodiments, a tumor sample obtained from the subject has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample and/or a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some embodiments, the anti-PD-L1 antibody is SP142. In some embodiments, the anti-PD-L1 antibody is SP263.

In some embodiments, a tumor sample obtained from the subject has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. For example, in some embodiments, a tumor sample obtained from the subject has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor cells in the tumor sample. In other embodiments, a tumor sample obtained from the subject has been determined to have a detectable expression level of PD-L1 in less than about 1% of the tumor cells in the tumor sample.

In other embodiments, a tumor sample obtained from the subject has been determined to have a detectable expression level of PD-L1 in about 5% or more of the tumor cells in the tumor sample. For example, in some embodiments, a tumor sample obtained from the subject has been determined to have a detectable expression level of PD-L1 in from about 5% to less than 50% (e.g., from 5% to 49.5%, from 5% to 45%, from 5% to 40%, from 5% to 35%, from 5% to 30%, from 5% to 25%, from 5% to 20%, from 5% to 15%, from 5% to 10%, from 5% to 9%, from 5% to 8%, from 5% to 7%, from 5% to 6%, from 10% to 49.5%, from 10% to 40%, from 10% to 35%, from 10% to 30%, from 10% to 25%, from 10% to 20%, from 10% to 15%, from 15% to 49.5%, from 15% to 45%, from 15% to 40%, from 15% to 35%, from 15% to 30%, from 15% to 30%, from 15% to 25%, from 15% to 20%, from 20% to 49.5%, from 20% to 45%, from 20% to 40%, from 20% to 35%, from 20% to 30%, from 20% to 25%, from 25% to 49.5%, from 25% to 45%, from 25% to 40%, from 25% to 35%, from 25% to 30%, from 30% to 49.5%, from 30% to 45%, from 30% to 40%, from 30% to 35%, from 35% to 49.5%, from 35% to 45%, from 35% to 40%, from 40% to 49.5%, from 40% to 45%, or from 45% to 49.5%) of the tumor cells in the tumor sample.

In yet other embodiments, a tumor sample obtained from the subject has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. In some embodiments, a tumor sample obtained from the subject has been determined to have a detectable expression level of PD-L1 in from about 50% to about 99% (e.g., from 50% to 99%, from 50% to 95%, from 50% to 90%, from 50% to 85%, from 50% to 80%, from 50% to 75%, from 50% to 70%, from 50% to 65%, from 50% to 60%, from 50% to 55%, from 55% to 99%, from 55% to 95%, from 55% to 90%, from 55% to 85%, from 55% to 80%, from 55% to 75%, from 55% to 70%, from 55% to 65%, from 55% to 60%, from 60% to 99%, from 60% to 95%, from 60% to 90%, from 60% to 85%, from 60% to 80%, from 60% to 75%, from 60% to 70%, from 60% to 65%, from 65% to 99%, from 65% to 95%, from 65% to 90%, from 65% to 85%, from 65% to 80%, from 65% to 75%, from 65% to 70%, from 70% to 99%, from 70% to 95%, from 70% to 90%, from 70% to 85%, from 70% to 80%, from 70% to 75%, from 75% to 99%, from 75% to 95%, from 75% to 90%, from 75% to 85%, from 75% to 80%, from 80% to 99%, from 80% to 95%, from 80% to 90%, from 80% to 85%, from 85% to 99%, from 85% to 95%, from 85% to 90%, from 90% to 99%, or from 90% to 95%) of the tumor cells in the tumor sample.

In some embodiments, the tumor sample is a formalin-fixed and paraffin-embedded (FFPE) tumor sample, an archival tumor sample, a fresh tumor sample, or a frozen tumor sample.

The presence and/or expression level of any of the biomarkers described above (including PD-L1 (e.g., PD-L1 expression on tumor-infiltrating immune cells (IC) in a tumor sample obtained from the subject and/or PD-L1 expression on tumor cells (TC) in a tumor sample obtained from the subject)), e.g., in a tumor sample obtained from the subject) may be assessed qualitatively and/or quantitatively based on any suitable criterion known in the art, including but not limited to DNA, mRNA, cDNA, proteins, protein fragments, and/or gene copy number. Methodologies for measuring such biomarkers are known in the art and understood by the skilled artisan, including, but not limited to, IHC, Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNASeq, microarray analysis, gene expression profiling, whole-genome sequencing (WGS), and/or serial analysis of gene expression (“SAGE”), as well as any one of the wide variety of assays that can be performed by protein, gene, and/or tissue array analysis. Typical protocols for evaluating the status of genes and gene products are found, for example, in Ausubel et al. eds. (Current Protocols in Molecular Biology, 1995), Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments of any of the preceding methods, the expression level of a biomarker (e.g., PD-L1) may be a protein expression level. In certain embodiments, the method comprises contacting the sample with antibodies that specifically bind to a biomarker described herein under conditions permissive for binding of the biomarker, and detecting whether a complex is formed between the antibodies and biomarker. Such method may be an in vitro or in vivo method. In some embodiments, an antibody is used to select subjects eligible for treatment with an anti-cancer therapy that includes a PD-1 axis binding antagonist, e.g., an anti-PD-L1 antibody (e.g., atezolizumab) or an anti-PD-1 antibody, e.g., a biomarker for selection of subjects. In some embodiments, an antibody is used to select subjects eligible for treatment with an anti-cancer therapy that includes a PD-1 axis binding antagonist (e.g., an anti-PD-L1 antibody (e.g., atezolizumab) or an anti-PD-1 antibody) and an IL6 antagonist (e.g., anti-IL6 receptor antibody such as tocilizumab), e.g., a biomarker for selection of subjects.

Any method of measuring protein expression levels known in the art or provided herein may be used. For example, in some embodiments, a protein expression level of a biomarker is determined using a method selected from the group consisting of immunohistochemistry (IHC), flow cytometry (e.g., fluorescence-activated cell sorting (FACS™)), Western blot, enzyme-linked immunosorbent assay (ELISA), immunoprecipitation, immunofluorescence, radioimmunoassay, dot blotting, immunodetection methods, HPLC, surface plasmon resonance, optical spectroscopy, mass spectrometry, and HPLC.

In some embodiments, the protein expression level of the biomarker (e.g., PD-L1) is determined in tumor-infiltrating immune cells. In some embodiments, the protein expression level of the biomarker is determined in tumor cells. In some embodiments, the protein expression level of the biomarker is determined in tumor-infiltrating immune cells and/or in tumor cells. In some embodiments, the protein expression level of the biomarker is determined in peripheral blood mononuclear cells (PBMCs).

In certain embodiments, the presence and/or expression level/amount of a biomarker protein (e.g., PD-L1) in a sample is examined using IHC and staining protocols. IHC staining of tissue sections has been shown to be a reliable method of determining or detecting the presence of proteins in a sample. In some embodiments of any of the methods, assays and/or kits, the biomarker is one or more of the protein expression products of PD-L1. In one embodiment, an expression level of biomarker is determined using a method comprising: (a) performing IHC analysis of a sample (such as a tumor sample obtained from a subject) with an antibody; and (b) determining expression level of a biomarker in the sample. In some embodiments, IHC staining intensity is determined relative to a reference. In some embodiments, the reference is a reference value. In some embodiments, the reference is a reference sample (e.g., a control cell line staining sample, a tissue sample from non-cancerous subject, or a tumor sample that is determined to be negative for the biomarker of interest).

For example, in some embodiments, the protein expression level of PD-L1 is determined using IHC. In some embodiments, the protein expression level of PD-L1 is detected using an anti-PD-L1 antibody. Any suitable anti-PD-L1 antibody may be used, including, e.g., SP142, SP263, 22C3, 28-8, E1L3N, 4059, h5H1, and 9A11. In some embodiments, the anti-PD-L1 antibody is SP142. In some embodiments, the anti-PD-L1 antibody is SP263.

IHC may be performed in combination with additional techniques such as morphological staining and/or in situ hybridization (e.g., ISH). Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) radioisotopes, such as 35S, 14C, 1251, 3 H, and¹³¹1; (b) colloidal gold particles; (c) fluorescent labels including, but are not limited to, rare earth chelates (europium chelates), Texas Red, rhodamine, fluorescein, dansyl, lissamine, umbelliferone, phycocrytherin, phycocyanin, or commercially-available fluorophores such as SPECTRUM ORANGE® and SPECTRUM GREEN® and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; see, e.g., U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). For a general review of these, see, for example, U.S. Pat. Nos. 4,275,149 and 4,318,980.

Specimens may be prepared, for example, manually, or using an automated staining instrument (e.g., a Ventana BenchMark XT or Benchmark ULTRA instrument). Specimens thus prepared may be mounted and coverslipped. Slide evaluation is then determined, for example, using a microscope, and staining intensity criteria, routinely used in the art, may be employed. In one embodiment, it is to be understood that when cells and/or tissue from a tumor is examined using IHC, staining can be determined or assessed in tumor cell(s) and/or tissue (as opposed to stromal or surrounding tissue that may be present in the sample). In other embodiments, staining can be determined or assessed in stromal or surrounding tissue that may be present in the sample. In some embodiments, it is understood that when cells and/or tissue from a tumor is examined using IHC, staining includes determining or assessing in tumor-infiltrating immune cells, including intratumoral or peritumoral immune cells. In some embodiments, the presence of a biomarker is detected by IHC in >0% of the sample, in at least 1% of the sample, in at least 5% of the sample, in at least 10% of the sample, in at least 15% of the sample, in at least 15% of the sample, in at least 20% of the sample, in at least 25% of the sample, in at least 30% of the sample, in at least 35% of the sample, in at least 40% of the sample, in at least 45% of the sample, in at least 50% of the sample, in at least 55% of the sample, in at least 60% of the sample, in at least 65% of the sample, in at least 70% of the sample, in at least 75% of the sample, in at least 80% of the sample, in at least 85% of the sample, in at least 90% of the sample, in at least 95% of the sample, or more. Samples may be scored using any method known in the art, for example, by a pathologist or automated image analysis.

In some embodiments of any of the methods, the biomarker is detected by immunohistochemistry using a diagnostic antibody (i.e., primary antibody). In some embodiments, the diagnostic antibody specifically binds human antigen. In some embodiments, the diagnostic antibody is a non-human antibody. In some embodiments, the diagnostic antibody is a rat, mouse, or rabbit antibody. In some embodiments, the diagnostic antibody is a rabbit antibody. In some embodiments, the diagnostic antibody is a monoclonal antibody. In some embodiments, the diagnostic antibody is directly labeled. In other embodiments, the diagnostic antibody is indirectly labeled (e.g., by a secondary antibody).

In other embodiments of any of the preceding methods, the expression level of a biomarker may be a nucleic acid expression level (e.g., a DNA expression level or an RNA expression level (e.g., an mRNA expression level)). Any suitable method of determining a nucleic acid expression level may be used. In some embodiments, the nucleic acid expression level is determined using RNAseq, RT-qPCR, qPCR, multiplex qPCR or RT-qPCR, microarray analysis, SAGE, MassARRAY technique, ISH, or a combination thereof.

Methods for the evaluation of mRNAs in cells are well known and include, for example, serial analysis of gene expression (SAGE), whole genome sequencing (WGS), hybridization assays using complementary DNA probes (such as in situ hybridization using labeled riboprobes specific for the one or more genes, Northern blot and related techniques) and various nucleic acid amplification assays (such as RT-PCR (e.g., qRT-PCR) using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising an immunotherapy and a suppressive stromal antagonist may be arrayed on a solid support. Hybridization of a labeled probe with a particular array member indicates that the sample from which the probe was derived expresses that gene.

The sample may be obtained from the subject at any suitable time. For example, in some embodiments, the sample is obtained from the subject prior to (e.g., minutes, hours, days, weeks (e.g., 1, 2, 3, 4, 5, 6, or 7 weeks), months, or years prior to) administration of the treatment regimen. In some embodiments of any of the preceding methods, the sample from the subject is obtained about 2 to about 10 weeks (e.g., 2, 3, 4, 5, 6, 7, 8, 9, or 10 weeks) following administration of the treatment regimen. In some embodiments, the sample from the subject is obtained about 4 to about 6 weeks following administration of the treatment regimen.

In some embodiments, the expression level or number of a biomarker (e.g., PD-L1) is detected in a tissue sample, a primary or cultured cells or cell line, a cell supernatant, a cell lysate, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, or any combination thereof. In some embodiments, the sample is a tissue sample (e.g., a tumor tissue sample), a cell sample, a whole blood sample, a plasma sample, a serum sample, or a combination thereof. In some embodiments, the tumor tissue sample wherein the tumor tissue sample includes tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample.

For example, in some embodiments, the expression level of a biomarker (e.g., PD-L1) is detected in tumor-infiltrating immune cells, tumor cells, PBMCs, or combinations thereof using known techniques (e.g., IHC, immunofluorescence microscopy, or flow cytometry). Tumor-infiltrating immune cells include, but are not limited to, intratumoral immune cells, peritumoral immune cells or any combinations thereof, and other tumor stroma cells (e.g., fibroblasts). Such tumor infiltrating immune cells may be T lymphocytes (such as CD8⁺ T lymphocytes (e.g., CD8⁺ T effector (Teff) cells) and/or CD4⁺ T lymphocytes (e.g., CD4⁺ Teff cells), B lymphocytes, or other bone marrow-lineage cells including granulocytes (neutrophils, eosinophils, basophils), monocytes, macrophages, dendritic cells (e.g., interdigitating dendritic cells), histiocytes, and natural killer (NK) cells. In some embodiments, the staining for a biomarker is detected as membrane staining, cytoplasmic staining, or combinations thereof. In other embodiments, the absence of a biomarker is detected as absent or no staining in the sample, relative to a reference sample.

In particular embodiments, the expression level of a biomarker is assessed in a sample that contains or is suspected to contain cancer cells. The sample may be, for example, a tissue biopsy or a metastatic lesion obtained from a subject suffering from, suspected to suffer from, or diagnosed with cancer (e.g., bladder cancer (e.g., UC, including locally advanced or metastatic UC). In some embodiments, the sample is a sample of tissue (e.g., renal pelvis, ureter, urinary bladder, and/or urethral tissue), a biopsy of a tumor (e.g., a locally advanced or metastatic UC tumor, including a pelvis, ureter, urinary bladder, and/or urethral tumor), a known or suspected metastatic bladder cancer (e.g., metastatic UC) lesion or section, or a blood sample, e.g., a peripheral blood sample, known or suspected to comprise circulating cancer cells, e.g., bladder cancer cells (e.g., UC cells, including locally advanced or metastatic UC cells). The sample may comprise both cancer cells, i.e., tumor cells, and non-cancerous cells (e.g., lymphocytes, such as T cells or NK cells), and, in certain embodiments, comprises both cancerous and non-cancerous cells. Methods of obtaining biological samples including tissue resections, biopsies, and body fluids, e.g., blood samples comprising cancer/tumor cells, are well known in the art.

In certain embodiments, the subject may have an advanced, refractory, recurrent, and/or chemotherapy-resistant form of the cancer.

In certain embodiments, the presence and/or expression levels/amount of a biomarker in a first sample is increased or elevated as compared to presence/absence and/or expression levels/amount in a second sample. In certain embodiments, the presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue.

In certain embodiments, a reference sample, reference cell, reference tissue, control sample, control cell, or control tissue is a single sample or combined multiple samples from the same subject that are obtained at one or more different time points than when the test sample is obtained. For example, a reference sample, reference cell, reference tissue, control sample, control cell, or control tissue is obtained at an earlier time point from the same subject than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the subject. In certain embodiments, a reference sample, reference cell, reference tissue, control sample, control cell, or control tissue is a combined multiple samples from one or more individuals with a disease or disorder (e.g., cancer) who are not the subject. In certain embodiments, a reference sample, reference cell, reference tissue, control sample, control cell, or control tissue is pooled RNA samples from normal tissues or pooled plasma or serum samples from one or more individuals who are not the subject. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the subject.

In some embodiments, the method further includes administering an effective amount of a treatment regimen described herein (e.g., a treatment regimen comprising a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) to the subject, for example, based on the expression level of one or more biomarkers (e.g., PD-L1).

### VIII. Pharmaceutical Compositions and Formulations

Also provided herein are pharmaceutical compositions and formulations comprising a PD-1 axis binding antagonist and/or an antibody described herein (such as an anti-PD-L1 antibody (e.g., atezolizumab) or an anti-PD-1 antibody) and, optionally, a pharmaceutically acceptable carrier. The invention also provides pharmaceutical compositions and formulations comprising an IL6 antagonist (e.g., an anti-IL6 receptor antibody such as tocilizumab), and optionally, a pharmaceutically acceptable carrier.

Pharmaceutical compositions and formulations as described herein can be prepared by mixing the active ingredients (e.g., a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The compositions and formulations herein may also contain more than one active ingredient as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films or microcapsules. The formulations to be used for in vivo administration are generally sterile. Sterility may be readily accomplished, e.g., by filtration through sterile filtration membranes.

### IX. Articles of Manufacture or Kits

In another embodiment of the invention, an article of manufacture or a kit is provided comprising a PD-1 axis binding antagonist (e.g., an anti-PD-L1 antibody (e.g., atezolizumab) or an anti-PD-1 antibody) and/or an IL6 antagonist (e.g., anti-IL6 receptor antibody such as tocilizumab). In some embodiments, the article of manufacture or kit further comprises package insert comprising instructions for using the PD-1 axis binding antagonist to treat cancer, e.g. breast or urothelial carcinoma. In some embodiments, the article of manufacture or kit further comprises package insert comprising instructions for using the PD-1 axis binding antagonist in combination with an IL6 antagonist (e.g. an anti-IL6 receptor antibody (e.g. tocilizumab)) to treat or the cancer. Any of the PD-1 axis binding antagonists and/or IL6 antagonists described herein may be included in the article of manufacture or kits.

In some embodiments, the PD-1 axis binding antagonist (e.g., an anti-PD-L1 antibody (e.g., atezolizumab) or an anti-PD-1 antibody) and the IL6 antagonist (e.g., anti-IL6 receptor antibody such as tocilizumab) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., an additional chemotherapeutic agent or anti-neoplastic agent). Suitable containers for the one or more agent include, for example, bottles, vials, bags and syringes.

In another example, provided herein is a kit for treating breast cancer (e.g. TNBC) or urothelial carcinoma or renal cell carcinoma in a subject in need thereof, the kit comprising a PD-1 axis binding antagonist (e.g., an anti-PD-L1 antibody (e.g., atezolizumab) or an anti-PD-1 antibody) and/or an IL6 antagonist (e.g. an anti-IL6 receptor antibody (e.g. tocilizumab)) and instructions for administering the PD-1 axis binding antagonist and/or the IL6 antagonist to a patient with CRP and/or IL-6 level(s) above the upper limit of normal and/or PD-L1 expression.

The specification is considered to be sufficient to enable one skilled in the art to practice the invention. Various modifications of the invention in addition to those shown and described herein will become apparent to those skilled in the art from the foregoing description and fall within the scope of the appended claims.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Correlations Between Biomarkers of Systemic Inflammation and Outcome in Patients with Metastatic Triple-Negative Breast Cancer (mTNBC) Treated with Atezolizumab Monotherapy

Background:

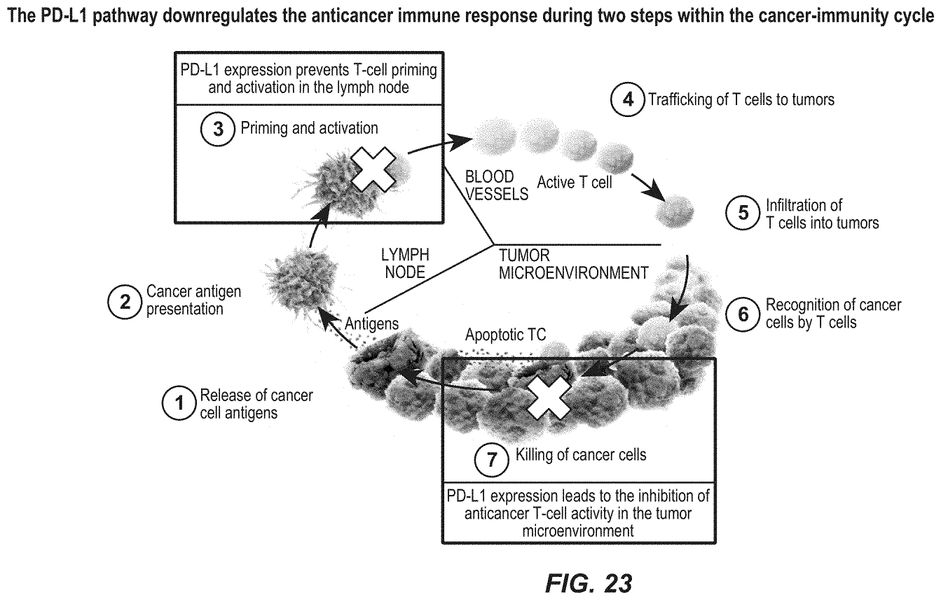

FIG. 23 depicts schematically how the PD-L1 pathway downregulates the anticancer immune response during the two steps within the cancer-immunity cycle. Immune checkpoint blockade by anti-PD-L1 antibody atezolizumab has demonstrated clinical benefits in metastatic triple negative breast cancer (mTNBC). IL-6 and IL-8 myeloid inflammation is linked to poor prognosis in cancer patients treated with chemotherapy, but its association with single agent atezolizumab-treated patients with mTNBC remains unknown. In this study, we investigated the association of biomarkers (BM) of systemic inflammation with clinical outcomes in patients with mTNBC treated with atezolizumab monotherapy.

Among breast cancer subtypes, TNBC has the worst outcomes. Historically, chemotherapy has been the typical treatment for metastatic or advanced disease. Estimates of median OS for mTNBC vary but are generally around 18 months or less. Pre-existing tumor immune biology (including PD-L1 expression in the tumor microenvironment, CD8⁺ T cells, stromal TILs) has been associated with clinical activity in mTNBC in patients treated with PD-L1/PD-1-targeting agents, including the anti-PD-L1 checkpoint inhibitor atezolizumab. Single-agent anti-PD-L1/PD-1 mAbs are active in mTNBC but to a lower extent vs ORRs with standard-of-care chemotherapy.

CIT combination options are being evaluated to improve outcomes in this disease Phase III IMpassion130 study: atezolizumab+nab-paclitaxel was the first CIT combination demonstrating clinical benefit (PFS/OS) in the 1L setting in patients with TNBC expressing PD-L1 on IC leading to FDA accelerated approval in this setting. Proof-of-concept combination of atezolizumab+(nab)paclitaxel+ipatasertib shows confirmed ORR of 73% (19/26 patients) in biomarker un-selected 1L mTNBC.

A deep dive into the biology of atezolizumab monotherapy-treated patients with TNBC may lead to new CIT combination options in this difficult-to-treat patient population.

Methods:

Baseline pre-treatment plasma samples were collected from mTNBC patients enrolled in the phase I clinical trial PCD4989g (NCT01375842, atezolizumab monotherapy, n=113). IL-6, IL-8 and CRP were tested by Luminex assays and their levels were assessed for association with baseline clinical demographic characteristics and atezolizumab clinical activity for response rate (ORR), progression free (PFS) and overall survival (OS).

Target Population:

patients with TNBC in a Phase Ia study expansion cohort (N=116)
1L, 2L, 3L+ settings
Initially enrolled only PD-L1-high patients and later expanded to include all-comers^{a}

Treatment:
atezolizumab IV q3w at 15 or 20 mg/kg or 1200-mg flat dose

Objectives:
Primary endpoint: safety
Key secondary endpoints: ORR, DOR and PFS (per RECIST v1.1 and irRC)
Key exploratory endpoints: OS and biomarkers of clinical activity
   Plasma biomarkers tested by ELISA or Luminex assay

Results:

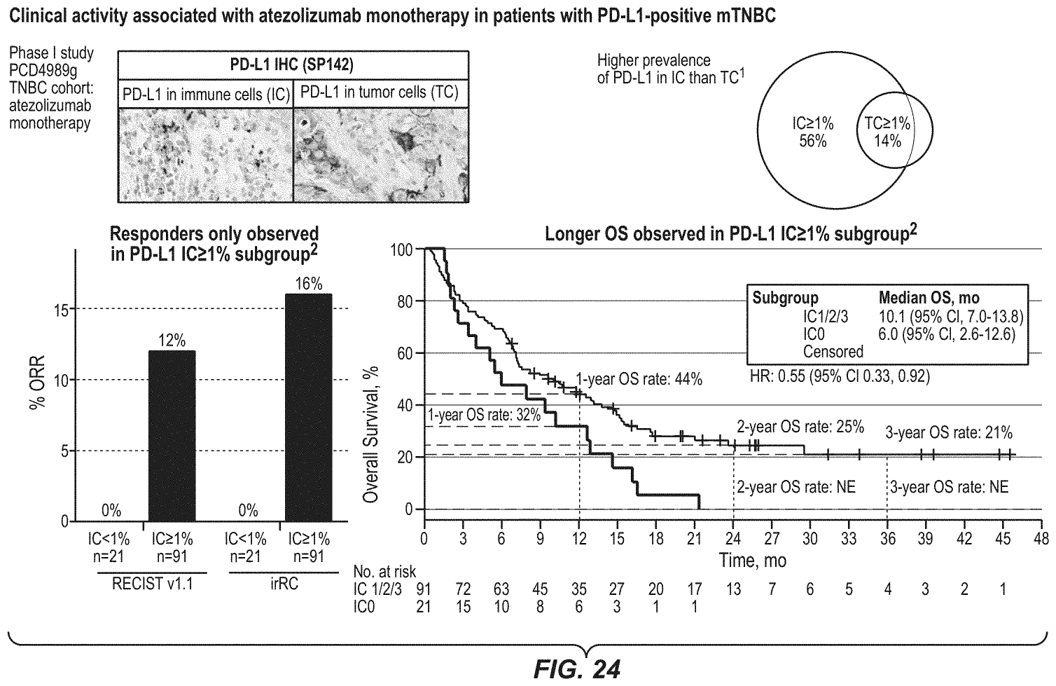

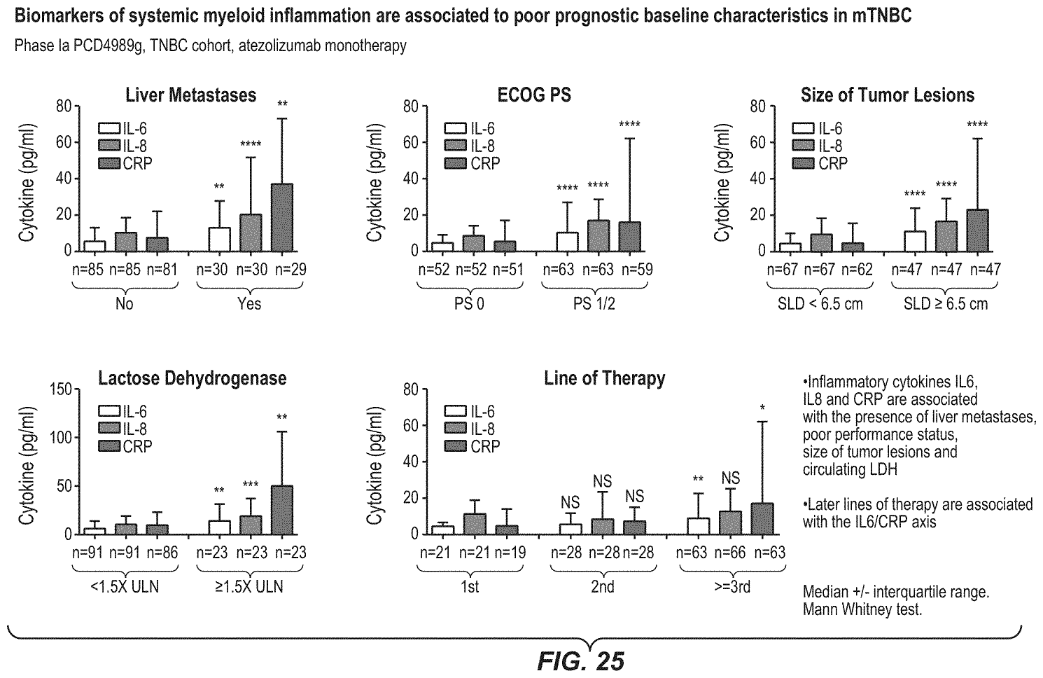

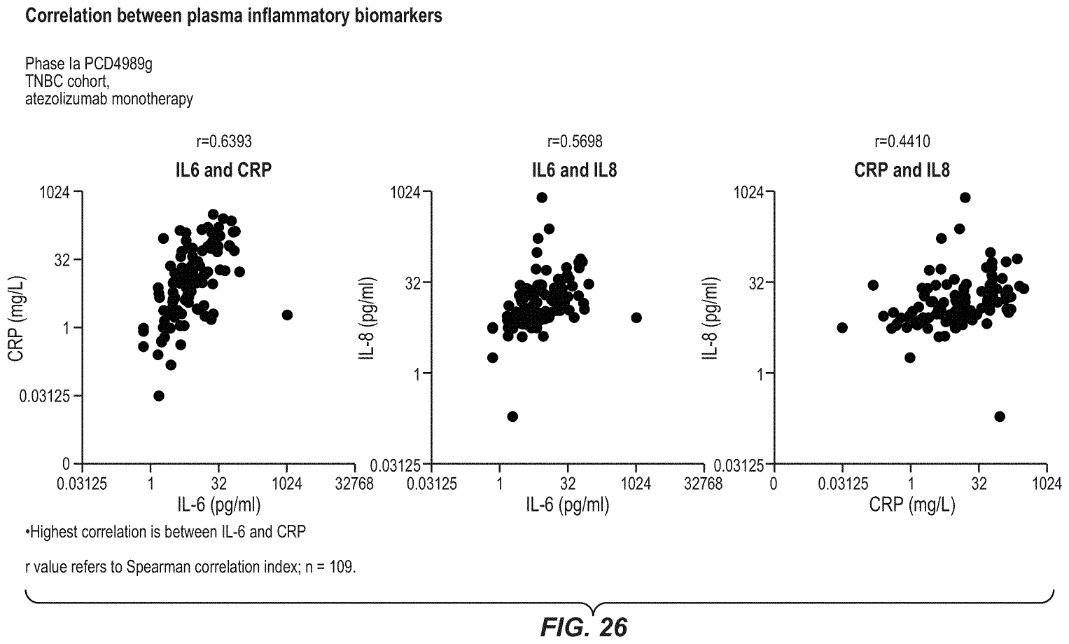

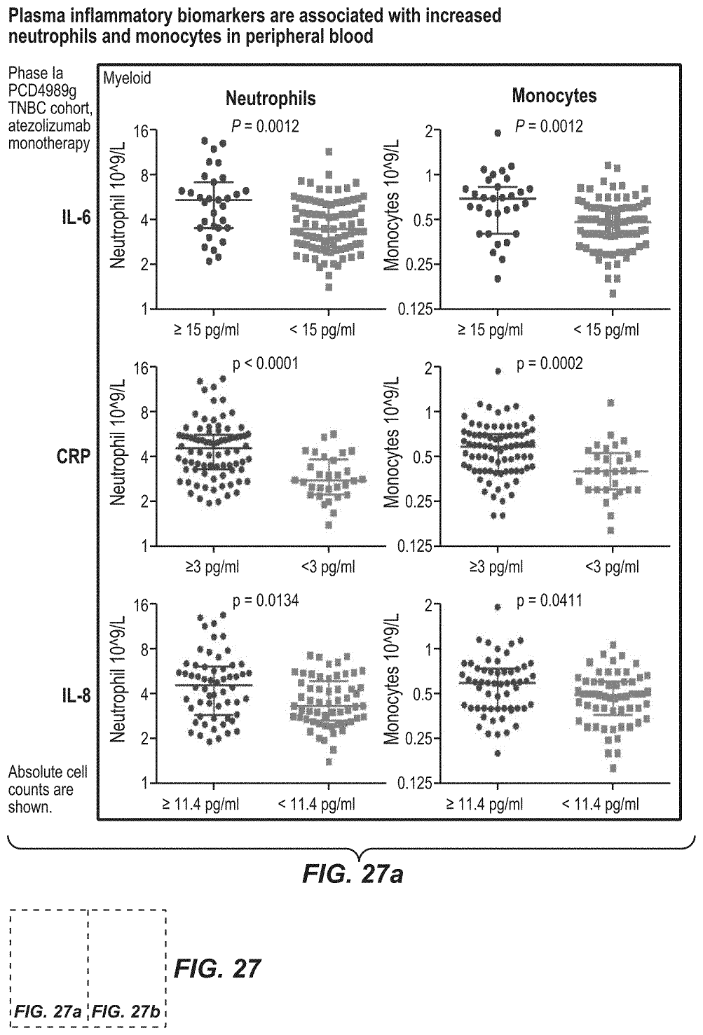

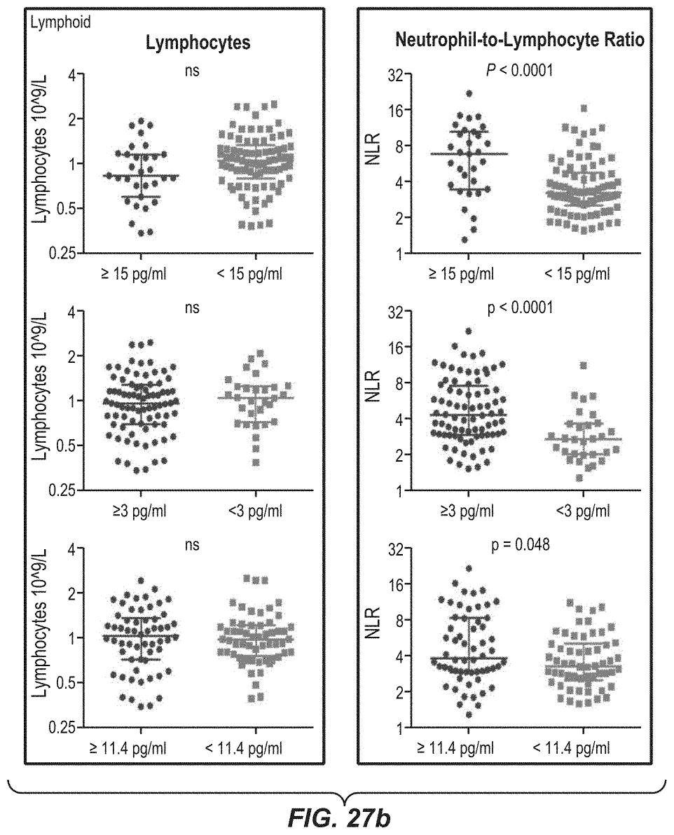

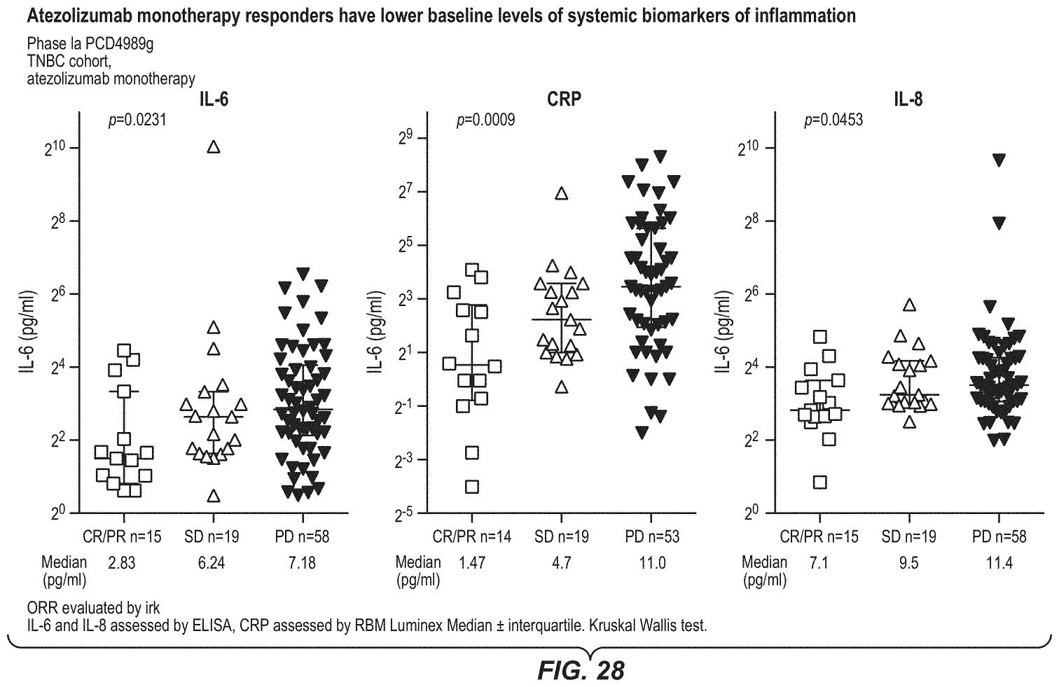

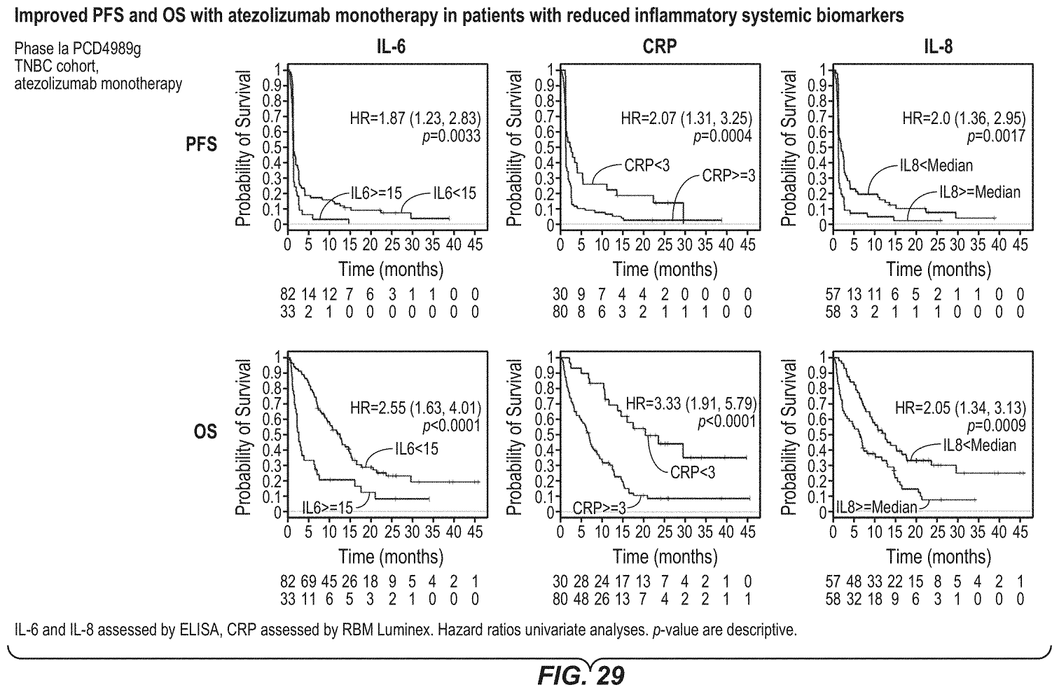

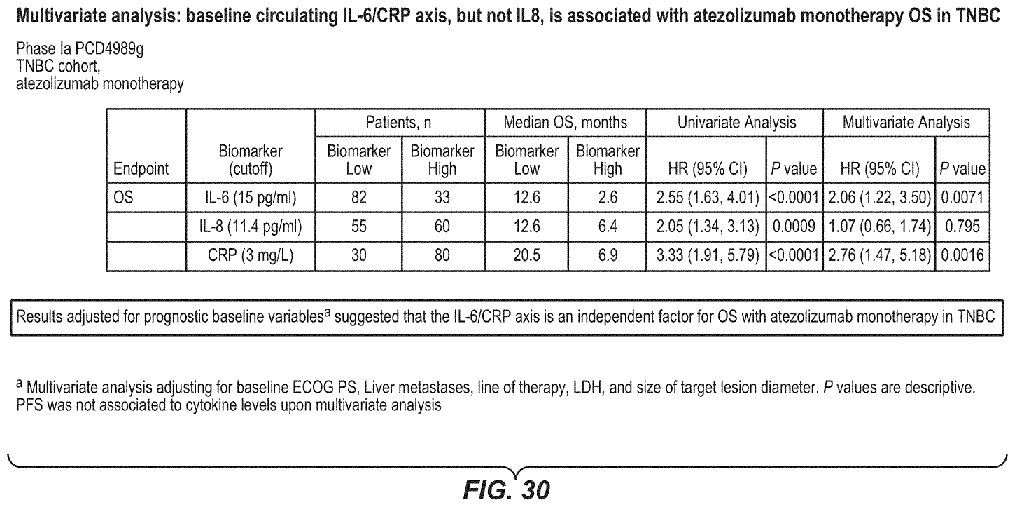

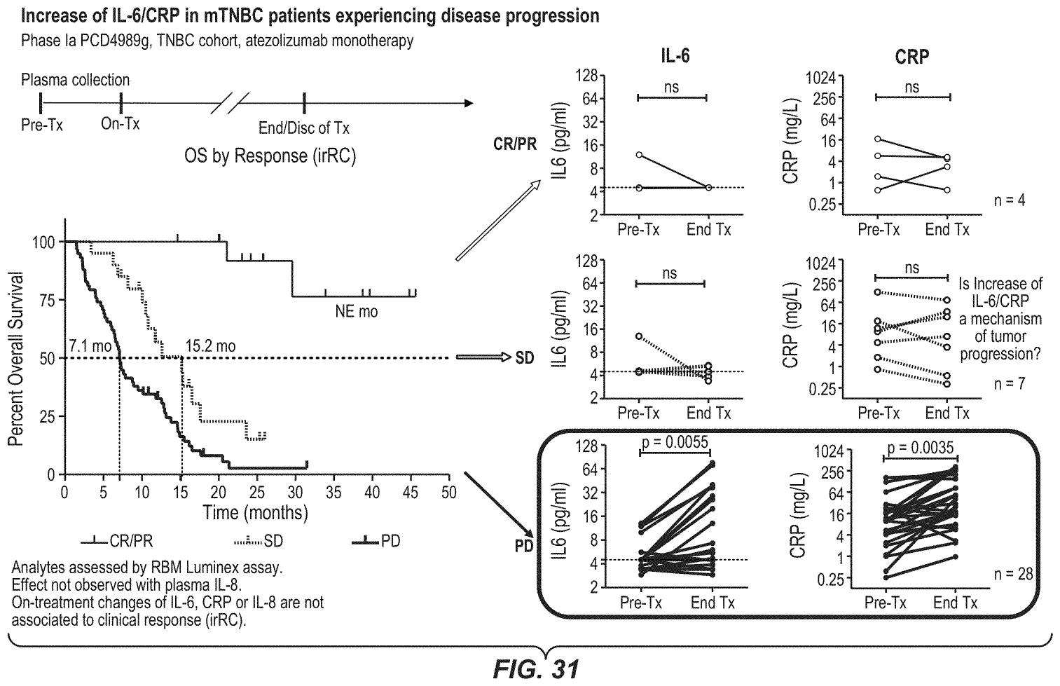

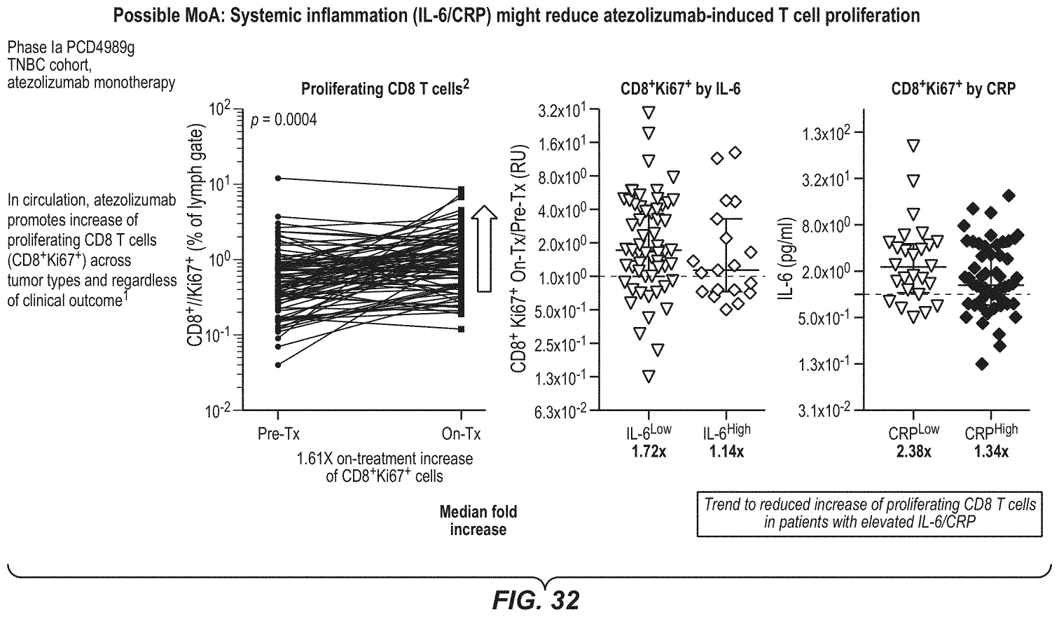

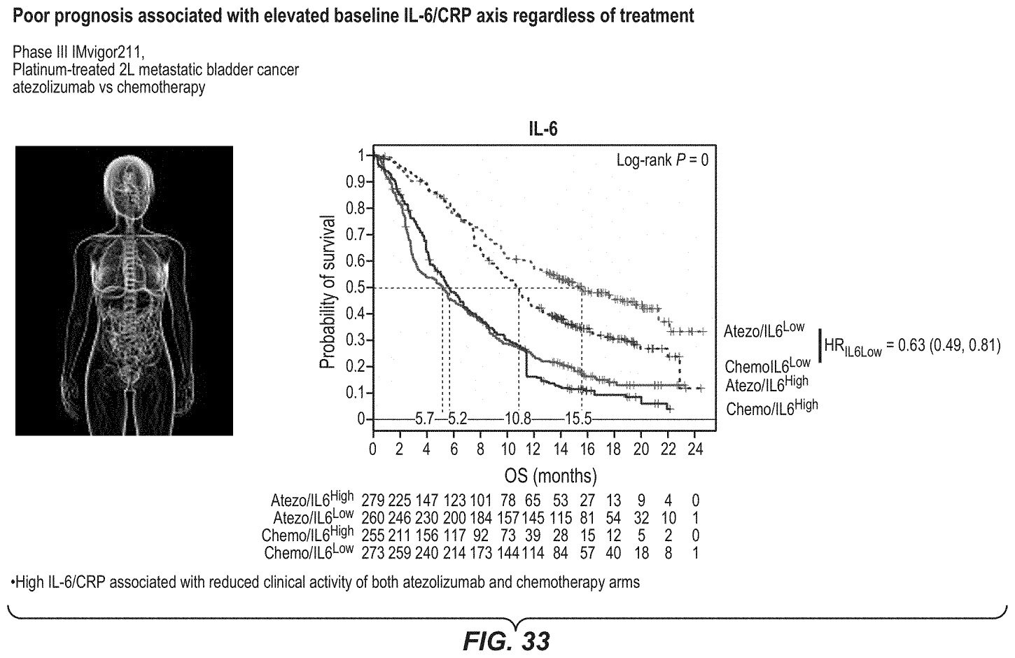

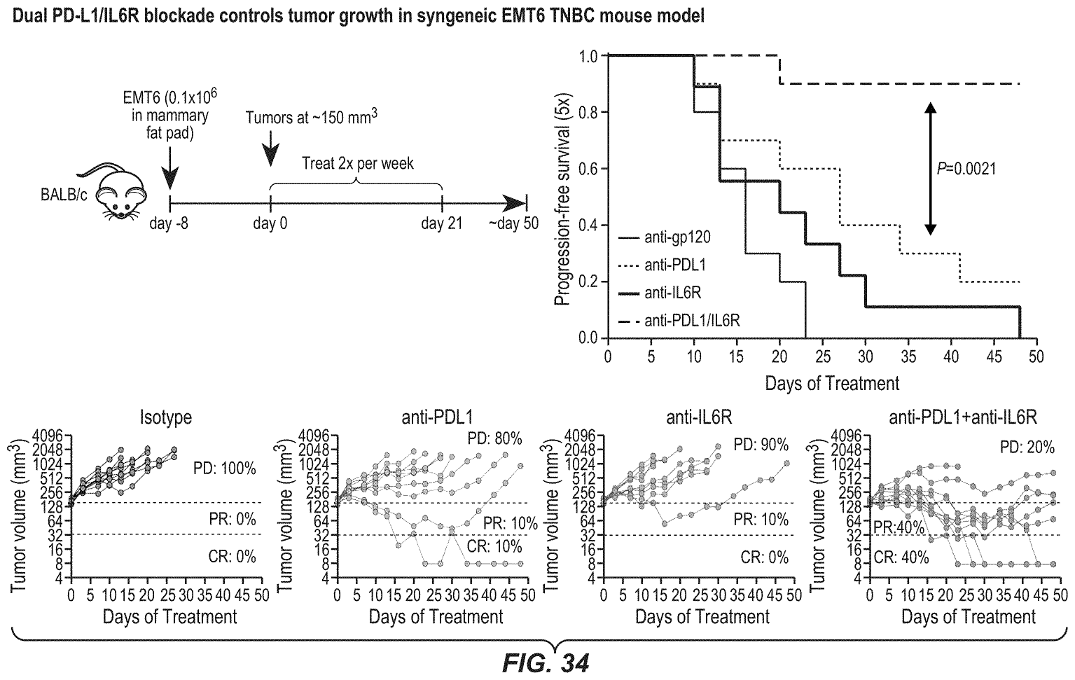

The results are depicted in FIGS. 24-34. Baseline IL-6, IL-8, and CRP levels were positively associated with the clinical prognostic traits ECOG performance status (>0), presence of liver metastases, large size of target lesions (≥5 cm), and increased LDH (≥1.5×ULN) (FIG. 25). Elevated baseline IL-6 and CRP, but not IL-8, were linked with later lines of therapy (2) (FIG. 26). Univariate analyses showed that IL-6 (≥15 pg/ml), IL-8 (≥11.4 pg/ml) and CRP (≥3 mg/L) were associated with reduced OS and PFS, but only CRP was associated to reduced ORR (Fig. Multivariate analyses considering the aforementioned clinical demographic variables showed that IL-6 (HR 2.00 [1.16-3.38]); and CRP (HR 2.74 [1.49-5.28]), but not IL-8 (HR 1.07 [0.78-1.74]), were associated with OS (FIG. 30).

Conclusion:

The IL-6/CRP inflammatory axis is an independent factor linked with poor outcomes of mTNBC patients treated with atezolizumab monotherapy and may play unique roles in affecting the anti-tumor activities.

The Objective of this exploratory, signal seeking analysis based on data from a phase I monotherapy study was to identify new potential drug targets that might be relevant for future drug combinations in CIT. The retrospective analysis in this Phase Ia of atezolizumab monotherapy-treated patients with TNBC is consistent with the poor prognosis associated with systemic myeloid inflammation in patients treated with chemotherapy or single-agent immune checkpoint inhibitors. The data presented indicates that:
Increased biomarkers of systemic inflammation are linked to poor prognostic characteristics at baseline.
The systemic IL-6/CRP axis, but not IL-8, is an independent variable associated with OS in patients with mTNBC treated with atezolizumab monotherapy.
Possible mechanism of tumor progression: increase IL6/CRP inflammatory axis.
Dual blockade of PD-L1 and IL-6R leads to improved tumor growth control in a TNBC mouse model

### Implications:

In patients with TNBC and systemic inflammation, combined blockade of IL-6R and PD-L1 can improve clinical outcome.

### Example 2: Materials and Methods for Examples 3 to 6

Clinical Tumor Sample Collection:

TNBC tumor samples for this analysis were collected from PCD4989g (NCT01375842), a single-arm Phase I study that evaluated the clinical activity of atezolizumab in patients with locally advanced or metastatic malignancies, including TNBC. Bladder cancer tumor samples were collected in IMvigor210, a single-arm Phase 2 study investigating atezolizumab in mUC patients (NCT02951767, NCT02108652) and in Phase 3 mUC trial IMvigor211 (NCT02302807) in which patients were treated with either chemotherapy (taxane or vinflunine) or atezolizumab as a second-line or higher treatment. Tumor tissues were taken from all patients two years prior to study entry. RCC samples were collected from IMmotion150 (NCT01984242), a phase II multicenter, randomized, open-label study investigating activity of atezolizumab and atezolizumab+bevacizumab versus sunitinib in metastatic clear cell renal carcinoma. Tumor specimens from patients were acquired <12 months before study treatment.

Plasma IL-6 Assay:

EDTA plasma samples were collected from patients before (PCD4989g, IMmotion150, IMvigor210, IMvigor211) and on cycle 3 day 1 after treatment (IMvigor211) and stored at −80° C. Plasma IL-6 was evaluated by previously qualified immunoassays on a novel multi-analyte platform Simple Plex Ella (Gupta et al. Bioanalysis 8: 2415-2428 (2016)). The samples were diluted twofold in sample diluent and loaded onto the cartridge for data acquisition.

RNAseq Gene Expression Profiling:

Whole-transcriptome profiles were generated using TruSeq RNA Access technology (Illumina). RNA-seq reads were first aligned to ribosomal RNA sequences to remove ribosomal reads. The remaining reads were aligned to the human reference genome (NCBI Build 38) using GSNAP (Wu et al. Methods Mol Biol 1418: 283-334 (2016); Wu et al. Bioinformatics 26: 873-881 (2010)) version 2013-10-10, allowing a maximum of two mismatches per 75 base sequence (parameters: ‘-M 2 -n 10 -B 2 -i 1 -N 1 -w 200000 -E 1-pairmax-rna=200000 -clip-overlap). To quantify gene expression levels, the number of reads mapped to the exons of each RefSeq gene was calculated using the functionality provided by the R/Bioconductor package GenomicAlignments. The CD8 T cell gene expression signature (GES) was defined in a previous publication for mRCC (McDermott et al. Nat Med 24: 749-757 (2018)).

PBMC Collection and Isolation:

PBMCs from patients were isolated using 50 ml Leucosep™ tubes (Greiner Bio-One International, Germany) and Ficoll-Paque™ PLUS (GE Healthcare, Sweden). Whole blood drawn into sodium heparin blood collection tubes was diluted 3× with phosphate-buffered saline (PBS) without calcium or magnesium (Lonza, Walkersville, Md.). Diluted cell suspensions were carefully layered on Leucosep tubes and centrifuged for 15 minutes at 800×g at room temperature (RT). Interphases containing PBMCs were harvested and washed with PBS and subsequently centrifuged for 10 minutes at 250×g at RT before further processing.

PBMC scRNAseq Library Preparation:

Frozen PBMC samples from mUC patients containing at least 1 million cells were thawed for 1 minute at 37° C. and washed twice with RPMI complete media (10% FBS with glutamate and Pen/Strep). Samples with >50% red blood cells were treated with RBC Lysis buffer for 3 minutes in room temperature to remove red blood cells and then washed one more time with RPMI complete media. The cell density and viability of the single-cell suspension were then determined by Vi-CELL XR cell counter (Beckman Coulter, Pasadena, Calif.). All of the samples had >80% viable cells. Sample processing for single-cell RNA-seq was done using Chromium Single Cell 3' Library and Gel bead kit v2 (PN-120237) following manufacturer's user guide (CG00052, 10× Genomics, Pleasanton, Calif.). The total cell density was used to impute the volume of single cell suspension needed in the reverse transcription (RT) master mix, aiming to achieve ˜6,000 cells per sample. cDNAs and libraries were prepared following manufacturer's user guide (10× Genomics). cDNA amplification and indexed libraries were prepared using 12 and 14 cycles of PCR, respectively. Libraries were profiled, quantified, and sequenced as described above (5′ single-cell gene expression libraries).

scRNAseq Analysis of mUC PBMC:

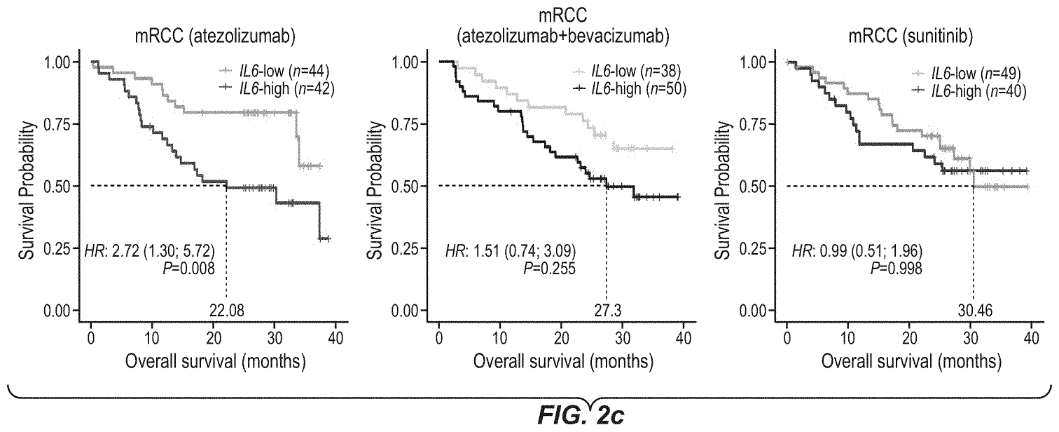

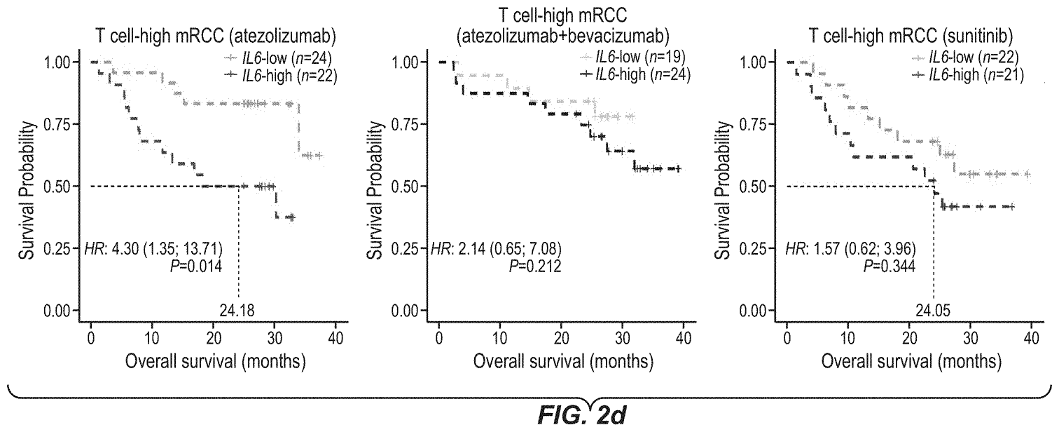

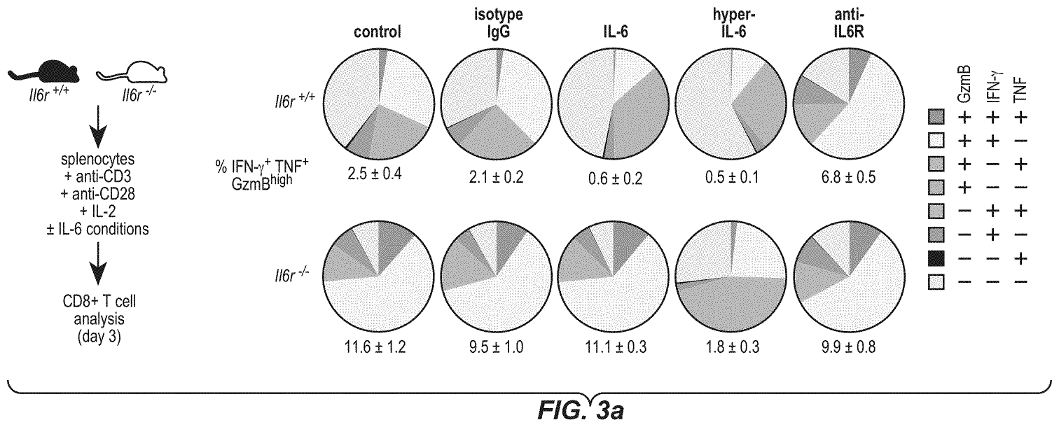

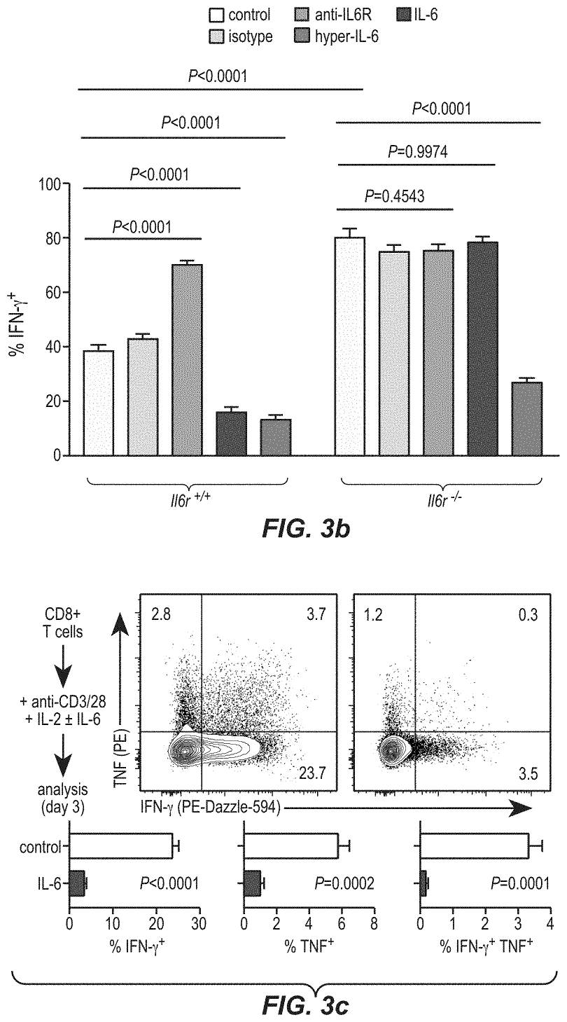

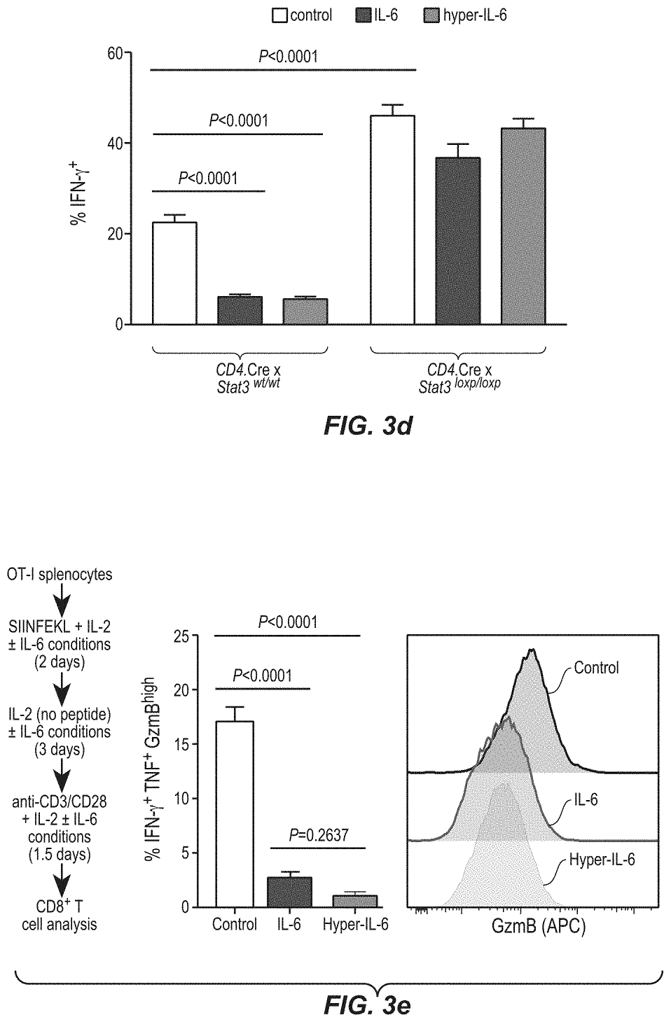

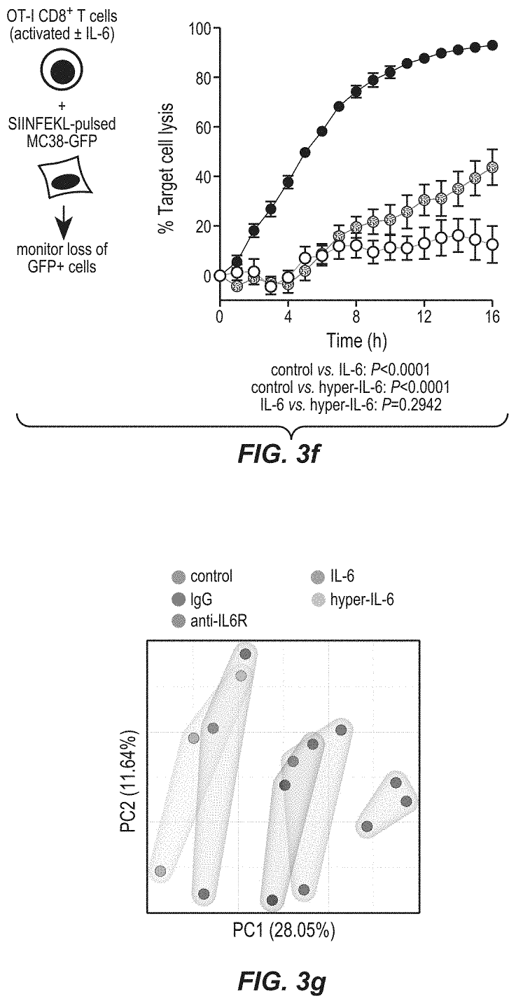

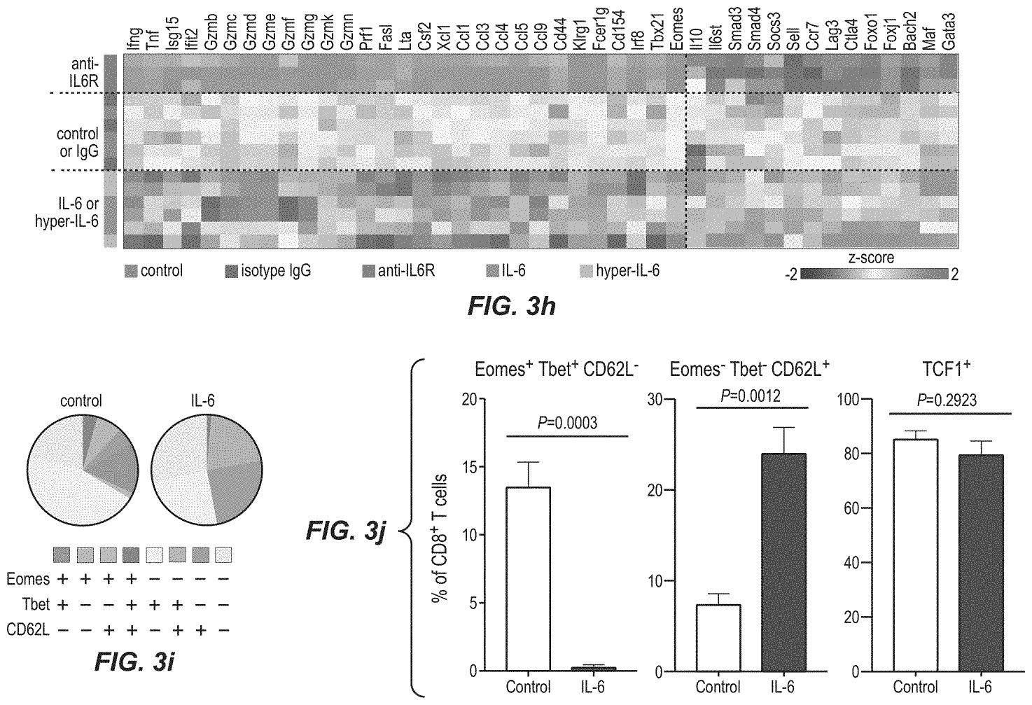

Seurat (Butler et al. Nat Biotechnol 36: 411-420 (2018)) (version 3.0) was used to perform basic quality control on the raw 50 GEX matrices output from Cell Ranger 2.2.0. The Cell Ranger Single Cell Software Suite v.2.2.1 was used to perform sample de-multiplexing, alignment, filtering, and UMI (i.e., universal molecular identifier) counting (https://support.10xgenomics.com/single-cell-gene expression/software/pipelines/latest/what-is-cell-ranger). The data for each respective subpopulation were aggregated for direct comparison of single cell transcriptomes. Then, gene dispersion analysis implemented in Seurat was used to select highly variable genes, preserving genes with logarithmic mean expression between 0.0 and 3.0 and with logarithmic dispersion less than 0.5. Seurat (version 3.0) was used to analyze the PBMC GEX data in FIG. 3. Genes with detected expression in at least five cells, and cells with at least ten genes detected were used. The first 20 principal components were used for clustering (resolution=0.6) and for UMAP visualization. Clusters were identified based on genes that are enriched in a specific cluster. Immunophenotyping of PBMCs was inferred from the annotation of cluster-specific genes; Total T cells (CD3D, CD3E), CD8⁺ T cells (CD3E, CD8A), B cells (CD79A), CD14 monocytes (CD14), and NK cells (NKG7-positive and CD3E-negative).

Differential gene expression analysis for IL-6-high versus IL-6-low cell subsets used raw counts of the samples and was performed by edgeR in R (version 2.13.0,) using the generalized linear model workflow described in the edgeR manual. First, the sequencing reads for duplicate sequencing libraries were combined to produce a single set of sequencing reads for each sample, and the raw read counts for each gene were used to produce a DGEList object in edgeR. Genes were only included if they were represented by at least one read in all of the samples. The calcNormFactors( ) function was used to account for differences in the library size for each sample, and an experimental design model consisting of the batch and HS status was established. The functions estimateCommonDisp( ) and estimateTagwiseDisp( ) were used to estimate dispersion. Following this, differential expression was tested using the exact test based on qCML methods. The Benjamini-Hochberg correction was used with a false discovery cut-off of 0.05.

IL6 In Situ Hybridization:

For the detection of IL6 mRNA expression in RCC tumors, in situ hybridization was performed on 4 um thick formalin-fixed, paraffin-embedded tissue sections mounted on glass slides. The process was automated on the Leica BOND Rx platform (Buffalo Grove, Ill.). A 20 base-pair probe to the target region of IL6 (2-1082) was used (Advanced Cell Diagnostics, Inc., Newark, Calif.). Tissue sections were pre-treated with heat and protease before hybridization with oligonucleotide probes. Detection and amplification was performed with the RNAscope 2.5 LSx Reagent Kit in Red (Advanced Cell Diagnostics, Inc., Newark, Calif.). Tumor sections were analyzed by a qualified histopathologist and considered IL6 positive if at least 1% of either tumor cell area or stromal area showed IL6 stain.

Statistical Analyses of Clinical Data:

Time-to-event outcomes were estimated using the Kaplan-Meier method, which was used to estimate the probability of overall survival (OS) and median overall survival time, and Kaplan-Meier curves. The OS was compared by the log-rank test. For OS analysis, data for patients who were alive were censored at the time of the last contact. The hazard ratios and 95% confidence intervals for OS were estimated by a Cox regression model. Cox proportional hazards and linear regression model was performed to conduct univariate and multivariate analysis.

Software Versions Related to Clinical Data Analysis:

Computational analysis was performed using Cell Ranger software (10× Genomics, Pleasanton, Calif.) version 2.2.0, Perl version 5.18.4, R version 3.6.0, and the following packages and versions in R for analysis: Seurat, 3.0.0; edgeR, 3.26.0; cluster, 2.0.8; dynamicTreeCut, 1.63-1; UMAP, WGCNA, 1.66; and survival, 2.42-6. Figures and tables were generated using the following packages and versions in R: RColorBrewer, 1.1-2; ggplot2, 3.1.1; gridExtra, 2.3; ComplexHeatmap, 2.0.0; superheat, 1.0.0; colorspace, 1.3-2; dplyr, 0.7.8; and data for external datasets were obtained using GenomicDataCommons, 1.4.3; GEOquery, 2.48.0. The above R packages depended secondarily on the following support packages: Matrix, 1.2-17; Biobase, 2.40.0; BiocGenerics, 0.26.0; cowplot, 0.9.3; DDRTree, 0.1.5; edgeR, 2.13.0; irlba, 2.3.2; limma, 3.38.2; magrittr, 1.5; Matrix, 1.2-15; ranger, 0.10.1; and VGAM, 1.0-6.

In Vivo Tumor Studies:

The EMT6 murine mammary carcinoma cell line was obtained from American Type Culture Collection (ATCC; Manassas, Va.), then screened and stored by common cell repository at Genentech. Cell lines are routinely screened and EMT6 cells used in this study were negative for mycoplasma and authenticated by RNA-seq analysis. Cells were cultured in Roswell Park Memorial Institute (RPMI) 1640 medium plus 2 mM L-glutamine with 10% fetal bovine serum (FBS; Hyclone, Waltham, Mass.). Cells in log-phase growth were centrifuged, washed with Hank's balanced salt solution (HBSS), counted, and resuspended in 50% HBSS and 50% Matrigel (BD Biosciences; San Jose, Calif.) at a concentration of 1×10⁶ cells/ml for injection into mice. Female BALB/c mice (8-10 weeks old) were obtained from Charles River Laboratories (Hollister, Calif.) and housed at Genentech in standard rodent microisolator cages. Mice were acclimated for at least 3 days before cell injection. Animals used in this study appeared to be healthy and free of obvious abnormalities. Mice were inoculated in the left #5 mammary fat pad with 1×10⁵ EMT6 cells in 100 μl of HBSS/Matrigel mixture. When tumors reached a volume of 130-230 mm³ (approximately 8 days after inoculation), animals were distributed into treatment groups such that variance in tumor sizes between treatment groups was minimized. Mice were treated with isotype control antibodies, anti-PD-L1 (mouse IgG1 clone 6E11, 10 mg/kg first dose followed by 5 mg/kg thereafter), anti-IL6R (mouse IgG2a clone MR16-1, 15 mg/kg), or a combination of anti-PD-L1 and anti-IL6R. Anti-PD-L1, anti-IL6R, and isotype control antibodies are produced in-house and free of endotoxin contamination. Mice were euthanized after 10-12 days (after 3 doses of treatment) and tumors collected for flow cytometry analysis or IHC. For efficacy studies, antibodies were administered 2 times per week for 3 weeks (intravenously for the first dose and intraperitoneally thereafter). Tumors were measured 2 times per week using digital calipers, and tumor volumes calculated using the modified ellipsoid formula, ½×(length×width²). When tumor volumes fell below 32 mm³ (lower limit of detection) they were considered a complete response (CR; 100% tumor growth inhibition). Tumors that initially regressed but eventually recurred were considered partial responders (PR), and tumors that never regressed were considered to be progressive disease (PD). For time-to-progression analysis, the disease progression endpoint was defined as a 5× increase in tumor volume compared to the volume at the time of treatment initiation. Mice were euthanized if tumor volumes exceeded 2000 mm³ or if tumor ulceration occurred. No mice met criteria for euthanasia due to body weight loss or adverse clinical signs.

The CT26 murine colon carcinoma cell line was obtained from American Type Culture Collection (ATCC; Manassas, Va.), then screened, cultured, tested, and stored as described above. CT26 cells used in this study were free of mycoplasma. Female BALB/c mice were obtained and housed as described above. Mice were inoculated subcutaneously in the right flank with 1×10⁵ CT26 cells in 100 μl of HBSS/Matrigel mixture. When tumors reached a volume of 130-230 mm³ (approximately 8 days after inoculation), animals were distributed into treatment groups such that variance in tumor sizes between treatment groups was minimized. Mice were treated with antibodies as described for the EMT6 model, euthanized after 10-12 days (after 3 doses of treatment), and tumors collected for flow cytometry analysis.

Sample size in the mouse studies is based on the number of mice routinely needed to establish statistical significance based on variability within study arms. Treatment arms were not blinded. All animal studies herein were approved by the Genentech Institutional Animal Care and Use Committee.

Preparation of Single-Cell Suspensions and Antibody Staining for Flow Cytometry:

Tumors were weighed, minced, and enzymatically digested using a cocktail of dispase (Life Technologies, Carlsbad, Calif.), collagenase P, and DNaseI (Roche, Penzberg, Germany) for 45 minutes at 37° C. to obtain a single-cell suspension. Cells were counted using a Vi-CELL XR (Beckman Coulter, Brea, Calif.). Draining lymph nodes were similarly minced and digested with the same cocktail for 30 minutes. All cell suspensions were passed through 100 μm pore filters to remove cell clumps and debris. For analysis of cytokine expression, cells were re-stimulated ex vivo for 3 hours at 37° C. in T cell stimulation media composed as follows: RPMI 1640 medium with 10% FBS (Hyclone, Waltham, Mass.), 100 U/ml penicillin/100 μg/ml streptomycin (Gibco, Thermo Fisher Scientific, Waltham, Mass.), 55 μM β-mercaptoethanol (Gibco, Thermo Fisher Scientific, Waltham, Mass.), 2 mM L-glutamine (Gibco, Thermo Fisher Scientific, Waltham, Mass.), 1 mM sodium pyruvate (Gibco, Thermo Fisher Scientific, Waltham, Mass.), 0.1 mM non-essential amino acids (Gibco, Thermo Fisher Scientific, Waltham, Mass.), 10 mM HEPES (Gibco, Thermo Fisher Scientific, Waltham, Mass.), and 1× Cell Stimulation Cocktail with protein transport inhibitors (containing phorbol 12-myristate 13-acetate (PMA), ionomycin, brefeldin A, and monensin; eBioscience, Thermo Fisher Scientific, Waltham, Mass.). For cell staining, cells were first incubated with anti-CD16/CD32 Fc block (5 μg/ml; BD Biosciences, San Jose, Calif.; clone 2.4G2) and LIVE/DEAD Fixable dead cell stain (APC-efluor780; Invitrogen, Carlsbad, Calif.) in PBS for 20 minutes at 4-8° C. Cells were then washed and stained with combinations of the following antibodies: CD45-BV510 (2 μg/ml; BD Biosciences, San Jose, Calif.; clone 30-F11), Thy1.2-efluor450 (2 μg/ml; eBioscience, Thermo Fisher Scientific, Waltham, Mass.; clone 53-2.1), Thy1.2-alexafluor700 (5 μg/ml; BioLegend, San Diego, Calif.; clone 53-2.1), Thy1.1-alexafluor488 (2.5 μg/ml; BioLegend, San Diego, Calif.; clone OX-7), CD4-BUV395 (2 μg/ml; BD Biosciences, San Jose, Calif., clone GK1.5), CD8a-BB515 (2 μg/ml; BD Biosciences, San Jose, Calif., clone 53-6.7), CD8a-PE (2 μg/ml; BioLegend, San Diego, Calif.; clone 53-6.7), CD8a-BUV737 (2 μg/ml; BD Biosciences, San Jose, Calif.; clone 53-6.7), CD11b-alexafluor700 (5 μg/ml; BioLegend, San Diego, Calif.; clone M1/70), Gr1-PE-Cy5.5 (1 μg/ml; eBioscience, Thermo Fisher Scientific, Waltham, Mass.; clone RB6-8C5), CD11c-PE/Dazzle594 (2 μg/ml; BioLegend, San Diego, Calif.; clone N418), MHCII (1-A/I-E)-FITC (2.5 μg/ml; eBioscience, Thermo Fisher Scientific, Waltham, Mass.; clone M5/114.15.2), CD64-PE/Cy7 (2 μg/ml; BioLegend, San Diego, Calif.; clone X₅₄-5/7.1), CD169-PE/Cy7 (2 μg/ml; BioLegend, San Diego, Calif.; clone 3D6.112), and B220-BUV737 (2 μg/ml; BD Biosciences, San Jose, Calif.; clone RA3-6B2). Cells were stained for 20 minutes at 4-8° C.

For analysis of intracellular proteins, surface-stained cells were fixed and permeabilized with the eBioscience Foxp3/Transcription Factor staining buffer set (Thermo Fisher Scientific, Waltham, Mass.). Cells were then stained for 30-60 minutes at 4-8° C. with combinations of the following antibodies in permeabilization buffer: Foxp3-efluor450 (2 μg/ml; eBioscience, Thermo Fisher Scientific, Waltham, Mass.; clone FJK-16s), Foxp3-APC (2 μg/ml; eBioscience, Thermo Fisher Scientific, Waltham, Mass.; clone FJK-16s), GzmB-Pacific Blue (1 μg/ml; BioLegend, San Diego, Calif.; clone GB11), TNF-PE (1 μg/ml; BioLegend, San Diego, Calif.; clone MP6-XT22), IFNγ-PE/Dazzle594 (0.67 μg/ml; BioLegend, San Diego, Calif.; clone XMG1.2), and IL-17A-BV786 (1 μg/ml; BioLegend, San Diego, Calif.; clone TC11-18H10).

Flow cytometry data were collected with a BD LSRFortessa or BD FACSymphony analyzer (BD Biosciences, San Jose, Calif.) and analyzed using FlowJo software (Version 10.5, FlowJo LLC, Ashland, Oreg.).

In Vivo T Cell Priming:

C57BL/6J.OT-I.Thy1.1 TCR transgenic mice were bred and housed at Genentech under specific pathogen free (SPF) conditions. Wild type C57BL/6J mice were obtained from the Jackson Laboratory (Sacramento, Calif.). Naïve OT-I T cells were isolated from spleens and lymph nodes of C57BL/6J.OT-I.Thy1.1 mice by first mashing through 70 μm pore filters using the sterile blunt end of a plunger from a 1 ml syringe. Naïve CD8⁺ T cells were then isolated using the EasySep Mouse Naïve CD8+ T cell Isolation Kit (STEMCELL Technologies, Cambridge, Mass.). Cells were resuspended at 1×10⁷ cells/ml in sterile HBSS and 1×10⁶ cells (0.1 ml) were injected intravenously via the lateral tail vein into wild type C57BL/6J recipient mice. Mice were then treated with isotype control antibodies, anti-PD-L1 (mouse IgG1 clone 6E11, 10 mg/kg first dose followed by 5 mg/kg thereafter), anti-IL6R (mouse IgG2a clone MR16-1, 15 mg/kg), or a combination of anti-PD-L1 and anti-IL6R via intraperitoneal injection. The next day mice were injected intravenously with a mixture of 50 μg/kg DEC-OVA (ovalbumin fused to anti-DEC205 antibody; produced in-house) and 2.5 mg/kg anti-CD40 antibody (produced in-house; clone FGK4.5). Mice were given a second intraperitoneal dose of anti-PD-L1, anti-IL6R, or isotype control antibodies after 3 days. On day 7, mice were sacrificed, splenocytes were isolated as described above, viable cells were counted using a Vi-CELL XR (Beckman Coulter, Brea, Calif.), and cells were stimulated for 3 hours with 1× Cell Stimulation Cocktail with protein transport inhibitors (containing phorbol 12-myristate 13-acetate (PMA), ionomycin, brefeldin A, and monensin; eBioscience, Thermo Fisher Scientific, Waltham, Mass.), as described in the preceding section. Cells were then prepared for flow cytometry analysis as described above.

Analysis of T Cell Activation Ex Vivo:

C57BL/6J.Il6r^{-/-}, C57BL/6J.OT-1 TCR transgenic, and C57BL/6J.Stat3^{wt/loxp} Cd4.cre⁺ mice were bred and housed at Genentech under specific pathogen free (SPF) conditions. Wild type C57BL/6J mice were obtained from the Jackson Laboratory (Sacramento, Calif.). Mouse spleens and/or lymph nodes were isolated and mashed through 70 μm pore filters using the sterile blunt end of a plunger from a 1 ml syringe. For peptide activation of OT-1CD8⁺ T cells, 0.2 million splenocytes were seeded in Falcon flat bottom 96 well plates (Corning Life Sciences, Corning, N.Y.) in T cell media as described above (minus cell stimulation cocktail). Cells were pulsed with 100 ng/ml SIINFEKL peptide (SEQ ID NO:34) (AnaSpec, Fremont, Calif.). After 2 days, cells were analyzed or transitioned to T cell media (without SIINFEKL) containing 10 ng/ml recombinant human IL-2 and incubated for a further 3 days before use in cytotoxicity assays or re-stimulation with anti-CD3 and anti-CD28 antibodies and flow cytometry analysis. For flow cytometry analysis of cytokine expression, 1× protein transport inhibitor cocktail (containing brefeldin A and monensin; eBioscience, Thermo Fisher Scientific, Waltham, Mass.) was added 4 hours before staining.

For polyclonal T cell activation, bulk splenocytes or CD8⁺ T cells isolated using the EasySep CD8⁺ T cell Isolation Kit (STEMCELL Technologies, Cambridge, Mass.) were plated in T cell media at 0.2 million cells per well in Falcon flat bottom 96 well plates (Corning Life Sciences, Corning, N.Y.) that had been coated overnight with 5 μg/ml anti-CD3 antibody (BD Biosciences, San Jose, Calif., clone 145-2C111). Anti-CD28 antibody was added to culture medium at a concentration of 2.5 μg/ml (BD Biosciences, San Jose, Calif., clone 37.51). Unless specified otherwise, recombinant human IL-2 (R&D Systems, Minneapolis, Minn.) was added to cultures at 10 ng/ml to promote T cell viability and expansion. To facilitate tracking of cell division, cells were labelled in some experiments with Cell Trace Violet-421 (Molecular Probes, Thermo Fisher Scientific, Waltham, Mass.) according to manufacturer instructions prior to plating. In some assays, T cells were activated in the presence of recombinant mouse IL-6 (10 ng/ml; R&D Systems, Minneapolis, Minn.), mouse hyper-IL-6 (20 ng/ml; R&D Systems, Minneapolis, Minn.), mouse IL-15/1L-15Ra complex (10 ng/ml; eBioscience, Thermo Fisher Scientific, Waltham, Mass.), isotype control mouse IgG2a antibody (5 μg/ml), or mouse IgG2a anti-IL6R antibody (5 μg/ml; clone MR16-1).

For flow cytometry analysis, cells were first incubated with Fc blocking reagent and viability dye (APC-efluor780) as described for flow cytometry analysis of tumor and lymph node-derived cells. Cells were then surface stained for 20 minutes at 4-8° C. with the following antibodies: CD8a-BB515 (2 μg/ml; BD Biosciences, San Jose, Calif., clone 53-6.7) and CD4-BUV395 (2 μg/ml; BD Biosciences, San Jose, Calif., clone GK1.5). Cells were fixed and permeabilized with the eBioscience Foxp3/Transcription Factor staining buffer set (Thermo Fisher Scientific, Waltham, Mass.) and stained for 30-60 minutes at 4-8° C. with the following antibodies in permeabilization buffer: TNF-PE (1 μg/ml; BioLegend, San Diego, Calif.; clone MP6-XT22), IFNγ-PE/Dazzle594 (0.67 μg/ml; BioLegend, San Diego, Calif.; clone XMG1.2), and GzmB-efluor660 (1 μg/ml; eBioscience, Thermo Fisher Scientific, Waltham, Mass.; clone NGZB). Flow cytometry data were collected with a BD LSRFortessa or BD FACSymphony analyzer (BD Biosciences, San Jose, Calif.) and analyzed using FlowJo software (Version 10.5, FlowJo LLC, Ashland, Oreg.).

T Cell Cytotoxicity Assays:

OT-I CD8⁺ T cells were activated with SIINFEKL peptide (SEQ ID NO:34) (AnaSpec, Fremont, Calif.) as described above in the presence or absence of recombinant mouse IL-6 (10 ng/ml) or recombinant mouse hyper-IL-6 (20 ng/ml). Cells were used in cytotoxicity assays after 5-6 days. One day prior to starting the cytotoxicity assay, MC38-GFP or MC38-GFP-OVA cells (engineered to express ovalbumin) were plated in Falcon flat-bottom 96-well plates (Corning Life Sciences, Corning, N.Y.) at 5,000 cells per well. Parental MC38 cells were originally acquired from ATCC (Manassas, Va.). Cells were characterized and maintained as described for EMT6 cells, and were free of mycoplasma contamination. MC38-GFP cells were then pulsed with 10 ng/ml SIINFEKL peptide (SEQ ID NO:34) for 1 hour at 37° C., washed with PBS, and activated T cells added in complete T cell medium at various ratios (0:1, 1:1, 5:1, 10:1, or 20:1). For killing of MC38-GFP-OVA cells, T cells were added directly without additional SIINFEKL peptide (SEQ ID NO:34) to test killing in the setting of endogenous antigen presentation. MC38 cell killing was quantified over time using IncuCyte Live Cell Analysis (Essen Bioscience, Ann Arbor, Mich.). Data were collected from the phase contrast and GFP channels using the 10× objective. GFP+ area (which is directly proportional to the number of viable MC38-GFP cells) was quantified every hour and normalized to matched timepoints of MC38 cells cultured in the absence of T cells.

Bulk RNA-Sequencing Analysis of T Cells:

OT-I CD8⁺ T cells were activated with SIINFEKL peptide (SEQ ID NO:34) as described above (see Analysis of T cell activation ex vivo). Experimental treatment conditions were as follows: (1) control (no treatment); (2) recombinant mouse IL-6, 10 ng/ml; (3) recombinant mouse hyper-IL-6, 20 ng/ml; (4) mouse IgG2a isotype control antibody, 5 μg/ml; and (5) mouse IgG2a anti-IL6R antibody (clone MR16-1), 5 μg/ml. Viable CD8⁺ T cells were sorted to >99% purity on day 7 using a BD FACS Aria Fusion cell sorter (BD Biosciences, San Jose, Calif.). Cells were then lysed in RLT buffer and RNA was extracted using the RNEasy mini kit (Qiagen, Germantown, Md.). Quality control of total RNA was done to determine sample quantity and quality. The concentration of RNA samples was determined using NanoDrop 8000 (Thermo Fisher Scientific, Waltham, Mass.) and the integrity of RNA was determined by Fragment Analyzer (Agilent Technologies, Santa Clara, Calif.). 0.1 μg of total RNA was used as an input material for library preparation using TruSeq Stranded Total RNA Library Prep Kit (Illumina, San Diego, Calif.). Size of the libraries was confirmed using 4200 TapeStation and High Sensitivity D1 K screen tape (Agilent Technologies, Santa Clare, Calif.) and their concentration was determined by a quantitative PCR-based method using Library quantification kit (KAPA Biosystems, Wilmington, Mass.). The libraries were multiplexed and sequenced on Illumina HiSeq4000 (Illumina, San Diego, Calif.) to generate 30 million single-end 50 base pair reads.

RNA-sequencing data were analyzed using HTSeqGenie (Reeder & Pau, G. HTSeqGenie: a NGS analysis pipeline. R package version 3.14.0 (2012) in BioConductor (Huber et al. Nat. Methods 12: 115-121 (2015) as follows: first, reads with low nucleotide qualities (70% of bases with quality <23) or rRNA and adapter contamination were removed. The reads that passed were then aligned to the reference genome GRCh38.p10 using GSNAP (Wu et al. (2010), supra). Alignments of the reads that were reported by GSNAP as “uniquely mapping” were used for subsequent analysis. Gene expression levels were quantified as Reads Per Kilobase of exon model per Million mapped reads normalized by size factor (nRPKM), defined as number of reads aligning to a gene in a sample/(total number of uniquely mapped reads for that sample×gene length×size factor). Principal components analysis (PCA) was performed using Partek Flow version 8.0.19.0710. Differential gene expression analysis was performed using voom from the limma R package (Ritchie et al. Nucleic Acids Res. 43: e47-e47 (2015)).

Statistical Analysis of Pre-Clinical Data:

Statistical analyses and production of graphs was performed using Prism 7 (Graphpad Software, San Diego, Calif.). The specific statistical tests (and multiple testing correction methods) used are indicated in figure legends. P-values and FDR values <0.05 were considered in all analyses to be statistically significant.

### Example 3: Plasma IL-6 is Associated with Poor Clinical Outcome to Atezolizumab (Anti-PD-L1) and Reduced CD8⁺ T Cell Activation in Cancer Patients

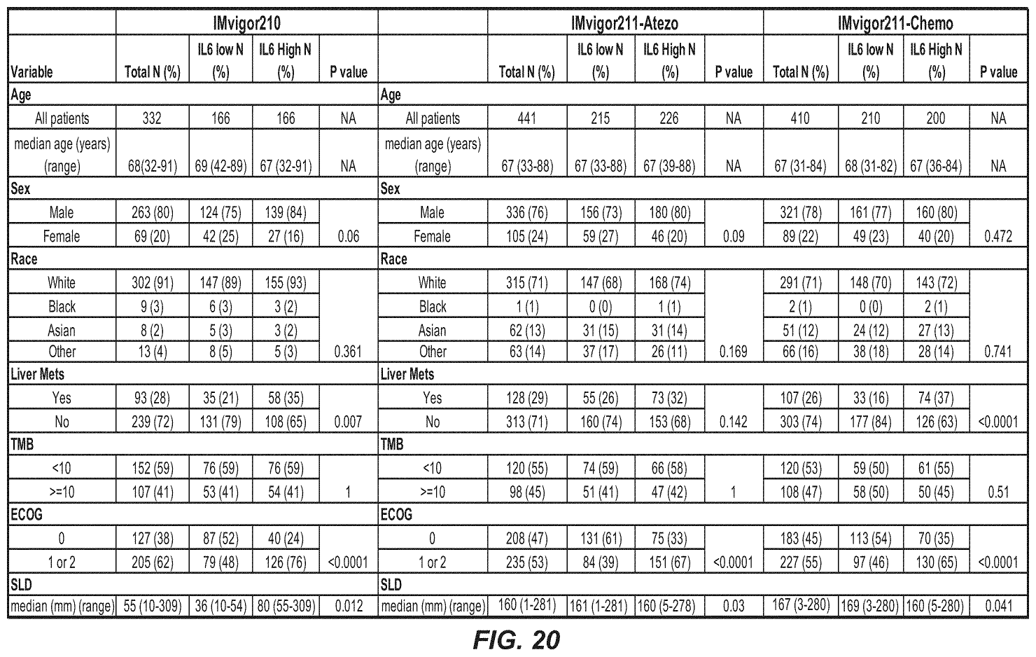

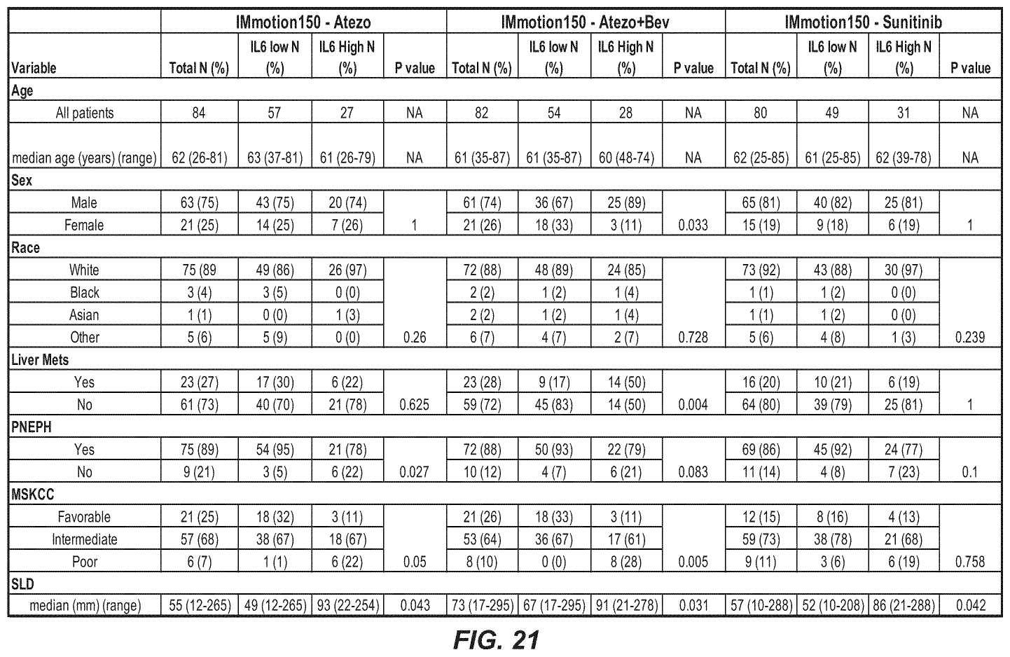

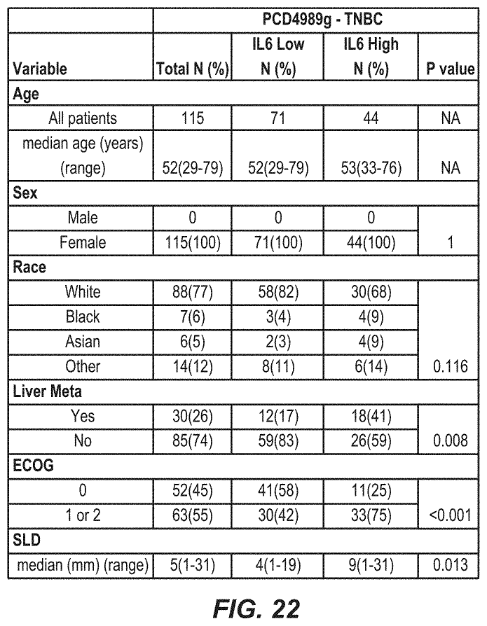

The association between plasma IL-6 and outcomes in clinical trials of atezolizumab in patients with metastatic triple negative breast cancer (mTNBC), metastatic renal cell carcinoma (mRCC), and metastatic urothelial bladder carcinoma (mUC) was evaluated (FIG. 5). PCD4989g was a single-arm Phase I study that evaluated atezolizumab in patients with locally advanced or metastatic malignancies, including mTNBC (Emens et al. JAMA Oncol 5: 74-82 (2019)). IMvigor210 was a single-arm Phase II study of atezolizumab in mUC (Rosenberg et al. Atezolizumab in patients with locally advanced and metastatic urothelial carcinoma who have progressed following treatment with platinum-based chemotherapy: a single-arm, multicentre, phase 2 trial. Lancet 387: 1909-1920 (2016); Balar et al. Lancet 389: 67-76 (2017)). IMvigor211 was a randomized Phase III mUC trial in which patients with prior platinum-based chemotherapy were treated with either chemotherapy (taxanes or vinflunine) or atezolizumab (Powles et al. Lancet 391: 748-757 (2018)). IMmotion150 was a randomized Phase II trial that investigated atezolizumab with or without bevacizumab versus the anti-angiogenic tyrosine kinase inhibitor sunitinib in patients with untreated mRCC (McDermott et al. Nature Med 24: 749-757 (2018)). Demographic characteristics of biomarker-evaluable patients with high or low levels of plasma IL-6 are presented in FIGS. 20-23. Multivariate analyses (co-variates defined in figure legends and Methods) were conducted to identify associations with clinical outcomes, reported here as adjusted hazard ratios (HR).

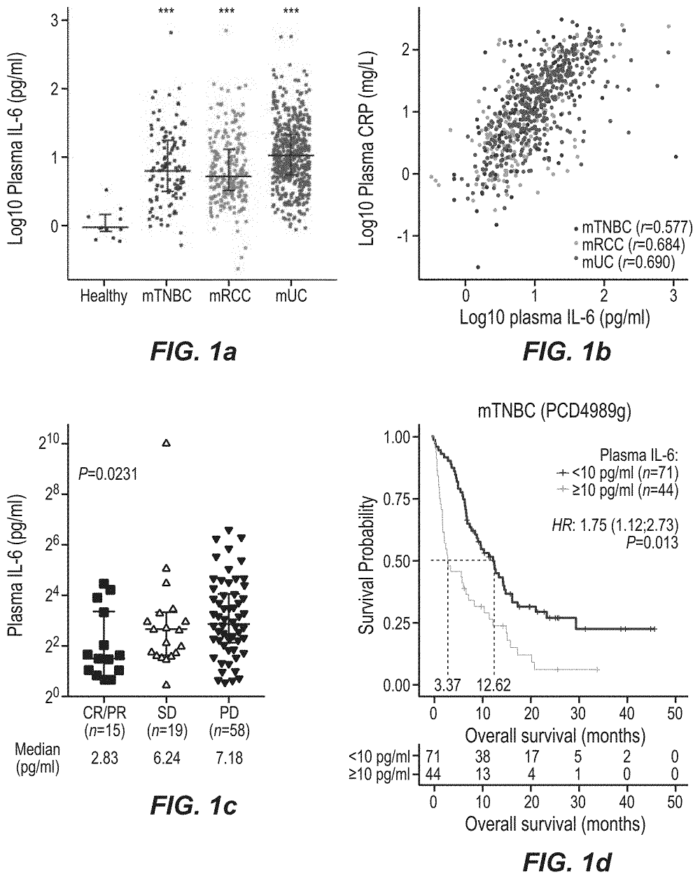

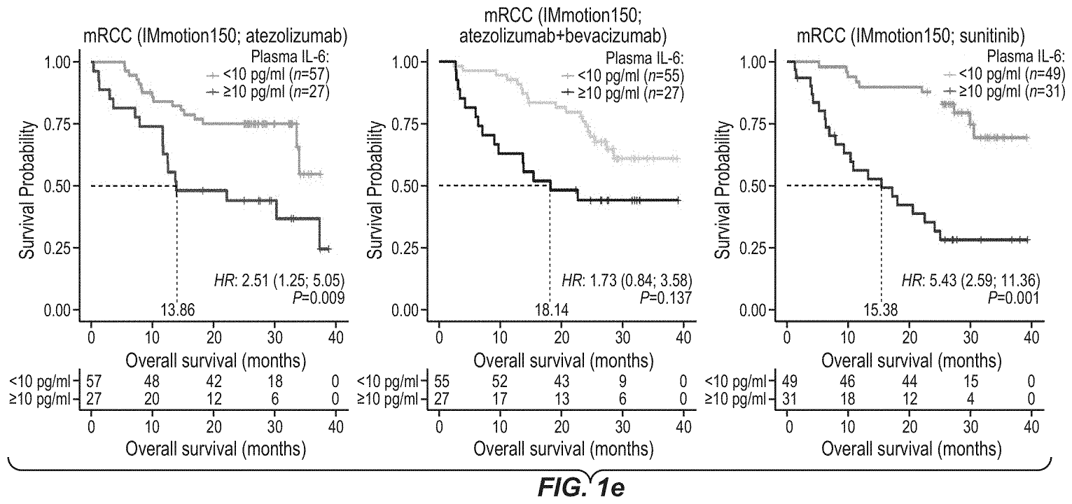

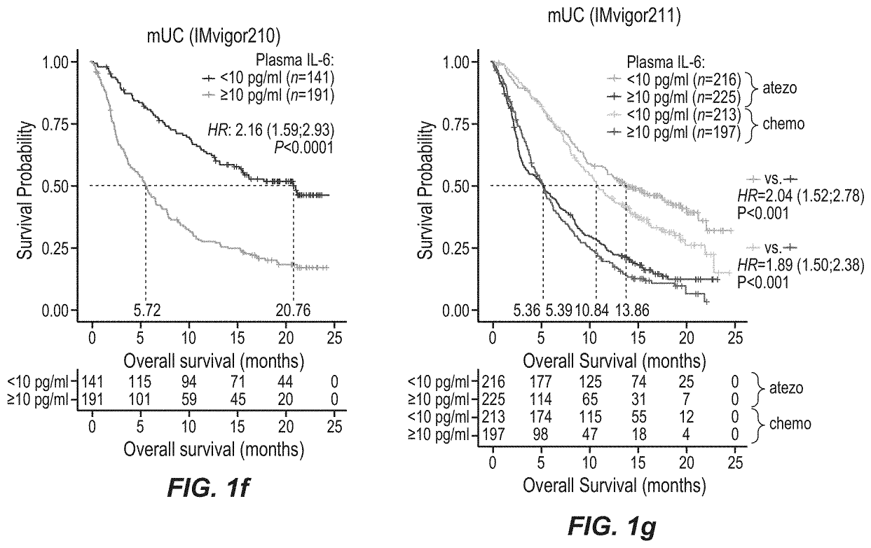

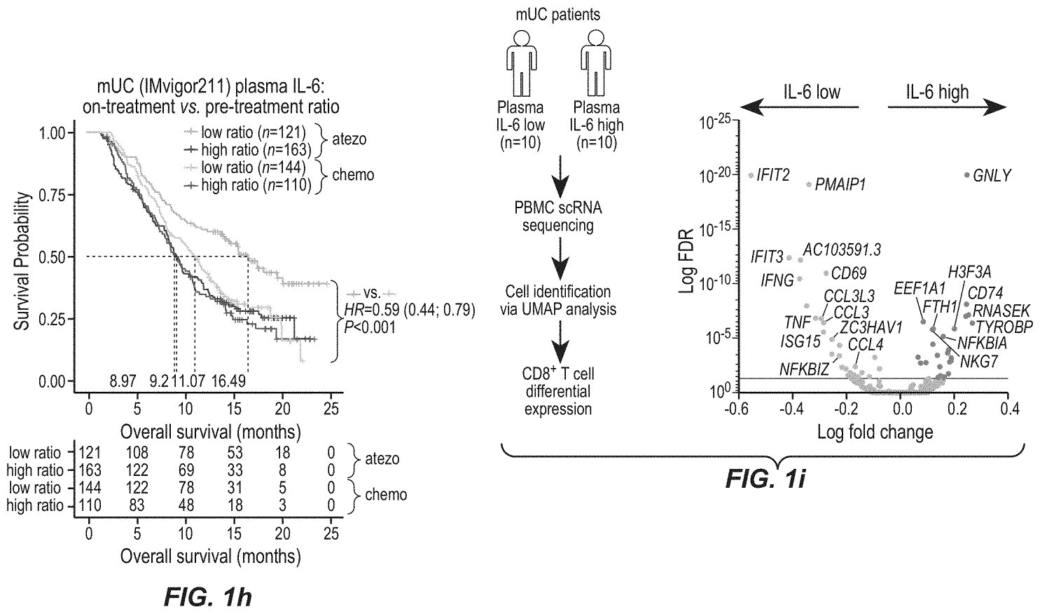

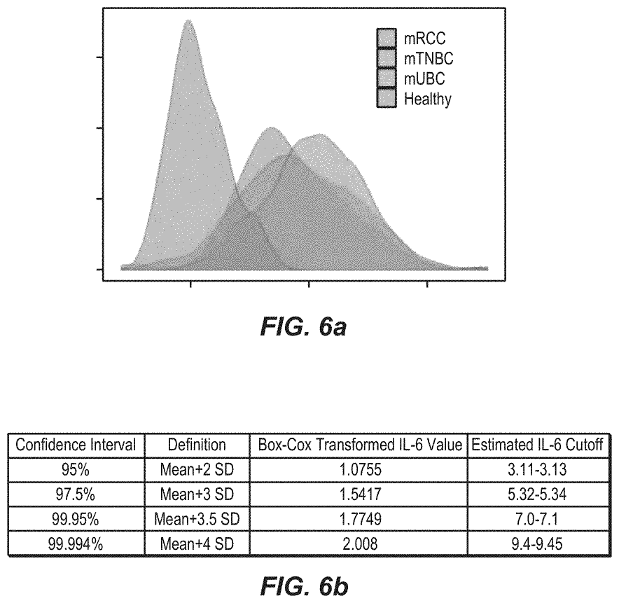

Compared to healthy adults, plasma IL-6 levels were significantly higher in patients with mTNBC, mRCC, or mUC (FIG. 1a) and correlated closely with plasma CRP, an IL-6-inducible biomarker of systemic inflammation (FIG. 1b). Based on the distribution of plasma IL-6 in our datasets, we defined high IL-6 concentration as ≥10 μg/ml (4 standard deviations above the mean concentration in healthy adults; see Example 1 and FIGS. 6a-b).

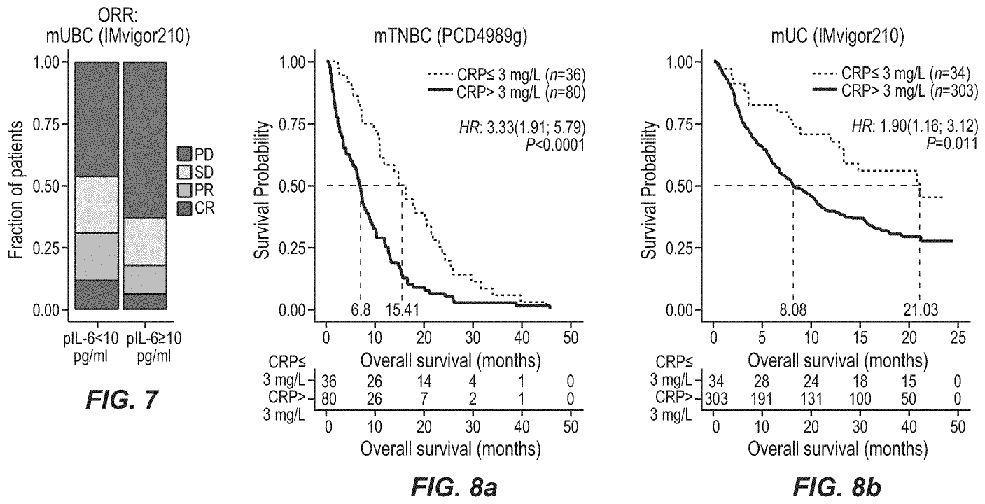

In PCD4989g (mTNBC), patients who showed objective response following atezolizumab treatment had lower baseline plasma IL-6 compared to patients with stable or progressive disease (P=0.0231, FIG. 1c). Moreover, high baseline plasma IL-6 was associated with reduced overall survival (OS) (HR: 1.75, 95% CI: 1.12, 2.73; P=0.013) (FIG. 1d). In IMmotion150 (mRCC), elevated plasma IL-6 was associated with reduced OS in patients treated with atezolizumab (HR: 2.51, 95% CI: 1.25, 5.05; P=0.014) or sunitinib (HR: 5.43, 95% CI: 2.59, 11.36; P<0.001; FIG. 1e). In patients treated with atezolizumab plus bevacizumab, high plasma IL-6 was also associated with poor OS, but this did not reach statistical significance after adjustment for baseline prognostic factors (FIG. 1e). In IMvigor210 (mUC), high plasma IL-6 was associated with poor OS (HR: 2.16, 95% CI: 1.59, 2.93; P<0.0001) and reduced objective response rates (FIG. 1f, FIG. 7). In the randomized IMvigor211 trial (mUC), patients with high plasma IL-6 had significantly worse OS in both the atezolizumab (HR: 2.42, 95% CI: 1.92, 3.07; P<0.001) and chemotherapy arms (HR: 1.89, 95% CI: 1.50, 2.38, P<0.001) (FIG. 1g), indicating that high plasma IL-6 is associated with worse prognosis in mUC. While atezolizumab improved OS compared to chemotherapy among patients with low plasma IL-6 (HR: 0.73, 95% CI: 0.57, 0.93; P=0.012), an interaction test for treatment arm effect showed that this difference was not statistically significant.

An association between high plasma CRP and reduced OS was also observed in these trials (FIG. 8a-c), consistent with the strong correlation between CRP and plasma IL-6 (FIG. 1b).

To evaluate the clinical correlates of changes in plasma IL-6 during treatment, we assessed plasma IL-6 levels after 6 weeks of therapy and compared them to matched baseline values in the IMvigor211 trial. Notably, an on-treatment increase in plasma IL-6 was associated with reduced OS in patients treated with atezolizumab (HR: 2.04, 95% CI: 1.52-2.78, P<0.001) but not with chemotherapy (HR: 1.26, 95% CI: 0.96, 1.66, P=0.087 FIG. 1h). Compared to chemotherapy, atezolizumab improved OS in patients whose plasma IL-6 did not increase during therapy (low ratio) (HR: 0.59, 95% CI: 0.44, 0.79; P<0.001) as opposed to patients whose plasma IL-6 increased during therapy (FIG. 1h). This result shows that an on-treatment reduction in plasma IL-6 may be associated with anti-PD-L1-mediated improvement in survival.

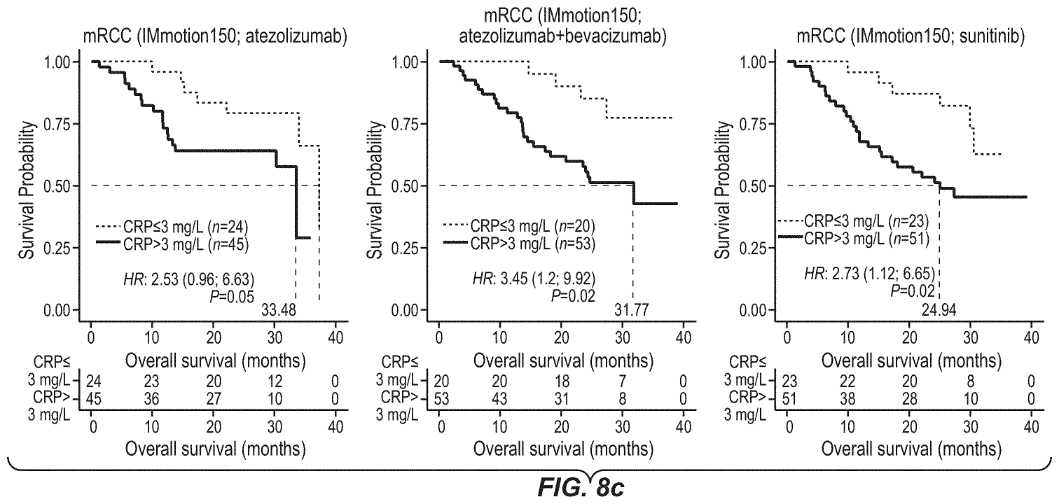

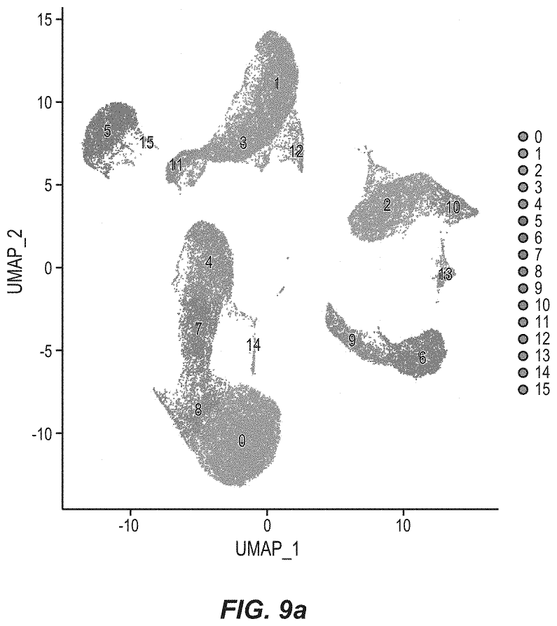

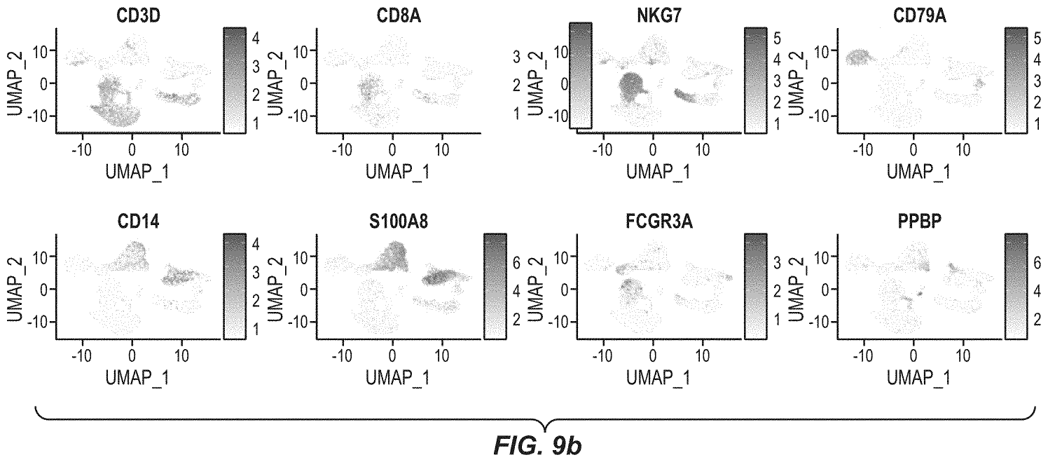

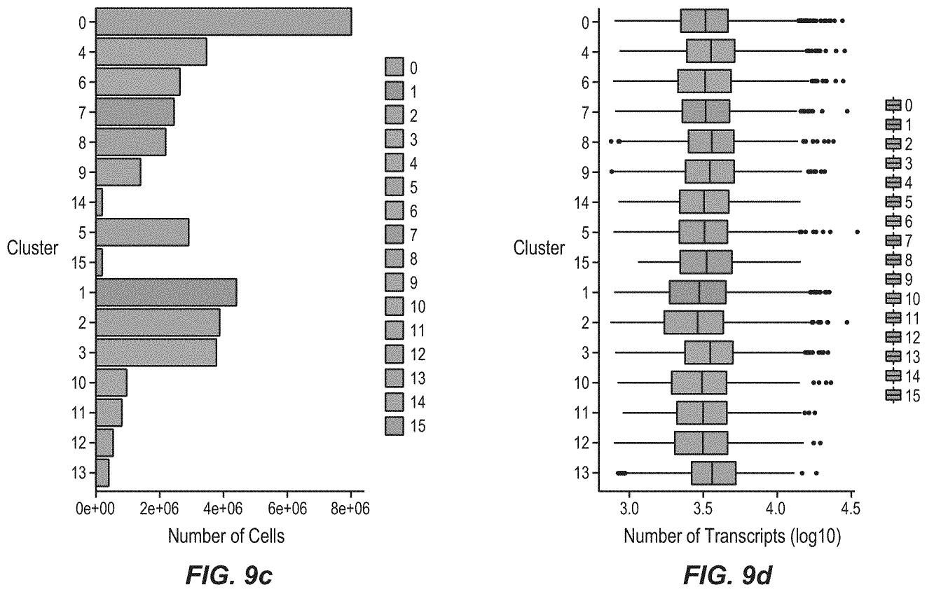

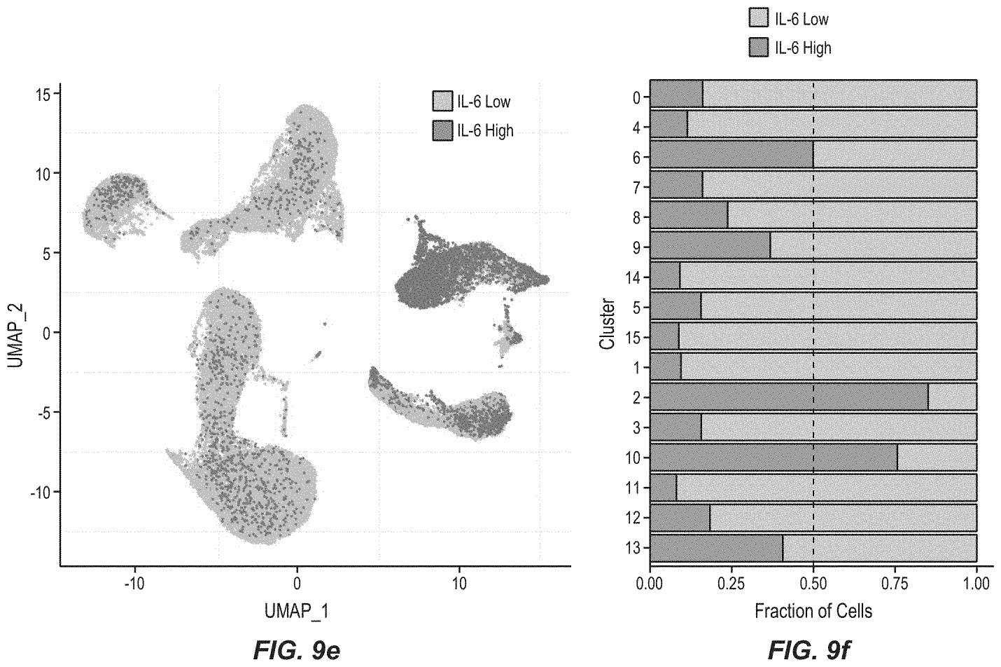

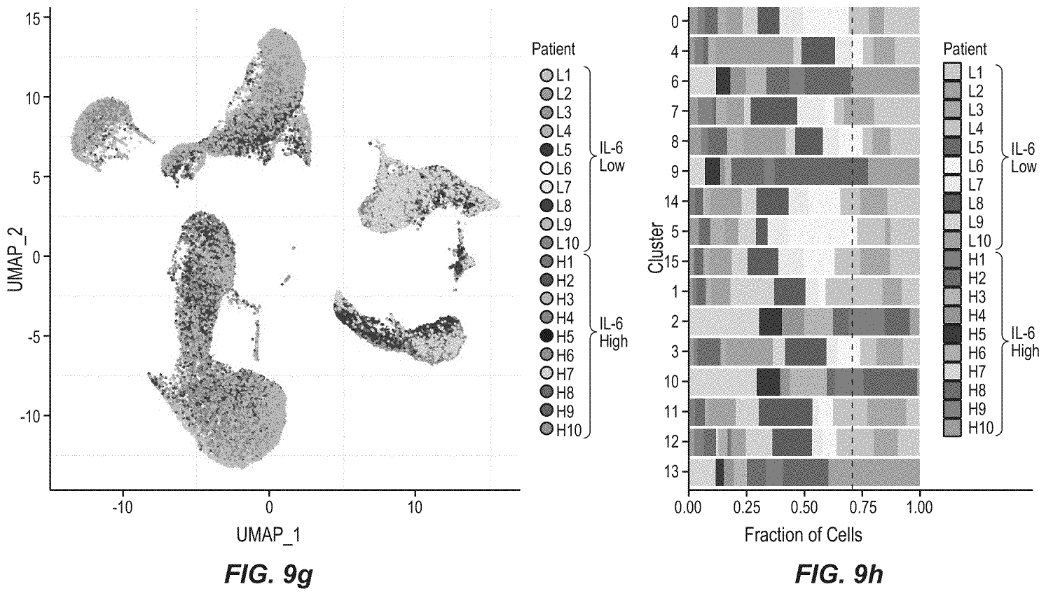

The consistent relationship between high plasma IL-6 and poor atezolizumab response raises the possibility that IL-6 contributes to immunotherapy resistance. However, the impact of IL-6 on CD8⁺ T cells, the primary effector cells of anti-tumor immunity, has not been well characterized. To determine whether plasma IL-6 is associated with altered T cell function in cancer patients, we performed single cell RNA sequencing (scRNAseq) of pre-treatment PBMCs (peripheral blood mononuclear cells) from plasma IL-6 high (n=10) or plasma IL-6 low (n=10) mUC patients from the IMvigor210 study. We identified cell subsets using Uniform Manifold Approximation and Projection (UMAP) and lineage specific genes (FIGS. 9a-h). Intriguingly, comparison of CD8⁺ T cells from plasma IL-6 low vs high patients revealed significantly higher expression of genes associated with effector function and interferon signaling (e.g. CD69, IFNG, TNF, ISG15, IFIT2, and IFIT3) in patients with low plasma IL-6. (FIG. 1i).

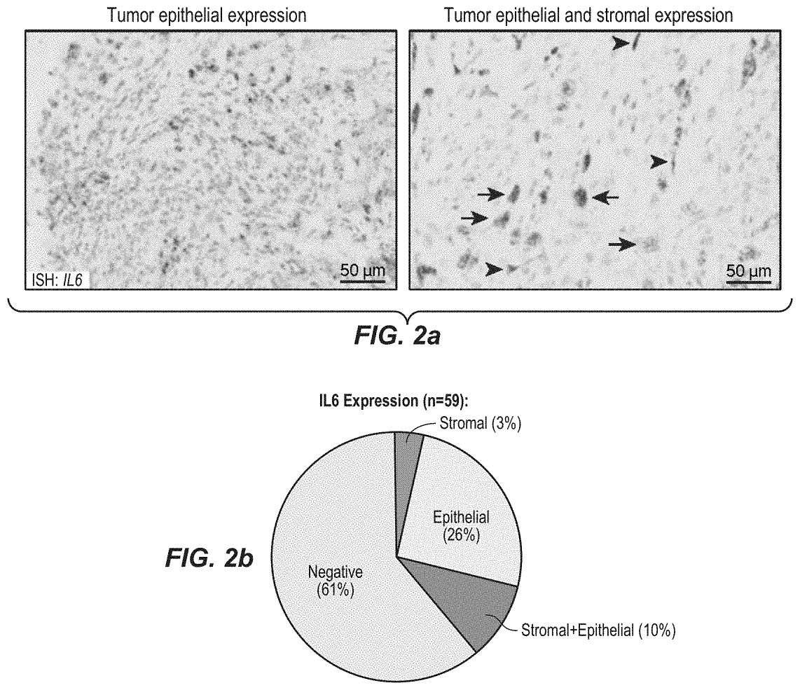

To evaluate IL-6 production in the tumor microenvironment and its effect on clinical outcomes, we conducted in situ hybridization (ISH, FIG. 2a) to assess IL6 mRNA expression in a subset of mRCC tumors (n=59) from IMmotion150. Positive IL6 expression (detected in 1% of cells) was observed in 39% of the assessed tumors, and occurred in multiple cell types, including tumor cells and stromal cells (FIG. 2b). Elevated tumor IL6 expression (as evaluated by RNASeq analysis of whole tumor tissue) was associated with reduced OS in patients treated with atezolizumab (HR: 2.72, 95% CI: 1.30, 5.72, P=0.008) (FIG. 2c). In patients treated with atezolizumab plus bevacizumab, tumor IL6 was also associated with worse OS, but this did not reach statistical significance after adjustment for baseline prognostic factors (FIG. 2c). Pre-existing anti-tumor immunity, as assessed by an intratumoral CD8⁺ T cell gene expression signature (T cell GES), was broadly associated with improved clinical outcomes to check point inhibition treatment in previous studies. Notably, high tumor IL6 expression was associated with worse OS even among patients with a high T cell GES (HR: 4.30, 95% CI: 1.35, 13.71, P=0.014, FIG. 2d).

### Example 4: IL-6 Inhibits CD8⁺ T Cell Effector Function

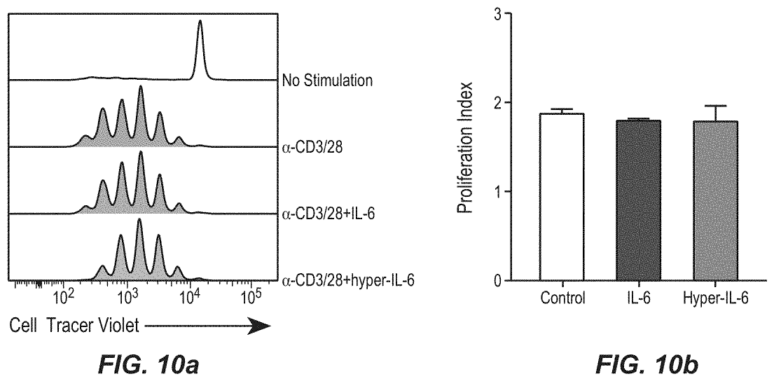

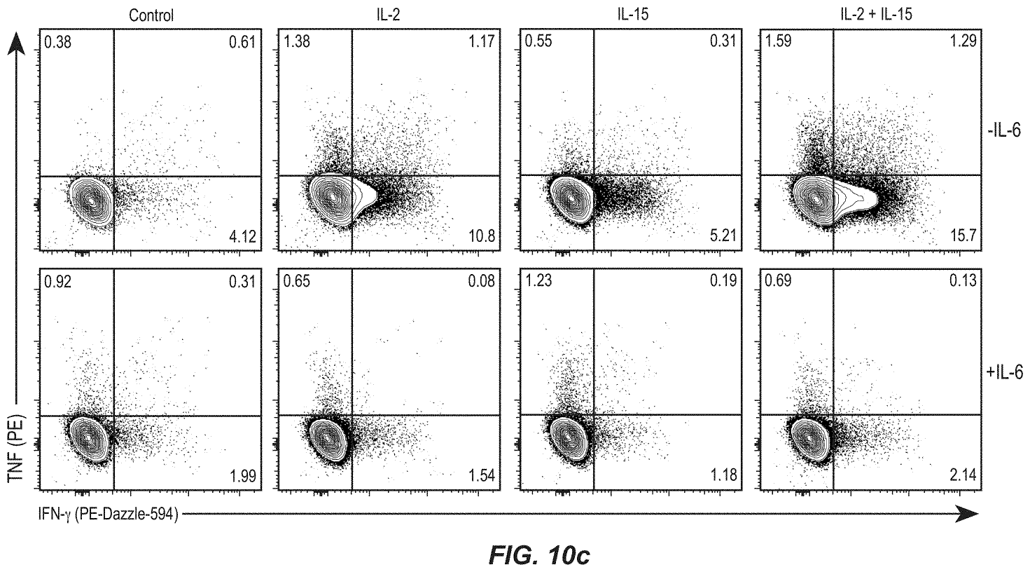

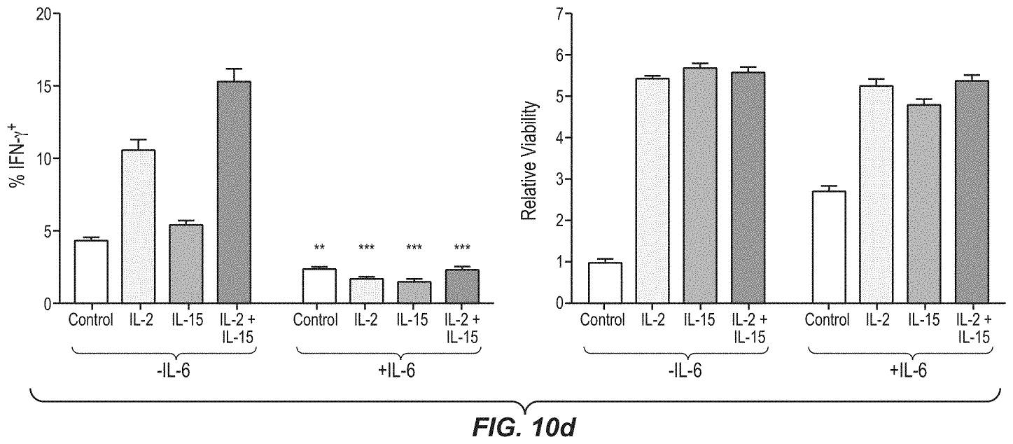

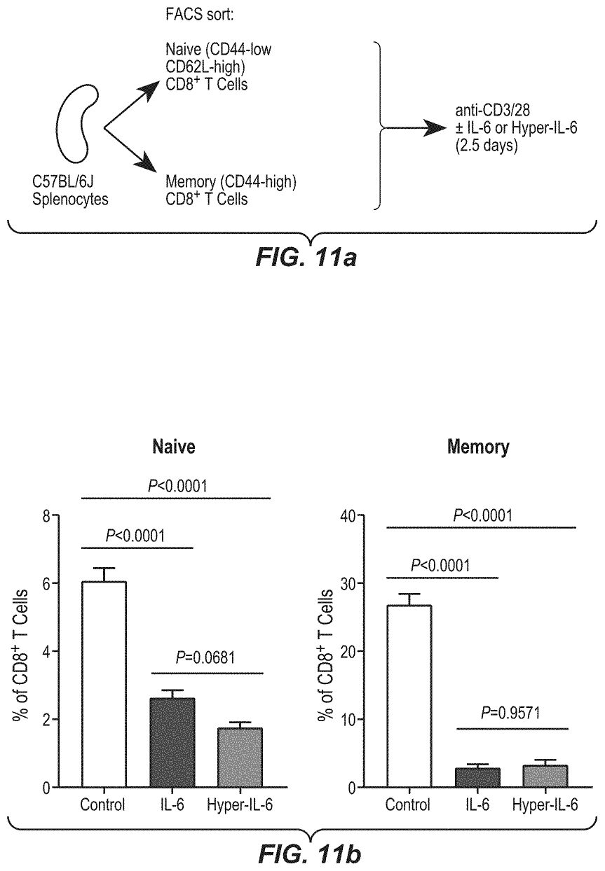

scRNAseq analysis of peripheral blood cells from mUC patients indicated that high plasma IL-6 is associated with reduced CD8⁺ T cell effector activation. (FIG. 1i). To characterize the functional impact of IL-6 on CD8⁺ T cells, we conducted a series of pre-clinical studies using animal models. Following anti-CD3/anti-CD28 stimulation of splenocytes from wild type (WT) or IL6R-deficient (Il6r^{-/-}) mice, we observed high expression of the effector molecules IFN-γ (interferon gamma), TNF (tumor necrosis factor), and GzmB (granzyme B) in Il6r^{-/-} CD8⁺ T cells and in WT cells treated with an IL6R-blocking antibody (FIG. 3a-b). In contrast, both recombinant IL-6 and hyper-IL-6 (a fusion protein of IL-6 and IL6R that elicits trans signaling through direct engagement of gp130) inhibited expression of effector molecules by WT cells while, as expected, only hyper-IL-6 did so for Il6r^{-/-} cells (FIG. 3a-b). Treatment of isolated CD8⁺ T cells with IL-6 had little effect on cell proliferation following anti-CD3/CD28 stimulation (FIGS. 10a-b), but significantly diminished cytokine expression, indicating selective regulation of effector function (FIG. 3c). While the cytokines IL-2 and IL-15 are well known to promote optimal expansion and effector differentiation of CD8⁺ T cells, IL-6 inhibited effector function with or without the addition of IL-2 or IL-15/1L-15Ra complex (FIGS. 10c-d). Importantly, IL-6-driven suppression of cytokine production was strictly dependent on the transcription factor STAT3 (signal transducer and activator of transcription 3; FIG. 3d). Notably, IL-6 repressed effector functions of both naïve and memory CD8⁺ T cells following anti-CD3/CD28 stimulation (FIG. 11a-b).

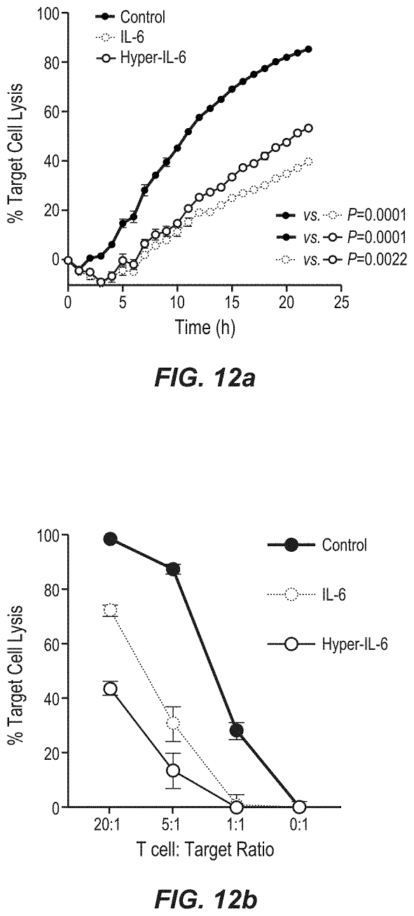

To confirm these findings in an antigen-specific setting, we incubated splenocytes from OT-I T cell receptor (TCR)-transgenic mice with SIINFEKL peptide (SEQ ID NO: 34) (a high-affinity ovalbumin epitope recognized by the OT-I TCR) and assessed their functional properties after one week. Exposure to IL-6 or hyper-IL-6 during activation caused a 5-10-fold reduction in polyfunctionality, defined as co-expression of IFN-y, TNF, and GzmB (FIG. 3e). Consistent with these data, IL-6-conditioned OT-I cells failed to efficiently kill SIINFEKL-pulsed (SEQ ID NO:34) or ovalbumin-expressing target cells, indicating impaired cytotoxicity (FIG. 3f and FIG. 12a-b).

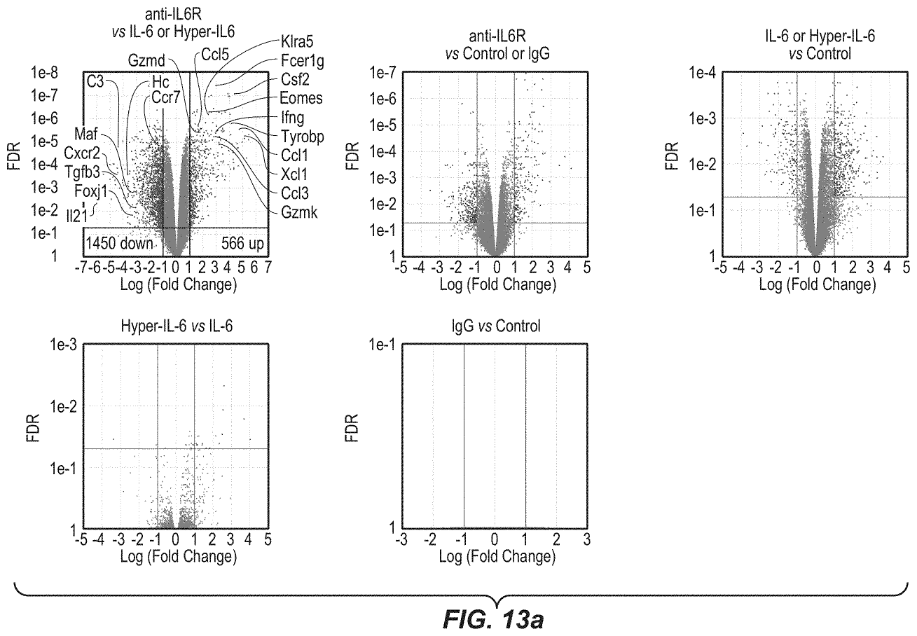

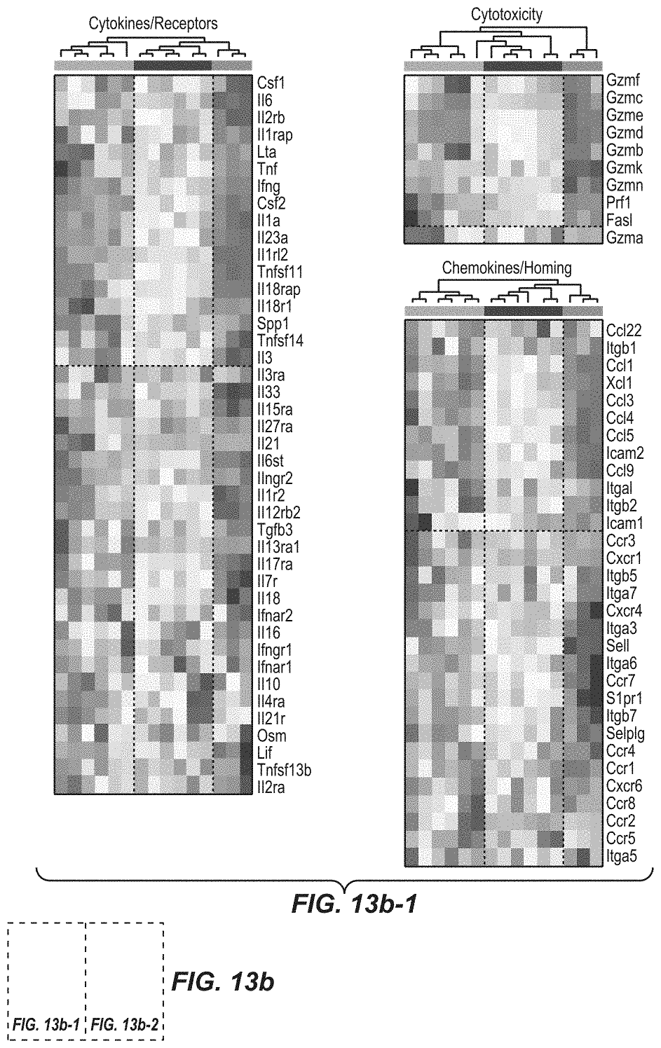

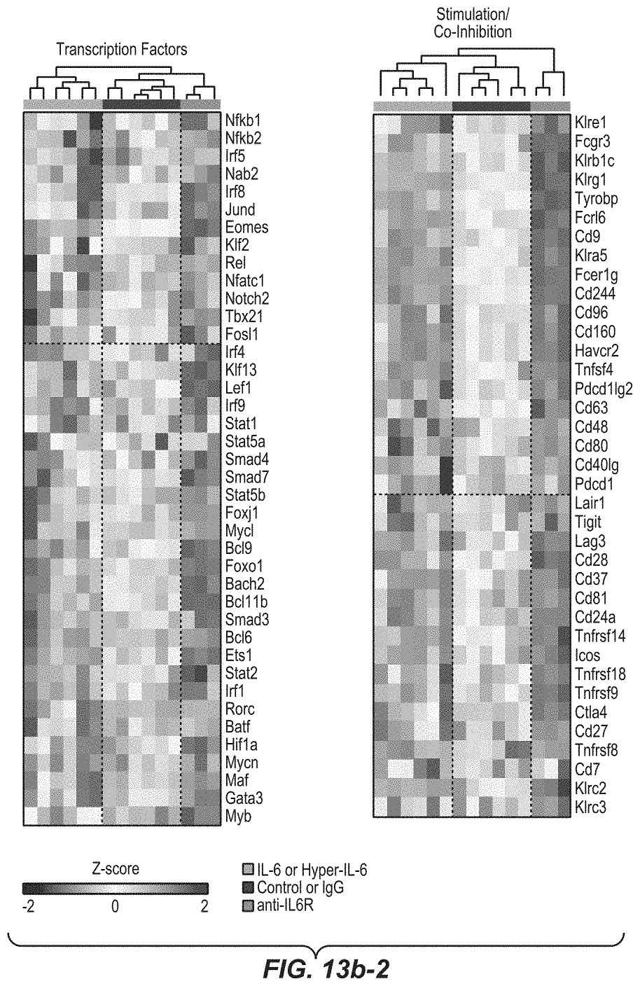

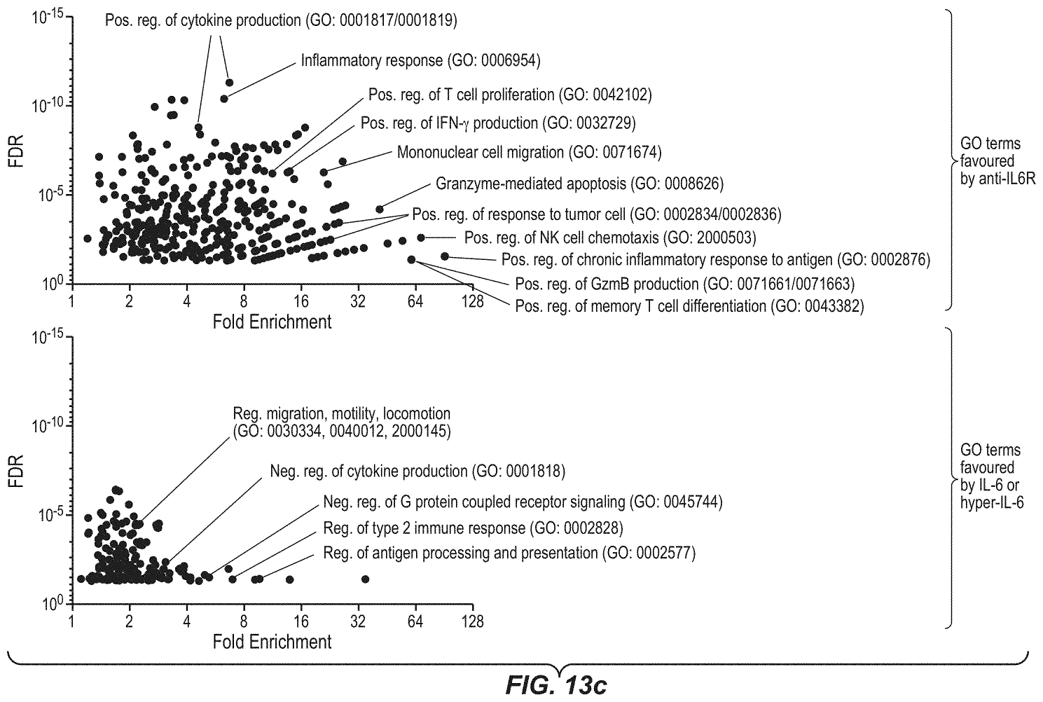

To more broadly examine the impact of IL-6 on CD8⁺ T cell function, we performed RNA-sequencing of peptide-stimulated OT-I cells. IL-6 and hyper-IL-6 drove a similar gene expression profile that was highly distinct from cells activated in the presence of IL6R blocking antibody, while control conditions (basic culture conditions or isotype control antibody) induced an intermediate phenotype (FIG. 3g, FIG. 13a, FIG. 23). Inhibition of IL-6 signaling promoted high expression of cytotoxic factors, cytokines, chemokines, and transcription factors that are critical for effector differentiation (e.g. Tbx21 and Eomes¹¹⁻¹⁴). In contrast, exogenous IL-6 promoted expression of factors that oppose T cell activation and effector differentiation (e.g. Ctla4, Foxo1, Bach2, Batt¹⁵⁻¹⁸) and genes associated with naïve or central memory cells, including Ccr7 and Sell (FIG. 3h and FIGS. 13b-1, and 13b-2). Gene ontology (GO) analysis confirmed that IL-6 blockade promoted cytotoxic effector polarization, whereas IL-6 treatment promoted repression of cytokine production (FIG. 13c and FIG. 24).

Notably, IL-6 potently suppressed the acquisition of an Eomes+Tbet+CD62L⁻ effector phenotype, while expression of the stem cell memory marker TCF1 was largely unaffected (FIG. 3i-j). Together, these data show that IL-6 signals via STAT3 to repress the acquisition of effector function by CD8⁺ T cells.

### Example 5: Combined Blockade of IL6R and PD-L1 Enhances CD8⁺ T Cell Activation and Promotes Tumor Control

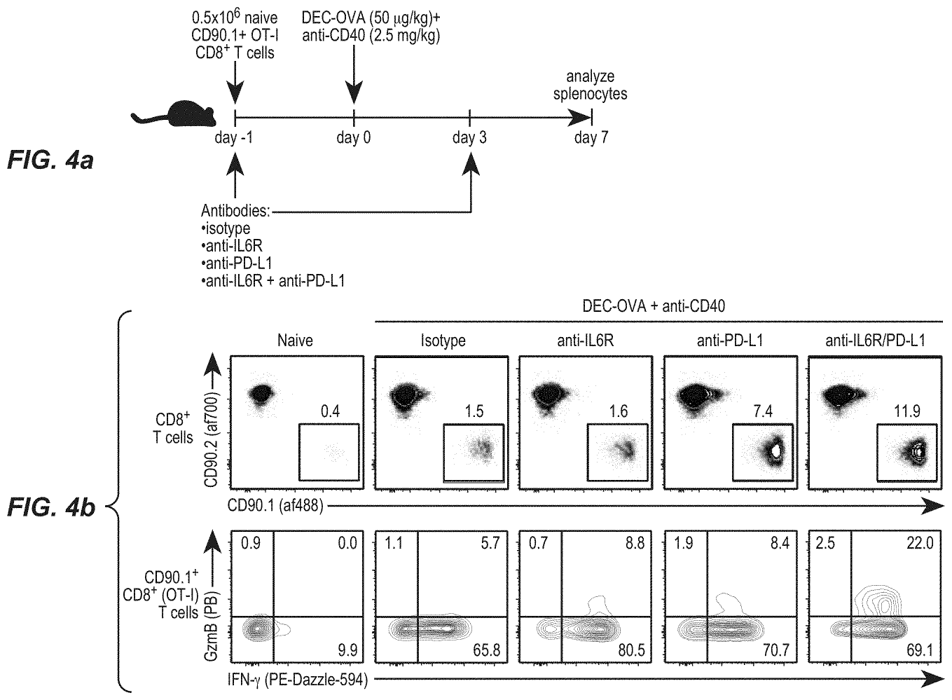

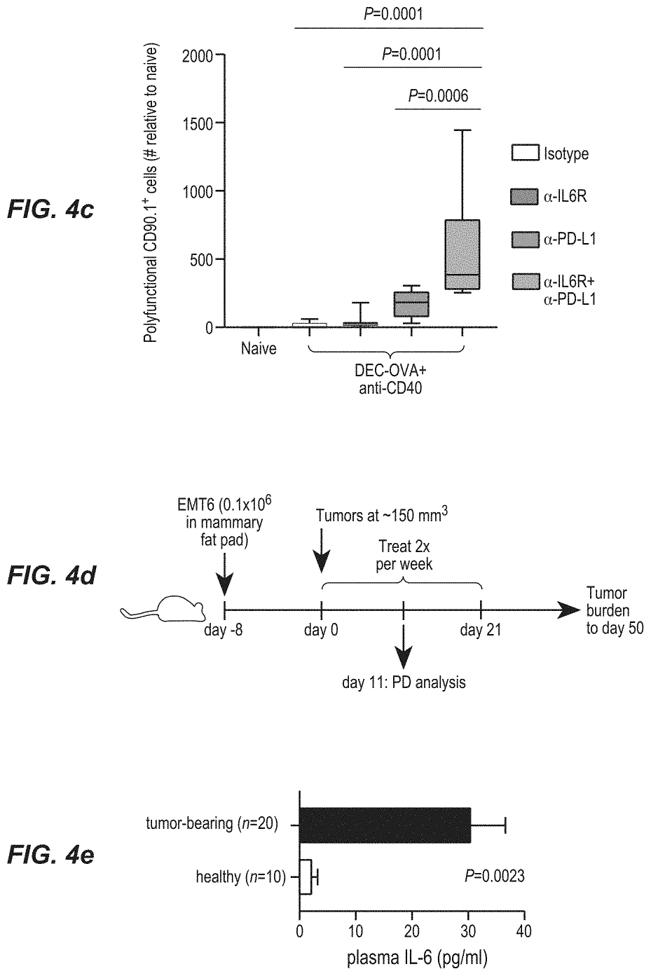

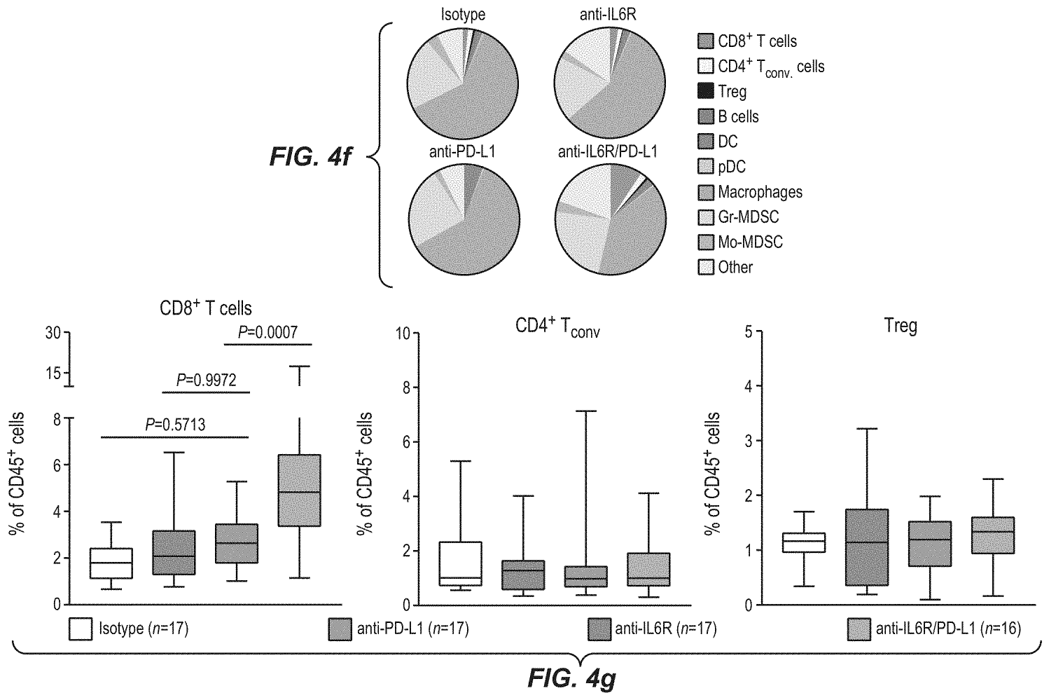

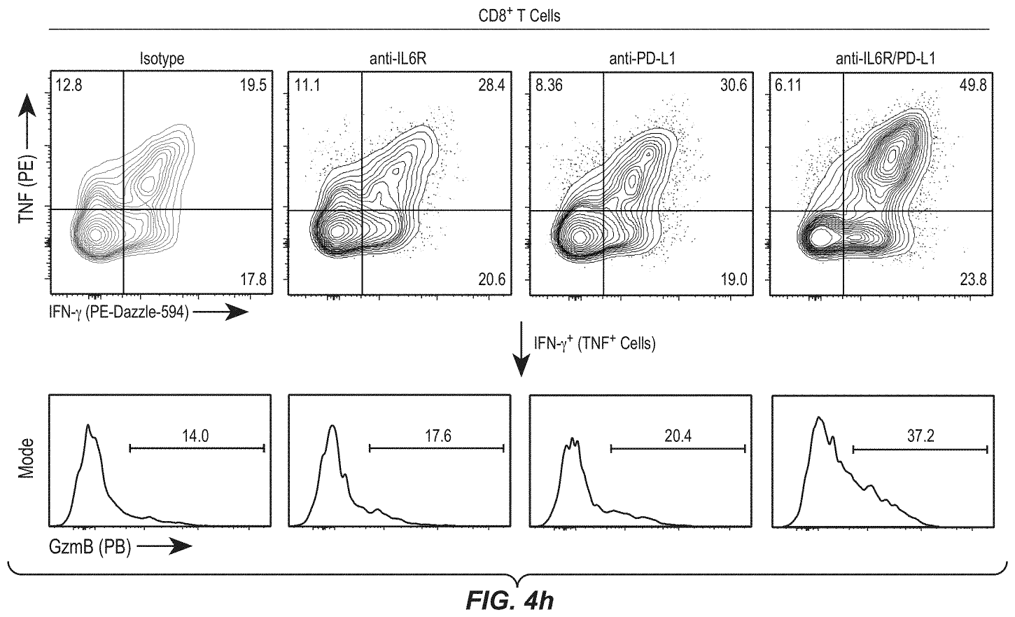

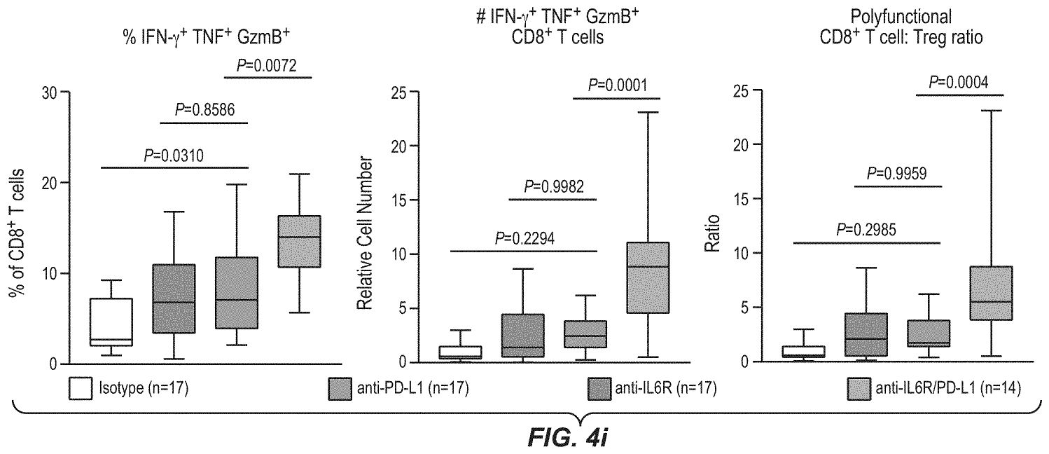

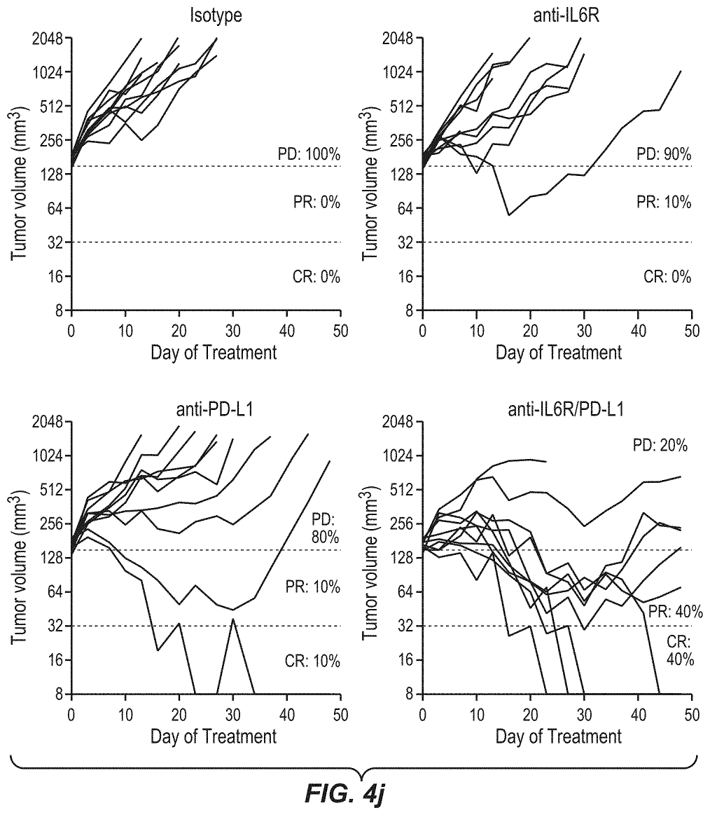

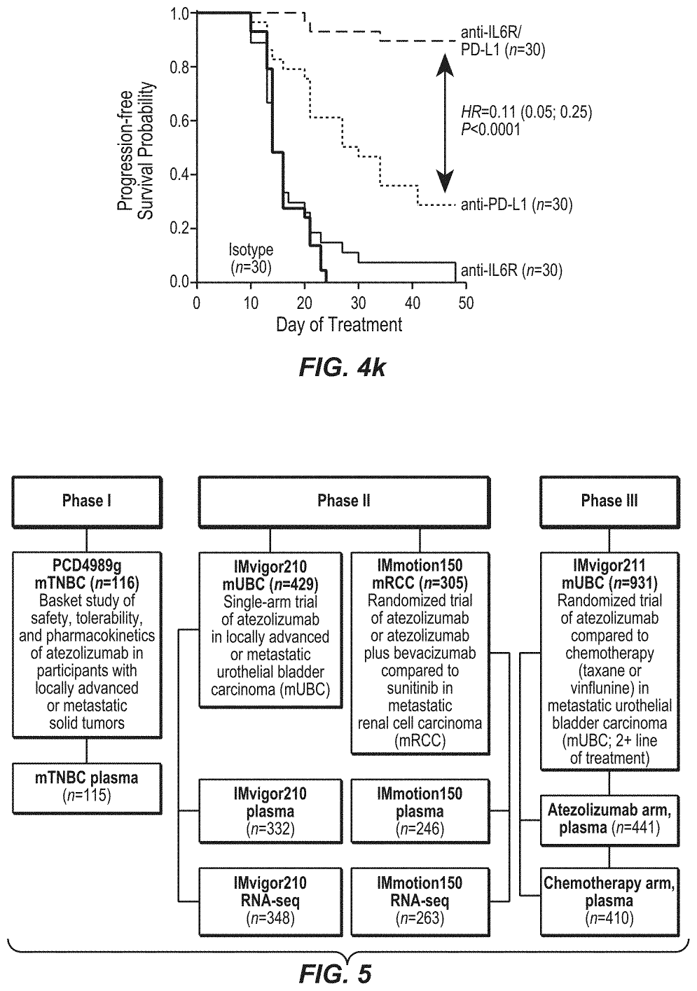

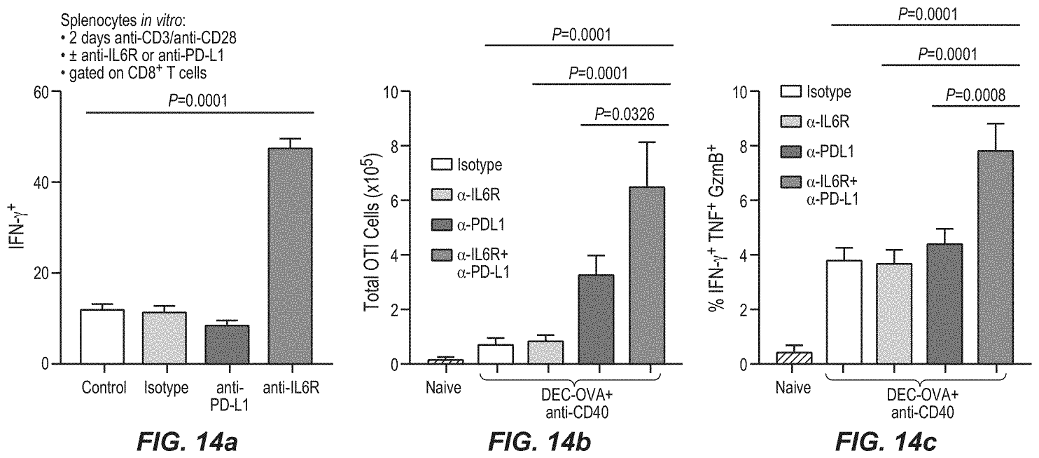

Although PD1/PD-L1 signaling had little effect on CD8⁺ T cell priming in our in vitro culture conditions (FIG. 14a), this axis is known to regulate T cell priming and differentiation in vivo (Goldberg et al. Blood 110:186-192 (2007); Ahn et al. Proc. Natl. Acad. Sci. U.S.A. 115: 4749-4754 (2018). To model the effects of IL-6 and PD-L1 in vivo, we adoptively transferred naive CD8⁺ OT-I T cells (Thy1.1/CD90.1) into wild type (Thy1.2/CD90.2) mice and immunized them with DEC-OVA (ovalbumin conjugated to anti-DEC205 antibody) and agonistic anti-CD40 antibody (FIG. 4a) (Bonifaz et al. J. Exp. Med. 196: 1627-1638 (2002)). Mice were treated with neutralizing antibodies against IL6R and/or PD-L1 one day prior to immunization and again 3 days post-immunization. While neutralization of IL6R alone had little effect, anti-PD-L1 treatment promoted OT-I cell expansion. However, blockade of both IL6R and PD-L1 enhanced expansion and the frequency of polyfunctional effector cells (IFN-γ⁺ TNF⁺ GzmB⁺) to levels that significantly exceeded those observed with anti-PD-L1 alone (FIG. 4b-c and FIGS. 14b-c). These data show that while PD1/PD-L1 signaling constitutes a dominant checkpoint in vivo, IL-6 can act as an additional factor that restricts effector responses when PD-L1 is inhibited.

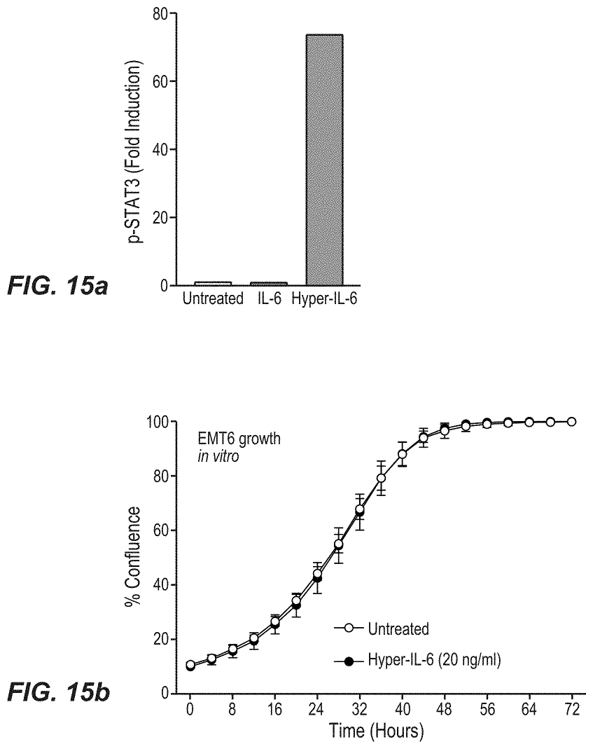

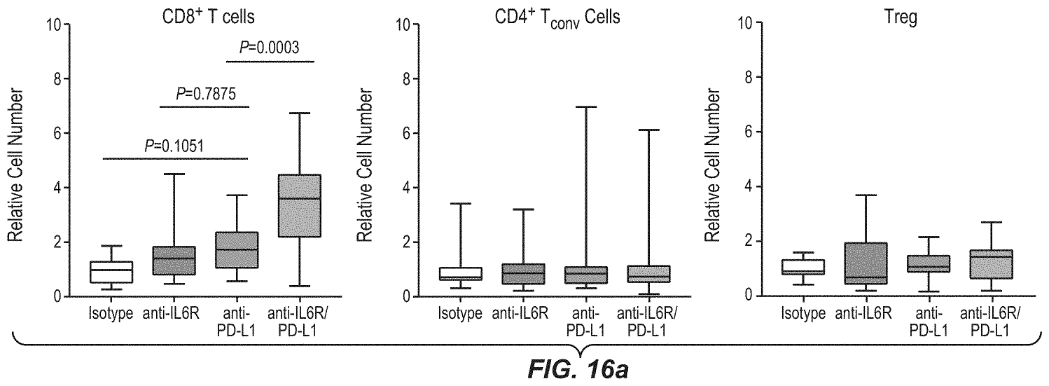

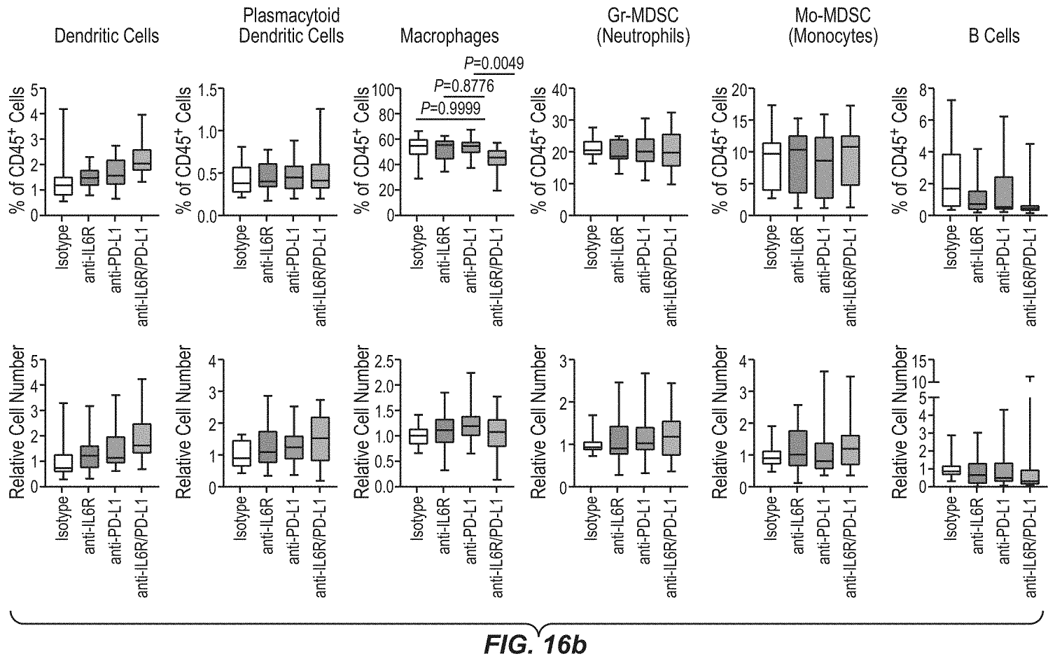

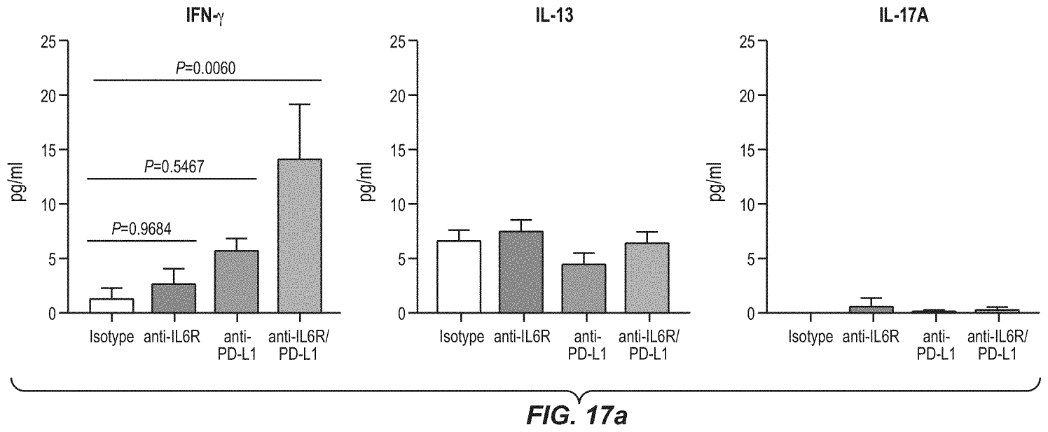

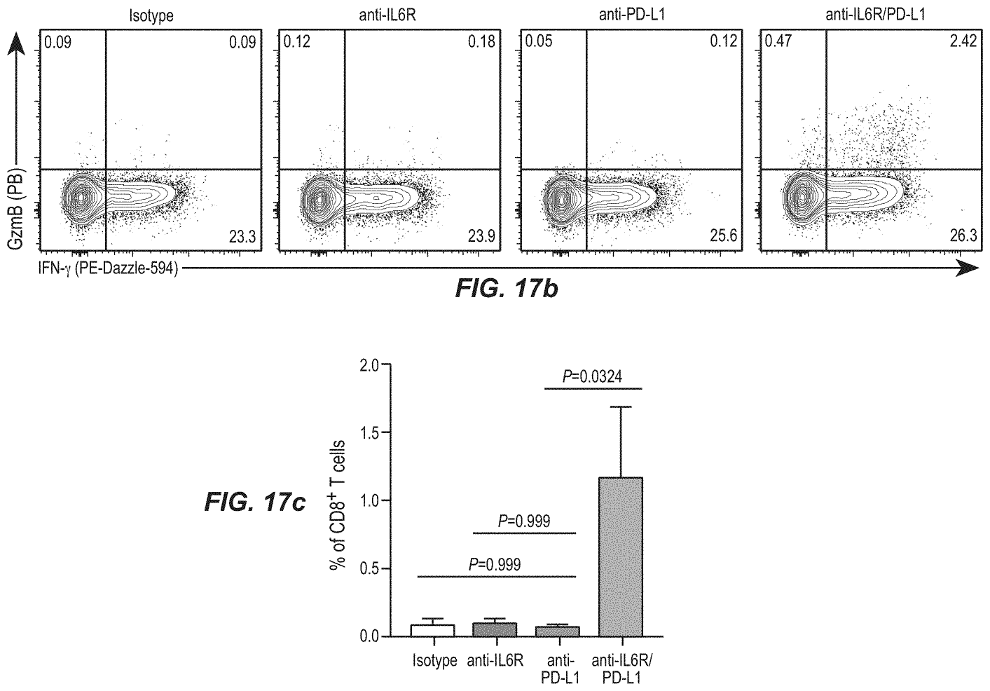

To determine whether IL-6 impairs the effects of anti-PD-L1 therapy in tumor-bearing animals, we examined the syngeneic EMT6 mouse model of triple negative breast cancer (FIG. 4d), which is partially responsive to PD-L1 blockade Mariathasan et al. Nature 554: 544-548 (2018) and, like human cancers, including TNBC, features high levels of plasma IL-6 (FIG. 4e). Importantly, IL-6 had no effect on EMT6 cell proliferation in vitro (FIGS. 15a-b). To characterize the cellular changes driven by anti-IL6R/PD-L1 therapy of established tumors, we profiled tumor-infiltrating leukocytes (TIL) by flow cytometry after 11 days of treatment, at which time tumor volumes remained comparable between treatment groups (FIG. 4d). While single-agent anti-IL6R or anti-PD-L1 treatment had little effect on anti-tumor immune responses, combination therapy drove significant increases in both the frequency and absolute number of tumor-infiltrating CD8⁺ T cells (FIG. 4f-g, FIG. 16a). Furthermore, combination treatment promoted a polyfunctional cytotoxic phenotype (GzmB⁺ IFN-γ⁺ TNF⁺) in CD8⁺ T cells (FIG. 4h-i), consistent with the DEC-OVA immunization model. Notably, with the exception of a slight reduction in macrophage frequency, we did not observe reproducible changes in the frequencies or abundance of other TIL populations during combination therapy, including Foxp3+ CD4+ regulatory T cells (Treg) and conventional CD4⁺ T-helper cells (FIG. 16b). As such, the ratio of polyfunctional CD8⁺ T cells to Treg was increased over 3-fold during combination therapy compared to anti-PD-L1 treatment alone (FIG. 4i). Consistent with these findings, plasma IFN-γ concentration was significantly elevated in mice treated with combined anti-IL6R/anti-PD-L1 therapy, while effector cytokines associated with type 2 (IL-13) and type 17 (IL-17A) immunity were unchanged (FIG. 17a). Likewise, activated CD8⁺ T cell frequencies in tumor-draining lymph nodes were significantly elevated only in mice that received combination therapy (FIGS. 17b-c).

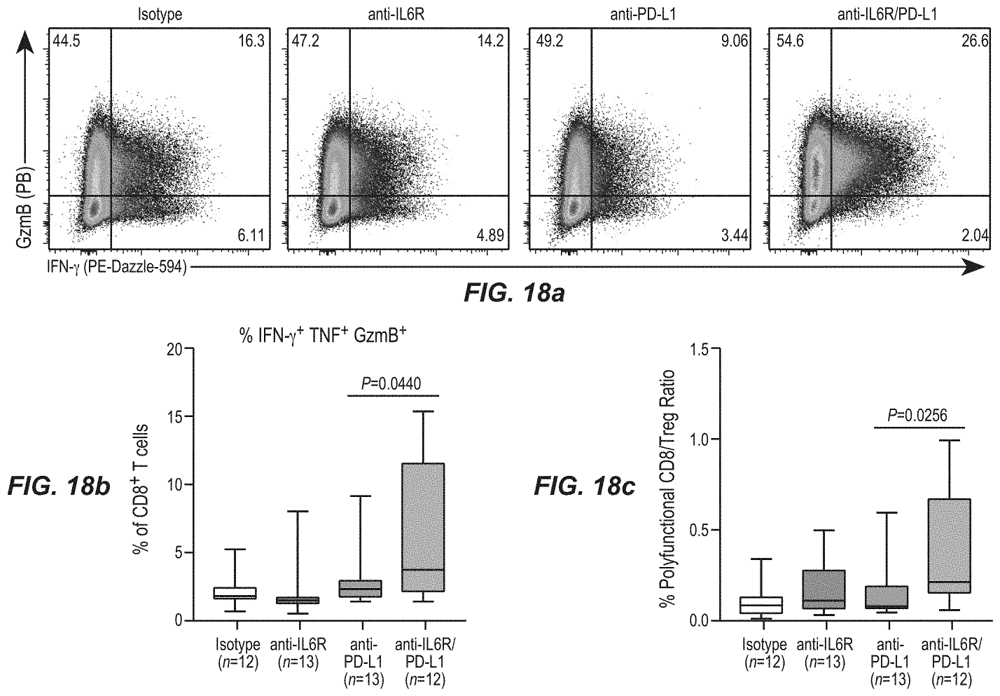

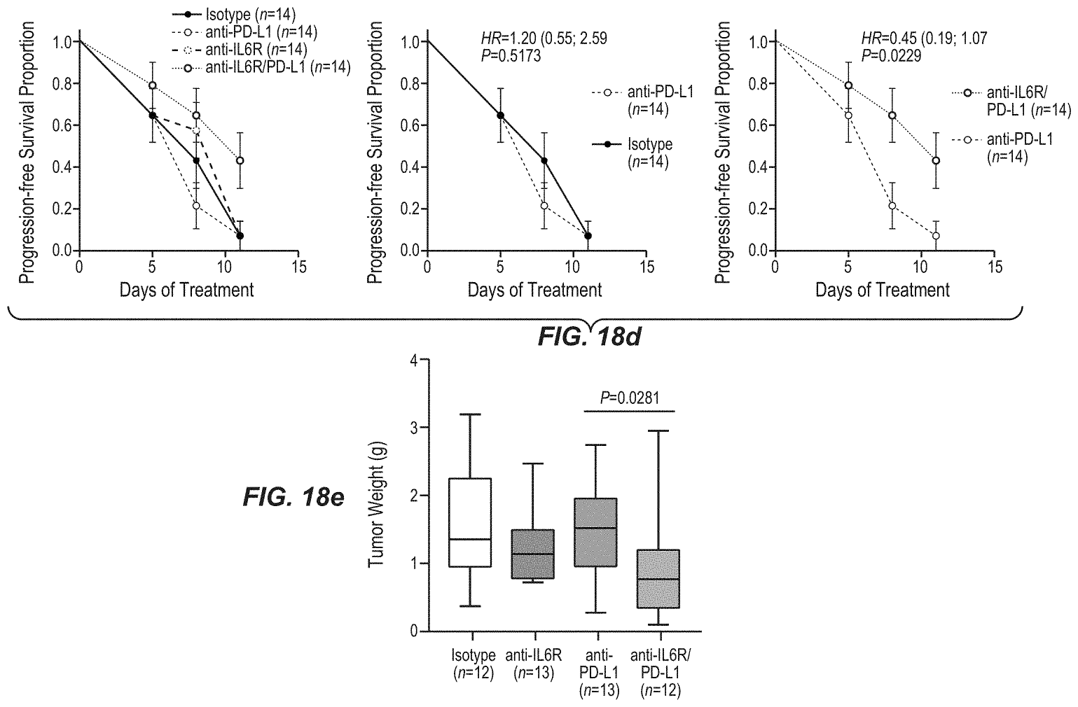

To determine the effect of combination therapy on tumor control, we performed a series of long-term efficacy studies in which mice with large established EMT6 tumors (150-250 mm³ volume) were treated twice weekly with anti-IL6R and/or anti-PD-L1 antibodies for 3 weeks. Tumor burden was tracked after treatment cessation for a further 4 weeks (FIG. 4d). While anti-IL6R had little effect on EMT6 tumor growth, and anti-PD-L1 provided modest anti-tumor efficacy, combined blockade of IL6R and PD-L1 drove robust disease control, with a 3-4-fold increase in the frequency of partial or complete tumor regression and significantly increased progression-free survival compared to anti-PD-L1 alone (HR: 0.11, 95% CI: 0.05, 0.25; P<0.0001) (FIG. 4j-k). We next examined the CT26 colon tumor model, which is highly resistant to anti-PD-L1 therapy. Consistent with the EMT6 model, combined anti-IL6R/anti-PD-L1 therapy of established CT26 tumors (˜150 mm³ volume) promoted high frequencies of polyfunctional tumor-infiltrating CD8⁺ T cells and high CD8-to-Treg ratios, while anti-PD-L1 therapy alone had no effect (FIGS. 18a-c). Likewise, while anti-PD-L1 monotherapy had no therapeutic efficacy, combined anti-IL6R/PD-L1 therapy attenuated CT26 tumor growth (FIGS. 18d-e). Collectively, our data from multiple pre-clinical models show that IL-6 can attenuate CD8⁺ T cell responses and promote resistance to anti-PD-L1 therapy. Combined blockade of IL-6 and PD-L1 enhances CD8⁺ T cell effector function and significantly improves tumor growth inhibition compared to anti-PD-L1 treatment alone.

### Example 6: Discussion of Examples 2 to 5

The data presented here indicate that IL-6 can potentially drive resistance to a PD-1 axis binding antagonist. In this comprehensive evaluation of large clinical studies, it is shows that plasma and intratumoral IL-6 are associated with worse outcome to atezolizumab monotherapy in mTNBC, mUC and mRCC, even in patients whose tumors harboured pre-existing CD8⁺ T cells. This effect was independent of clinical prognostic factors. Moreover, increases in plasma IL-6 concentration during therapy correlated with worse clinical outcome to atezolizumab, but not to chemotherapy. Thus, in addition to established predictive factors such as T cell infiltration, baseline and on-treatment levels of IL-6 and its target gene CRP may be valuable biomarkers of clinical resistance to a PD-1 axis binding antagonist that can be assessed routinely in clinical laboratories.

The mechanisms by which IL-6 impairs anti-PD-L1 efficacy are likely diverse. For example, previous preclinical studies reported that IL-6 inhibits anti-tumor Th1 responses by CD4⁺ T cells (Tsukamonto (2018), supra; Tsukamoto et al. Cancer Res 77: 2279-2291 (2017)). These data from multiple preclinical models indicate that IL-6 can additionally attenuate the effector function of CD8⁺ T cells. Similarly, scRNA-seq analysis of PBMCs from cancer patients indicated reduced CD8⁺ T cell activation in the presence of elevated plasma IL-6. These data contrast with the well-established pro-inflammatory role of IL-6 in diseases characterized by hyperactive Th17 responses, such as rheumatoid arthritis, emphasizing the context-dependent nature of immune regulation by IL-6 (Hunter & Jones Nat Immunol 16: 448-457 (2015); Schaper & Rose-John Cytokine and Growth Factor Reviews 26: 475-487 (2015)).

Although expression of IL-6 by multiple cell types in mRCC tumors was observed, IL-6 produced outside of the tumor bed may also potentially influence CD8⁺ T cell function and anti-tumor responses. Indeed, lymph node fibroblastic reticular cells were recently shown to regulate CD8⁺ T cell metabolism and survival via production of IL-6 (Brown et al. Nat Immunol 20: 1668-1680 (2019)). Moreover, recent analyses of T cell clonality in tumors and peripheral blood have shown that expanded clonotypes found in the tumor are also present in peripheral blood (Wu, T. et al. Peripheral T cell expansion predicts tumor infiltration and clinical response. Nature, In press (2019)), and that in check point inhibitor-treated tumors, T cell expansion in response to therapy may be driven by clones that are newly recruited to the tumor bed (Yost et al. Nature Med 25: 1251-1259 (2019)). Thus, circulating IL6 may also contribute to reduced activation potential of intratumoral T cells recruited from the periphery.

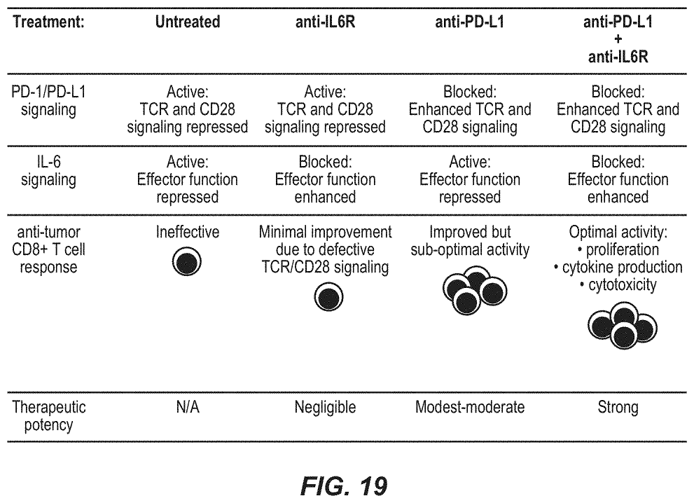

The findings herein show that IL-6 is an additional factor that limits the potency of anti-tumor CD8⁺ T cell responses through selective inhibition of effector function (FIG. 19). Because IL6R blockade only affected CD8⁺ T cell responses in vivo in the context of anti-PD-L1 treatment, the PD-1/PD-L1 axis is likely dominant over IL-6 signaling. Without being bound by any one theory, it is postulated that during blockade of PD-1 or PD-L1, TCR and CD28 signaling is enhanced, but acquisition of potent effector function is restricted by IL-6-driven STAT3 signaling. Combined blockade of PD-1/PD-L1 and IL-6 signaling thus permits both efficient TCR/CD28 signaling and effector polarization, promoting effective anti-tumor responses (FIG. 19). The precise molecular mechanism by which IL-6/STAT3 signaling restricts effector function remains to be defined.

The STAT3-driven inhibitory effect of IL-6 on CD8⁺ T cell effector function makes it an attractive therapeutic target for combination with PD-1 axis binding antagonists, and represents a distinct mechanism of action compared to other factors that restrict PD-1 axix binding antagonist efficacy through indirect means, such as suppression of intratumoral T cell infiltration by TGFβ and VEGF, and recruitment of inhibitory myeloid cells by VEGF, IL-1β, and IL-8. Thus, the combination of IL-6 blockade and PD-1 axis blockade warrants further clinical investigation in cancer patients, with potential for improved therapeutic efficacy in diverse forms of cancer characterized by elevated IL-6 and/or CRP.

### Example 7: Effect of IL-6 Conditioning on CD8⁺ T Cell Effector Function

Bulk splenocytes or spleen-derived CD8⁺ T cells from wild type C57BL/6J mice were cultured in base RPMI 1640 medium with 10% fetal bovine serum (control), or in medium supplemented with 10 ng/ml recombinant mouse IL-6 or 20 ng/ml recombinant mouse hyper-IL-6 (IL-6/IL-6R fusion protein). After 24 hours (the “pretreatment” period), cells were centrifuged, medium was discarded, and cells were cultured with anti-CD3 and anti-CD28 antibodies in the presence of 10 ng/ml human IL-2 for 3 days to activate T cells (the “activation” period). IL-6 or hyper-IL-6 was added to cultures as indicated. Control conditions correspond to cells cultured without IL-6 or hyper-IL-6 for the entire experiment. CD8⁺ T cells were evaluated for cytokine expression by flow cytometry after incubation for 4 hours with brefeldin A.

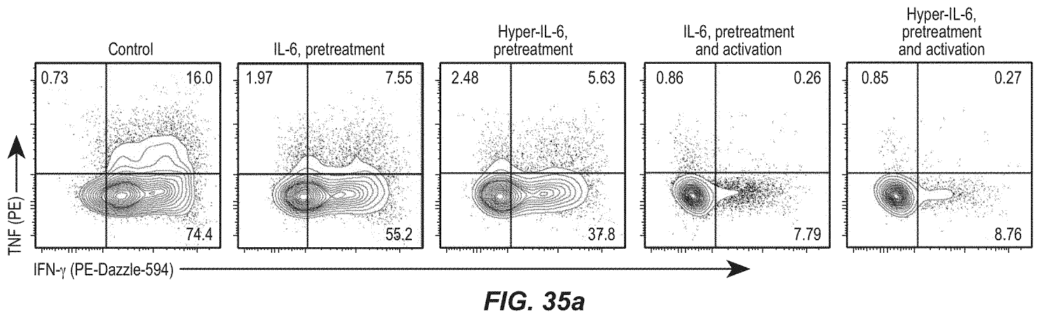

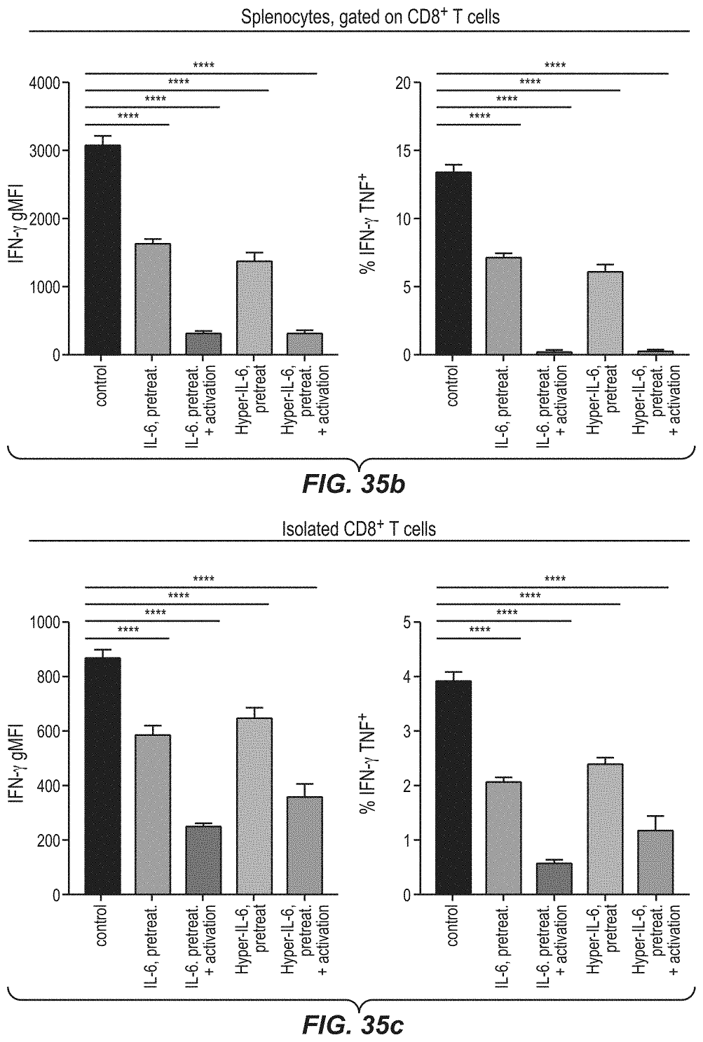

The results are shown in FIGS. 35 a-c. (FIG. 35a) Representative flow cytometry plots of IFN-γ and TNF expression in CD8⁺ T cells from bulk splenocytes. (FIG. 35b) IFN-γ mean fluorescence intensity and frequency of IFN-γ/TNF co-expression in CD8⁺ T cells from bulk splenocytes. (FIG. 35c) IFN-γ mean fluorescence intensity and frequency of IFN-γ/TNF co-expression in CD8⁺ T cells cultured in isolation. In FIGS. 35b and c, bars represent mean+/−s.e.m. from n=8 replicate cultures (control) or n=4 replicate cultures (all other conditions). Groups were compared to control conditions using one-way ANOVA with Dunnett's multiple comparisons test (****0.0001). Data are representative of two independent experiments.

Although the strongest inhibitory effect is seen when IL-6 or hyper-IL6 is present during the anti-CD3/CD28 stimulation period, cells pre-treated with IL-6 also show a blunted effector phenotype, even if IL-6 is withdrawn during anti-CD3/28 stimulation. Based on these data, IL-6 can potentially act on resting T cells as well as on stimulated T cells. This supports administering anti-IL6 receptor therapy prior to anti-PD-L1, to provide sufficient time to relieve IL-6-mediated repression of resting cells prior to treatment with anti-PD-L1 antibodies.

### Example 8. Tocilizumab Combined with Atezolizumab for Urothelial Carcinoma (UC)

This is a Phase Ib/II, open-label, multicenter, randomized, umbrella study in patients with locally advanced or metastatic UC who have progressed during or following a platinum-containing regimen.

Atezolizumab (Atezo) is a humanized immunoglobulin G1 (IgG1) monoclonal antibody that targets programmed death-ligand 1 (PD-L1) and inhibits the interaction between PD-L1 and its receptors, programmed death-1 (PD-1) and B7-1 (also known as CD80), both of which function as inhibitory receptors expressed on T cells. Therapeutic blockade of PD-L1 binding by atezolizumab has been shown to enhance the magnitude and quality of tumor-specific T-cell responses, resulting in improved anti-tumor activity. Atezolizumab has minimal binding to Fc receptors, thus eliminating detectable Fc-effector function and associated antibody-mediated clearance of activated effector T cells. Atezolizumab shows anti-tumor activity in both nonclinical models and cancer patients and is being investigated as a potential therapy in a wide variety of malignancies. Atezolizumab is being studied as a single agent in the advanced cancer and adjuvant therapy settings, as well as in combination with chemotherapy, targeted therapy, and cancer immunotherapy. Atezolizumab has been generally well tolerated. Adverse events with potentially immune-mediated causes consistent with an immunotherapeutic agent, including rash, influenza-like illness, endocrinopathies, hepatitis or transaminitis, pneumonitis, colitis, hypophysitis, myocarditis, and myasthenia gravis, have been observed. To date, these events have been manageable with treatment.

Tocilizumab (TCZ) is a recombinant humanized, anti-human monoclonal antibody of the IgG1 subclass directed against the soluble and membrane-bound interleukin 6 receptor (IL-6R). Tocilizumab binds specifically to both soluble IL-6R (sIL-6R) and membrane-bound IL-6R (mIL-6R) and has been shown to inhibit sIL-6R and mIL-6R-mediated signaling. Interleukin 6 (IL-6) is a pleiotropic pro-inflammatory, multifunctional, cytokine produced by a variety of cell types. It has been shown to be involved in such diverse physiological processes as T-cell activation; induction of acute phase proteins; stimulation of hematopoietic precursor cell growth and differentiation; proliferation of hepatic, dermal and neural cells; bone metabolism; lipid metabolism; hepatoprotection; and fibrosis. Elevated tissue and serum levels of IL-6 have been implicated in the disease pathology of several inflammatory and autoimmune disorders, including rheumatoid arthritis, Castleman's disease, systemic juvenile idiopathic arthritis, polyarticular juvenile idiopathic arthritis, giant cell arteritis, Takayasu arteritis, systemic sclerosis, and cytokine release syndrome.

Atezolizumab is administered at a fixed dose of 840 mg every 2 weeks (Q2W) (840 mg on Days 1 and 15 of each 28-day cycle). The average concentration following the 840 mg Q2W dosage is expected to be equivalent to that of 1200 mg every 3 weeks (Q3W), the approved dosage for atezolizumab. The atezolizumab drug product will be supplied by the Sponsor as a sterile liquid in a single-use, 20-mL glass vial. The vial contains approximately 20 mL (1200 mg) of atezolizumab solution. Atezolizumab injection for intravenous use is a sterile, preservative-free, colorless to slightly yellow solution in single-dose vials. Each 20 mL vial contains 1200 mg of atezolizumab and is formulated in glacial acetic acid (16.5 mg), L-histidine (62 mg), polysorbate 20 (8 mg), and sucrose (821.6 mg), with a pH of 5.8.

Tocilizumab will be administered by iv infusion at a dose of 8 mg/kg every 4 weeks (Q4W) on Day 1 of each 28-day cycle, the approved dose for tocilizumab for the treatment of RA. Tocilizumab will be supplied by the Sponsor as a sterile solution at a concentration of 20 mg/mL in single-use vials containing 4.0, 10.0, or 20.0 mL. Tocilizumab injection is a sterile, clear, colorless to pale yellow, preservative-free solution for further dilution prior to intravenous infusion with a pH of approximately 6.5. Each single-dose vial, formulated with a disodium phosphate dodecahydrate/sodium dihydrogen phosphate dihydrate buffered solution, is available at a concentration of 20 mg/mL containing 80 mg/4 mL, 200 mg/10 mL, or 400 mg/20 mL of Tocilizumab. Each mL of solution contains polysorbate 80 (0.5 mg), sucrose (50 mg), and Water for Injection, USP.

Patients in the Atezo+TCZ arm will receive treatment as outlined in the following table until unacceptable toxicity or loss of clinical benefit. Tocilizumab will be administered first. Atezolizumab will be administered 2 hours after the conclusion of the tocilizumab infusion.

### Treatment Regimen for Atezo+TCZ Arm

| | Dose, Route, and Regimen |
|---|---|
| Cycle Length | (drugs listed in order of administration) |
| 28 days | Tocilizumab 8 mg/kg IV on Day 1 |
| | Atezolizumab 840 mg IV on Days 1 and 15 |

Atezo + TCZ = atezolizumab plus tocilizumab.

Tocilizumab will be administered by IV infusion at a dose of 8 mg/kg Q4W on Day 1 of each 28-day cycle. Each patient will receive 8 mg/kg tocilizumab (or 4 mg/kg in certain circumstances), with a maximum dose of 800 mg tocilizumab (for patients weighing >100 kg). The last recorded body weight of a patient should be used for calculating tocilizumab volumes for each infusion. The dose administered should be within 10% of the calculated dose. No premedication is required before tocilizumab infusions.

Atezolizumab will be administered by IV infusion at a fixed dose of 840 mg on Days 1 and 15 of each 28-day cycle. Atezolizumab should be administered 2 hours after the completion of the tocilizumab infusion.

Inclusion Criteria
Histologically documented, locally advanced (T4b, any N; or any T, N2-N3) or metastatic UC (M1, Stage IV) (also termed TCC or urothelial cell carcinoma of the urinary tract; including renal pelvis, ureters, urinary bladder, and urethra)
   Patients with mixed histologies are required to have a dominant transitional cell pattern.
   Locally advanced bladder cancer must be inoperable on the basis of involvement of pelvic sidewall or adjacent viscera (clinical Stage T4b) or bulky nodal metastasis (N2-N3).
Disease progression during or following treatment with no more than one platinum-containing regimen (e.g., GC, MVAC, CarboGem) for inoperable, locally advanced or metastatic UC or disease recurrence
   A regimen is defined as patients receiving at least two cycles of a platinum-containing regimen.
   Patients who received prior adjuvant/neoadjuvant chemotherapy and progressed within 12 months of treatment with a platinum-containing adjuvant/neoadjuvant regimen will be considered as second-line patients.

| Primary Efficacy Objective | Corresponding Endpoint |
|---|---|
| To evaluate the efficacy of immunotherapy- | Objective response rate, defined as the |
| based treatment combinations | proportion of patients with a CR or PR on two |
| | consecutive occasions □4 weeks apart, as |
| | determined according to RECIST v1.1 |
| Secondary Efficacy Objective | Corresponding Endpoints |
| To evaluate the efficacy of immunotherapy- | PFS after randomization, a defined as |
| based treatment combinations | the time from randomization to the first |
| | occurrence of disease progression or death |
| | from any cause (whichever occurs first), as |
| | determined by the investigator according to |
| | RECIST v1.1 |
| | OS after randomization, a defined as |
| | the time from randomization to death from |
| | any cause |
| | OS rate at specific timepoints (e.g., 12 |
| | months), defined as the proportion of |
| | patients who have not experienced death |
| | from any cause at that timepoint |
| | DOR, defined as the time from the first |
| | occurrence of a documented objective |
| | response to disease progression or death |
| | from any cause (whichever occurs first), as |
| | determined according to RECIST v1.1 |
| | Disease control, defined as stable disease ≥18 |
| | weeks or a CR or PR, as determined |
| | according to RECIST v1.1 |
| Safety Objective | Corresponding Endpoints |
| To evaluate the safety of immunotherapy- | Incidence, nature, and severity of adverse |
| based treatment combinations | events and laboratory abnormalities, with |
| | severity determined according to NCI |
| | CTCAE v4.0 |
| | Change from baseline in vital signs |
| | Change from baseline in targeted clinical |
| | laboratory test results |

ADA = anti-drug antibody; CR = complete response; DOR = duration of response; iRECIST = immune; NCI CTCAE v4.0 = National Cancer Institute Common Terminology Criteria for Adverse Events, Version 4.0; OS = overall survival; PFS = progression-free survival; PK = pharmacokinetic; PR = partial response; RECIST v1.1 = Response Evaluation Criteria in Solid Tumors, Version 1.1.
^{a} For the mandatory serial-biopsy arms, PFS and OS will be determined from the time of treatment initiation (rather than time of randomization).

Patients may have received no more than two prior regimens of treatment (including the required platinum-based regimen) for their advanced or metastatic UC. Patients must have demonstrated disease progression during or following all prior regimen(s).

Patients with disease progression following chemoradiotherapy must demonstrate progression outside the prior radiotherapy port.

Fifteen patients have been randomly assigned to a control arm (atezolizumab [Atezo]) or Atezo in combination with tocilizumab (TCZ). The objectives and corresponding endpoints of the study are summarized in the table below. It is expected that the combined treatment with Atezo and TCZ disclosed herein will achieve one or more of the efficacy endpoints: overall response rate (CR and/or PR) and/or improved PFS and/or OS compared with Atezo alone (i.e. treatment without TCZ), while having acceptable toxicity.

### Example 9: Tocilizumab Combined with Atezolizumab for Metastatic Triple Negative Breast Cancer (mTNBC)

This study will evaluate the efficacy, safety, and pharmacokinetics of Atezolizumab in combination with Tocilizumab in patients with metastatic triple-negative breast cancer (TNBC). Nab-Paclitaxel chemotherapy will also be administered. Patients are PD-L1 positive. Patients will receive treatment as outlined in the table below. The Atezo and TCZ formulations are as described in Example 8.

### Treatment Regimen for Atezo+Nab-Pac+TCZ

| | Dose, Route, and Regimen |
|---|---|
| Cycle Length | (drugs listed in order of administration) |
| 28 Days | Tocilizumab 8 mg/kg by IV infusion on Day 1 |
| | Atezolizumab 840 mg by IV infusion on Days 1 |
| | and 15a |
| | Nab-paclitaxel 100 mg/m2 by IV infusion on Days |
| | 1, 8, and 15b |
| | Atezo + Nab-Pac + TCZ = atezolizumab plus |
| | nab-paclitaxel (nanoparticle albuminbound |
| | paclitaxel) plus tocilizumab. |

aAtezolizumab should be administered 2 hours after completion of the tocilizumab infusion on Day 1 of the first two cycles. On Day 1 of subsequent cycles, atezolizumab can be administered after completion of the tocilizumab infusion.
bNab-paclitaxel will be administered after completion of the atezolizumab infusion.

### Inclusion Criteria

ECOG Performance Status of 0 or 1
Metastatic or inoperable locally advanced, histologically documented TNBC (absence of HER2, estrogen receptor [ER], and progesterone receptor [PR] expression), as defined by the American Society for Clinical Oncology/College of American Pathologists guidelines HER2 negativity is defined as either of the following by local laboratory assessment: In situ hybridization non-amplified (ratio of HER2 to CEP17<2.0 or single-probe average HER2 gene copy number <4 signals/cell) or Immunohistochemistry (IHC) 0 or IHC 1+; and ER and PR negativity are defined as <1% of cells expressing hormonal receptors via IHC analysis
For patients in the 1L PD-L1+ cohort: no prior systemic treatment for metastatic or inoperable locally advanced TNBC
For patients in the 1L PD-L1+ cohort: positive PD-L1 expression, defined as >1% of the tumor area occupied by PD-L1+ expressing tumor-infiltrating immune cells of any intensity, as determined through use of the U.S. Food and Drug Administration approved or CE-marked Ventana PD-L1 (SP142) Assay.
   For management of drug-related toxicities, the dose of nab-paclitaxel may be reduced by 25 mg/m² (one dose level) up to two times and the dose of tocilizumab may be reduced by 4 mg/m² (one dose level) up to one time, as outlined in the following table.

### Suggested Dose Reductions for Nab-Paclitaxel and Tocilizumab

| | | |
|---|---|---|
| | First Dose | Second Dose |
| Initial Dose | Reduction | Reduction |

| | | | | | |
|---|---|---|---|---|---|
| Nab-paclitaxel | 100 | mg/m² | 75 | mg/m² | 50 mg/m² |
| Tocilizumab | 8 | mg/kg | 4 | mg/kg | NA |

NA = not applicable; nab-paclitaxel = nanoparticle albumin-bound paclitaxel.

The objectives and endpoints are described in the following table. It is expected that the patient treated with the combination herein will achieve one or more of the efficacy endpoints: overall response rate (CR and/or PR) and/or improved PFS and/or OS compared with Atezo+NabPac (i.e. without TCZ), while having acceptable toxicity

### Objectives and Corresponding Endpoints

| Primary Efficacy Objective | Corresponding Endpoint |
|---|---|
| To evaluate the efficacy of Atezo and | ORR, defined as the proportion of patients with a |
| TCZ (plus chemotherapy) | complete response or partial response, as determined |
| | by the investigator according to RECIST v1.1 |
| Secondary Efficacy Objective | Corresponding Endpoints |
| To evaluate the efficacy of Atezo and | PFS, defined as the time from randomization to the |
| TCZ (plus chemotherapy) | date of the first recorded occurrence of disease |
| | progression or death from any cause (whichever |
| | occurs first), as determined by the investigator |
| | according to RECIST v1.1 |
| | DCR, defined as proportion of patients with stable |
| | disease for ≥18 weeks or a confirmed complete or |
| | partial response, as determined by the investigator |
| | according to RECIST v1.1 |
| | OS, defined as the time from randomization to death |
| | from any cause |
| | OS at specific timepoints (e.g., 12 months) |
| | DOR, defined as the time from the first occurrence of |
| | a documented objective response to the first recorded |
| | occurrence of disease progression or death from any |
| | cause (whichever occurs first), as determined by the |
| | investigator according to RECIST v1.1 |
| Safety Objective | Corresponding Endpoints |
| To evaluate the safety of Atezo and | Incidence, nature, and severity of adverse events and |
| TCZ (plus chemotherapy) | laboratory abnormalities, with severity determined |
| | according to NCI CTCAE v4.0 |
| | Change from baseline in vital signs and ECG |
| | parameters |
| | Change from baseline in targeted clinical laboratory |
| | test results |

ADA = anti-drug antibody; DCR = disease control rate; DOR = duration of response; iRECIST = modified RECIST v1.1 for immune-based therapeutics; ORR = objective response rate; OS = overall survival; PFS = progression-free survival; NCI CTCAE v4.0 = National Cancer Institute Common Terminology Criteria for Adverse Events, Version 4.0; PK = pharmacokinetic; RECIST = Response Evaluation Criteria in Solid Tumors.

### Example 10: Atezolizumab Combined with Tocilizumab and Bevacizumab for Advanced Liver Cancer (Morpheus Liver)

Liver cancer is the fifth most common cancer and the second most frequent cause of cancer-related death globally, with 854,000 new cases and 810,000 deaths per year. Hepatocellular carcinoma (HCC) is the most prevalent form of primary liver cancer and represents approximately 90% of all primary hepatic malignancies. Less prevalent primary liver cancers include intrahepatic cholangiocarcinoma (iCCA), angiosarcoma, and hepatoblastoma.

Study Y040245 (IMbrave150) is an ongoing, randomized Phase III study evaluating atezolizumab plus bevacizumab versus sorafenib as first-line treatment in patients with advanced or metastatic HCC. This study is the first to demonstrate a statistically significant and clinically meaningful improvement in OS and progression-free survival (PFS) for a novel treatment combination in a head-to-head comparison with sorafenib. At the time of the primary analysis, the risk of death was reduced by 42% for the atezolizumab plus bevacizumab arm compared with the sorafenib arm (stratified hazard ratio [HR]=0.58 [95% CI: 0.42 to 0.79]; p=0.0006; median OS, not estimable [NE] vs. 13.24 months). Independent-Review Facility-assessed PFS per RECIST v1.1 also demonstrated a statistically significant and clinically meaningful improvement favoring the combination treatment (stratified HR=0.59 [95% CI: 0.47 to 0.76]; p<0.0001; median PFS, 6.83 vs. 4.27 months). Overall, the atezolizumab plus bevacizumab combination in HCC was generally well tolerated with manageable toxicities and the safety profile was consistent with the known risks of the individual study treatments and with the underlying disease (Cheng et al. IMbrave150: Efficacy and Safety Results From a Ph 3 Study Evaluating Atezolizumab (atezo)+Bevacizumab (bev) vs Sorafenib (Sor) as First Treatment (tx) for Patients (pts) With Unresectable Hepatocellular Carcinoma (HCC). Proceedings of ESMO Asia 2019: 22-24 Nov. 2019 [cited: 27 Nov. 2019]; Singapore. Available from: https://www.esmo.org/Oncology-News/Atezolizumab-in-Combination-with-Bevacizumab-Provides-Superior-Outcome-Compared-with-Sorafenib-in-Unresectable-HCC).

This is a Phase Ib/II, open-label, multicenter, randomized umbrella study in patients with locally advanced or metastatic hepatocellular carcinoma (HCC) who have not received prior systemic therapy for their disease.

Patients will be randomly assigned to a control arm (atezolizumab plus bevacizumab [Atezo+Bev]) or treatment arm consisting of atezolizumab and bevacizumab in combination with tiragolumab (Atezo+Bev+Tira) or tocilizumab (Atezo+Bev+TCZ).

Control Arm (Atezo+Bev)

Patients in the atezolizumab plus bevacizumab (Atezo+Bev) arm will receive treatment as outlined in the following table.

### Treatment Regimen for Atezo+Bev Arm

| | Dose, Route, and Regimen |
|---|---|
| Cycle Length | (drugs listed in order of administration) |
| 21 days | Atezolizumab 1200 mg by IV infusion on Day 1 |
| | Bevacizumab 15 mg/kg by IV infusion on Day 1 |

Atezo + Bev = atezolizumab plus bevacizumab.

Atezo+Bev+TCZ

Patients in the atezolizumab plus bevacizumab plus tocilizumab (Atezo+Bev+TCZ) arm will receive treatment as outlined in the following table:

### Treatment Regimen for Atezo+Bev+TCZ Arm

| | Dose, Route, and Regimen |
|---|---|
| Cycle Length | (drugs listed in order of administration) |
| 21 Days | Atezolizumab 1200 mg by IV infusion on Day 1 |
| | Bevacizumab 15 mg/kg by IV infusion on Day 1 |
| | Tocilizumab 8 mg/kg by IV infusion on Day 1 ^{a} |

^{a} On Day 1 of Cycle 1, tocilizumab will be administered 60 minutes after completion of the bevacizumab infusion. The interval between subsequent infusions will be 30 minutes if the previous bevacizumab infusion was given without premedication and tolerated without an IRR or 60 minutes if the patient experienced an IRR with the previous bevacizumab infusion.

### Inclusion Criteria

Patients must meet all of the following criteria:
Age ≥18 years.
ECOG Performance Status of 0 or 1 within 7 days prior to treatment.
Locally advanced or metastatic and/or unresectable HCC with diagnosis confirmed by histology/cytology or clinically by AASLD criteria in cirrhotic patients
   For cirrhotic patients with no histological confirmation of diagnosis, clinical confirmation is required per AASLD criteria.
Child-Pugh class A within 7 days prior to randomization
Disease that is not amenable to curative surgical and/or locoregional therapies
   Patients with progressive disease after surgical and/or locoregional therapies are eligible.
No prior systemic treatment (including systemic investigational agents) for HCC
   Prior treatment with herbal therapies, including traditional Chinese medicines, with anti-cancer activity noted in the label are allowed, provided that these medications are discontinued prior to randomization.
Life expectancy ≥3 months.

The efficacy and safety objectives and endpoints are described in the following tables. It is expected that the patient treated with the triple combination of Atezo+Bev+Toci herein will achieve one or more of the efficacy endpoints (ORR, PFS, OS, DOR and/or disease control) compared with placebo Atezo+Bev (i.e. without TCZ), while having acceptable toxicity.

### Objectives and Corresponding Endpoints

| Primary Efficacy Objective | Corresponding Endpoint |
|---|---|
| To evaluate the efficacy of immunotherapy- | ORR, defined as the proportion of patients |
| based treatment combinations | with a complete response or partial response on |
| | two consecutive occasions 4 weeks apart, as |
| | determined by the investigator according to |
| | RECIST v1.1 |
| Secondary Efficacy Objective | Corresponding Endpoints |
| To evaluate the efficacy of immunotherapy- | PFS after randomization, defined as the |
| based treatment combinations | time from randomization to the first occurrence of |
| | disease progression or death from any cause |
| | (whichever occurs first), as determined by the |
| | investigator according to RECIST v1.1 |
| | OS after randomization, defined as the |
| | time from randomization to death from any cause |
| | OS at specific timepoints (e.g., 6 months) |
| | DOR, defined as the time from the first |
| | occurrence of a documented objective response to |
| | disease progression or death from any cause |
| | (whichever occurs first), as determined by the |
| | investigator according to RECIST v1.1 |
| | Disease control, defined as stable disease |
| | for 12 weeks or a complete or partial response, as |
| | determined by the investigator according to |
| | RECIST |
| Exploratory Efficacy Objective | Corresponding Endpoints |
| To evaluate the efficacy of immunotherapy- | ORR, PFS, DOR, and disease control as |
| based treatment combinations | determined by the investigator according to |
| | iRECIST and HCC mRECIST |
| Safety Objective | Corresponding Endpoints |
| To evaluate the safety of | Incidence, nature, and severity of adverse events and |
| immunotherapy-based treatment | laboratory abnormalities, with severity determined |
| combinations | according to NCI CTCAE v5.0 |
| | Change from baseline in vital signs and ECG |
| | parameters |
| | Change from baseline in targeted clinical laboratory |
| | test results |
| Exploratory | |
| Pharmacokinetic Objectives | Corresponding Endpoints |
| To characterize the PK profile of | Plasma or serum concentration of each drug (as |
| drugs that are administered as part of | appropriate) at specified timepoints |
| an immunotherapy-based treatment | |
| combination | |
| To evaluate potential relationships | Relationship between plasma or serum concentration |
| between drug exposure and the | or PK parameters for each drug (as appropriate on |
| efficacy and safety of | the basis of available data) and efficacy endpoints |
| immunotherapy-based treatment | Relationship between plasma or serum concentration |
| combinations | or PK parameters for each drug (as appropriate on |
| | the basis of available data) and safety endpoints |
| Exploratory | |
| Immunogenicity Objectives | Corresponding Endpoint |
| To evaluate the immune response to | For drugs for which ADA formation is measured: |
| drugs that are administered as part of | presence of ADAs during the study relative to the |
| an immunotherapy-based treatment | presence of ADAs at baseline |
| combination | |
| To evaluate potential effects of ADAs | For drugs for which ADA formation is measured: |
| | relationship between ADA status and efficacy, safety, |
| | or PK endpoints |

ADA = anti-drug antibody; DOR = duration of response; HCC = hepatocellular carcinoma; HCC mRECIST = HCC-specific modified RECIST; iRECIST = modified RECIST v1.1 for immune-based therapeutics; NCI CTCAE v5.0 = National Cancer Institute Common Terminology Criteria for Adverse Events, Version 5.0; ORR = objective response rate; OS = overall survival; PFS = progression-free survival; PK = pharmacokinetic; RECIST = Response Evaluation Criteria in Solid Tumors.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of treating a cancer patient comprising administering to the patient a combination of an IL-6 antagonist and a PD-1 axis binding antagonist in an amount effective to treat the cancer.

2. The method of claim 1, wherein the cancer is breast cancer.

3. The method of claim 1, wherein the cancer is a triple negative breast cancer.

4. The method of claim 1, wherein the cancer is bladder cancer.

5. The method of claim 1, wherein the cancer is urothelial carcinoma.

6. The method of claim 1, wherein the cancer is kidney cancer.

7. The method of claim 1, wherein the cancer is renal cell carcinoma.

8. The method of claim 1, wherein the patient has C-reactive protein (CRP) level above the upper limit of normal.

9. The method of claim 8, wherein the patient has ≥3 mg/L CRP.

10. The method of claim 9, wherein the patient has ≥10 mg/L CRP.

11. The method of claim 8, wherein CRP is measured by enzyme-linked immunosorbent assay (ELISA) assay of a blood sample from the patient.

12. The method of claim 1, wherein the patient has IL-6 level above the upper limit of normal.

13. The method of claim 12, wherein the patient has ≥10 μg/mL IL-6.

14. The method of claim 13, wherein the patient has ≥15 μg/mL IL-6.

15. The method of claim 12, wherein IL-6 is measured by enzyme-linked immunosorbent assay (ELISA) assay of a blood sample from the patient.

16. The method of any claim 1, wherein the patient expresses PD-L1.

17. The method of claim 16, wherein the patient has PD-L1 stained tumor cells (TC) or tumor-infiltrating immune cells (IC).

18. The method of claim 17, wherein the patient has PD-L1 stained IC covering 1% of the tumor area.

19. The method of claim 17, wherein the patient has PD-L1 stained IC covering 5% of the tumor area.

20. The method of claim 1, wherein the IL-6 antagonist is administered to the patient prior to the administration of the PD-1 axis binding antagonist.

21. The method of claim 1, wherein the patient does not have cytokine release syndrome (CRS).

22. The method of claim 1, wherein the IL-6 antagonist is an anti-IL6 receptor antibody.

23. The method of claim 22, wherein the anti-IL6 receptor antibody is tocilizumab, satralizumab, sarilumab, NI-1201, or vobarilizumab.

24. The method of claim 22, wherein the anti-IL6 receptor antibody is tocilizumab.

25. The method of claim 1, wherein the IL-6 antagonist binds IL-6.

26. The method of claim 25, wherein the IL-6 binding antagonist is siltuximab, sirukumab, olokizumab, clazakizumab, EBI-031, or olamkicept.

27. The method of claim 1, wherein the PD-L1 axis binding antagonist is a PD-L1 binding antagonist, a PD-1 binding antagonist, or a PD-L2 binding antagonist.

28. The method of claim 27, wherein the PD-L1 axis binding antagonist is a PD-L1 binding antagonist.

29. The method of claim 28, wherein the PD-L1 binding antagonist inhibits the binding of PD-L1 to both PD-1 and B7-1.

30. The method of claim 28, wherein the PD-L1 binding antagonist is an antibody.

31. The method of claim 30, wherein the PD-L1 antibody is atezolizumab, MDX-1105, MEDI4736 (durvalumab), or MSB0010718C (avelumab).

32. The method claim 1, wherein the PD-L1 axis binding antagonist is a PD-1 binding antagonist.

33. The method of claim 32, wherein the PD-1 binding antagonist is MDX-1106 (nivolumab), MK-3475 (pembrolizumab), MEDI-0680 (AMP-514), PDR001, REGN2810, BGB-108, or AMP-224.

34. The method of claim 1, wherein the IL-6 antagonist is an IL-6 receptor binding antibody and the PD-1 axis binding inhibitor is a PD-L1 binding antibody.

35. The method of claim 34, wherein the IL-6 receptor binding antibody is tocilizumab and the PD-L1 binding antibody is atezolizumab.

36. The method of claim 35, wherein the tocilizumab is administered by intravenous (iv) infusion at a dose of 8 mg/kg every 4 weeks (Q4w) on Day 1 of each 28-day cycle.

37. The method of claim 36, wherein tocilizumab is administered until disease progression or unacceptable toxicity.

38. The method of claim 35, wherein the atezolizumab is administered intravenously (iv) at a fixed dose of 840 mg every 2 weeks (Q2W) on Days 1 and 15 of each 28-day cycle.

39. The method of claim 38, wherein atezolizumab is administered until disease progression or unacceptable toxicity.

40. The method of claim 35, wherein the tocilizumab is administered first and atezolizumab is administered after the tocilizumab administration.

41. The method of claim 40, wherein the atezolizumab is administered about two hours after the conclusion of the tocilizumab administration.

42. The method of claim 1, wherein the treatment achieves an objective response rate (ORR).

43. The method of claim 42, wherein the treatment achieves a complete response (CR).

44. The method of claim 42, wherein the treatment achieves a partial response (PR).

45. The method of claim 1, wherein the treatment extends progression free survival (PFS) or overall survival (OS).

46. The method of claim 45, wherein PFS or OS is extended to a greater extent than treatment without the IL-6 antagonist.

47. The method of claim 1, wherein treatment results in an increased abundance of CD8⁺ T cells in the patient relative to that of a subject who has not been administered the IL-6 antagonist.

48. A method of treating a cancer patient comprising administering to the patient a combination of an anti-IL6 receptor antibody and an anti-PD-L1 antibody in an amount effective to treat the cancer.

49. The method of claim 48, wherein the cancer is breast cancer, urothelial carcinoma, or renal cell carcinoma.

50. The method of claim 48, wherein the anti-IL6 receptor antibody is tocilizumab and the anti-PD-L1 antibody is atezolizumab.

51. A method of treating a cancer patient with C-reactive protein (CRP) level above the upper limit of normal comprising administering to the patient a combination of an anti-IL6 receptor antibody and an anti-PD-L1 antibody in an amount effective to treat the cancer.

52. A method of treating advanced urothelial carcinoma in a cancer patient comprising administering to the patient a combination of tocilizumab and atezolizumab in an amount effective to treat the cancer.

53. A method of treating triple negative breast cancer (TNBC) in a cancer patient comprising administering to the patient a combination of tocilizumab, atezolizumab, and chemotherapy in an amount effective to treat the cancer.

54. The method of claim 53, wherein the chemotherapy comprises a taxane.

55. The method of claim 54, wherein the taxane is nanoparticle albumin-bound paclitaxel (nab paclitaxel).

56. A method of reducing or preventing therapeutic resistance to a PD-1 axis binding antagonist in a cancer patient comprising administering the PD-1 axis binding antagonist to the patient in combination with an IL-6 antagonist in an amount effective to treat the cancer.

57. A method of treating cancer in a cancer patient comprising administering to the patient a combination of atezolizumab, bevacizumab, and tocilizumab in an amount effective to treat the cancer.

58. The method of claim 57, wherein cancer is liver cancer.

59. The method of claim 58, wherein the liver cancer is hepatocellular carcinoma (HCC).


SEQUENCE LISTING (34 sequences)

SEQ ID NO: 1 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, Artificial Sequence [sequence omitted]

SEQ ID NO: 4 - protein, 108 aa, Artificial Sequence
  source: /note=“Description of Artificial Sequence Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 5 - protein, 10 aa, Artificial Sequence
  source: /note=“Description of Artificial Sequence Synthetic peptide”
  VARIANT (6)..(6): /replace=“Gly”
  SITE (1)..(10): /note=“Variant residues given in the sequence have no preference with respect to those in the annotations for variant positions”
  source: /note=“See specification as filed for detailed description of substitutions and preferred embodiments”
GFTFSDSWIH

SEQ ID NO: 6 - protein, 18 aa, Artificial Sequence
  source: /note=“Description of Artificial Sequence Synthetic peptide”
  VARIANT (4)..(4): /replace=“Leu”
  VARIANT (10)..(10): /replace=“Ser”
  SITE (1)..(18): /note=“Variant residues given in the sequence have no preference with respect to those in the annotations for variant positions”
  source: /note=“See specification as filed for detailed description of substitutions and preferred embodiments”
AWISPYGGSTYYADSVKG

SEQ ID NO: 7 - protein, 9 aa, Artificial Sequence
  source: /note=“Description of Artificial Sequence Synthetic peptide”
RHWPGGFDY

SEQ ID NO: 8 - protein, 25 aa, Artificial Sequence [sequence omitted]

SEQ ID NO: 9 - protein, 13 aa, Artificial Sequence
  source: /note=“Description of Artificial Sequence Synthetic peptide”
WVRQAPGKGLEWV

SEQ ID NO: 10 - protein, 32 aa, Artificial Sequence
  source: /note=“Description of Artificial Sequence Synthetic polypeptide”
RFTISADTSKNTAYLQMNSLRAEDTAVYYCAR

SEQ ID NO: 11 - protein, 11 aa, Artificial Sequence
  source: /note=“Description of Artificial Sequence Synthetic peptide”
WGQGTLVTVSS

SEQ ID NO: 12 - protein, 11 aa, Artificial Sequence
  source: /note=“Description of Artificial Sequence Synthetic peptide”
  VARIANT (5)..(5): /replace=“Val”
  VARIANT (6)..(6): /replace=“Ile”
  VARIANT (7)..(7): /replace=“Asn”
  VARIANT (9)..(9): /replace=“Phe”
  VARIANT (10)..(10): /replace=“Leu”
  SITE (1)..(11): /note=“Variant residues given in the sequence have no preference with respect to those in the annotations for variant positions”
  source: /note=“See specification as filed for detailed description of substitutions and preferred embodiments”
RASQDVSTAVA

SEQ ID NO: 13 - protein, 7 aa, Artificial Sequence
  source: /note=“Description of Artificial Sequence Synthetic peptide”
  VARIANT (4)..(4): /replace=“Thr”
  VARIANT (6)..(6): /replace=“Ala”
  SITE (1)..(7): /note=“Variant residues given in the sequence have no preference with respect to those in the annotations for variant positions”
  source: /note=“See specification as filed for detailed description of substitutions and preferred embodiments”
SASFLYS

SEQ ID NO: 14 - protein, 9 aa, Artificial Sequence
  source: /note=“Description of Artificial Sequence Synthetic peptide”
  VARIANT (3)..(3): /replace=“Gly” or “Phe” or “Ser”
  VARIANT (4)..(4): /replace=“Tyr” or “Phe” or “Trp”
  VARIANT (5)..(5): /replace=“Asn” or “Ala” or “Thr” or “Gly” or “Phe” or “Ile”
  VARIANT (6)..(6): /replace=“Val” or “Pro” or “Thr” or “Ile”
  VARIANT (8)..(8): /replace=“Trp” or “Arg” or “Pro” or “Thr”
  SITE (1)..(9): /note=“Variant residues given in the sequence have no preference with respect to those in the annotations for variant positions”
  source: /note=“See specification as filed for detailed description of substitutions and preferred embodiments”
QQYLYHPAT

SEQ ID NO: 15 - protein, 23 aa, Artificial Sequence
  source: /note=“Description of Artificial Sequence Synthetic peptide”
DIQMTQSPSSLSASVGDRVTITC

SEQ ID NO: 16 - protein, 15 aa, Artificial Sequence
  source: /note=“Description of Artificial Sequence Synthetic peptide”
WYQQKPGKAPKLLIY

SEQ ID NO: 17 - protein, 32 aa, Artificial Sequence
  source: /note=“Description of Artificial Sequence Synthetic polypeptide”
GVPSRFSGSGSGTDFTLTISSLQPEDFATYYC

SEQ ID NO: 18 - protein, 11 aa, Artificial Sequence
  source: /note=“Description of Artificial Sequence Synthetic peptide”
FGQGTKVEIKR

SEQ ID NO: 19 - protein, 10 aa, Artificial Sequence
  source: /note=“Description of Artificial Sequence Synthetic peptide”
GFTFSDSWIH

SEQ ID NO: 20 - protein, 18 aa, Artificial Sequence
  source: /note=“Description of Artificial Sequence Synthetic peptide”
AWISPYGGSTYYADSVKG

SEQ ID NO: 21 - protein, 9 aa, Artificial Sequence
  source: /note=“Description of Artificial Sequence Synthetic peptide”
RHWPGGFDY

SEQ ID NO: 22 - protein, 11 aa, Artificial Sequence
  source: /note=“Description of Artificial Sequence Synthetic peptide”
RASQDVSTAVA

SEQ ID NO: 23 - protein, 7 aa, Artificial Sequence
  source: /note=“Description of Artificial Sequence Synthetic peptide”
SASFLYS

SEQ ID NO: 24 - protein, 9 aa, Artificial Sequence
  source: /note=“Description of Artificial Sequence Synthetic peptide”
QQYLYHPAT

SEQ ID NO: 25 - protein, 122 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 26 - protein, 10 aa, Artificial Sequence
  source: /note=“Description of Artificial Sequence Synthetic peptide”
FGQGTKVEIK

SEQ ID NO: 27 - protein, 30 aa, Artificial Sequence
  source: /note=“Description of Artificial Sequence Synthetic polypeptide”
EVQLVESGGGLVQPGGSLRLSCAASGFTFS

SEQ ID NO: 28 - protein, 14 aa, Artificial Sequence
  source: /note=“Description of Artificial Sequence Synthetic peptide”
WVRQAPGKGLEWVA

SEQ ID NO: 29 - protein, 15 aa, Artificial Sequence
  source: /note=“Description of Artificial Sequence Synthetic peptide”
WGQGTLVTVSSASTK

SEQ ID NO: 30 - protein, 447 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, Artificial Sequence
  source: /note=“Description of Artificial Sequence Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, Artificial Sequence [sequence omitted]

SEQ ID NO: 33 - protein, 448 aa, Artificial Sequence
  source: /note=“Description of Artificial Sequence Synthetic polypeptide”
  MOD_RES (1)..(1): Pyroglutamic Acid
XVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 8 aa, Gallus gallus
SIINFEKL